(19) United States
(12) Patent Application Publication
(10) Pub. No.: US 2023/0348415 A1
(43) Pub. Date: Nov. 2, 2023

(54) BISPECIFIC ANTAGONISTS OF RETINOL-BINDING PROTEIN 4 THAT STABILIZE TRANSTHYRETIN TETRAMERS, THEIR PREPARATION, AND USE IN THE TREATMENT OF COMMON AGE-RELATED COMORBIDITIES

(71) Applicants: The Trustees of Columbia University in the City of New York, New York, NY (US); Albany College of Pharmacy and Health Sciences in the City of Albany, Albany, NY (US)

(72) Inventors: Konstantin Petrukhin, New Windsor, NY (US); Boglarka Racz, Brooklyn, NY (US); Andras Varadi, Brooklyn, NY (US); Parthasarathy Muthuraman, Albany, NY (US); Arun Raja, Albany, NY (US); Christopher L. Cioffi, Albany, NY (US)

(73) Assignees: The Trustees of Columbia University in the City of New York, New York, NY (US); Albany College of Pharmacy and Health Sciences in the City of Albany, Albany, NY (US)

(21) Appl. No.: 18/017,006

(22) PCT Filed: Jul. 20, 2021
(86) PCT No.: PCT/US2021/042300

§ 371 Date: Jan. 19, 2023

Related U.S. Application Data
(60) Provisional application No. 63/054,218, filed on Jul. 20, 2020.

(51) Int. Cl. C07D 401/04; C07D 403/04; C07D 487/10; C07D 401/14; C07D 403/14; A61P 27/02

(52) U.S. Cl. CPC C07D 401/04; C07D 403/04; C07D 487/10; C07D 401/14; C07D 403/14; A61P 27/02

(57) ABSTRACT

The present invention provides a compound having the structure:

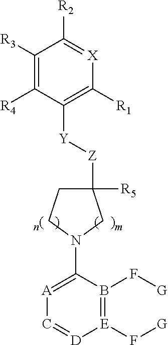

wherein
   X is CR₆ or N;
   R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈ or —NHC(S) R₈,
wherein R₇ is, H, -(alkyl), —OH, —O(alkyl), —NH₂, —NH(alkyl) or —N(alkyl)₂, and
wherein R₈ is, -(alkyl), —O-(alkyl), —NH₂, —NH(alkyl) or —N(alkyl)₂;
   Y is O, S, N, NH, or a bond;
   Z is O, S, N, NH, (CH₂)ₒ, or a bond;
   R₅ is H, OH, halogen, alkyl, or R₅ is (CH₂)ₚ and is bound to Y when Y is N to form a ring together with Z;
   o and p are independently 0, 1, 2, or 3;
   m and n are independently 0, 1, 2, 3, or 4;
   A, C, and D are each independently N or CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, —CO(O-alkyl);
B and E are N, CR₉, or CFG wherein at least one of B or E is CFG;
   F is absent or present, and when present is

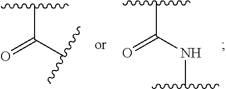

G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein each R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl), —N(alkyl)₂,
or a pharmaceutically acceptable salt thereof.

This application claims priority of U.S. Provisional Application No. 63/054,218, filed Jul. 20, 2020, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Age-related macular degeneration (AMD) is the most common cause of blindness in developed countries. Age-dependent accumulation of cytotoxic lipofuscin bisretinoids in the retina may significantly contribute to pathogenesis of the atrophic form of AMD. The essential vitamin all-trans-retinol (vitamin A, 1) (FIG. 1) serves as a precursor for the biosynthesis of retinoic acid (2) (Steinmetz, A. C. et al. 2001; Clagett-Dame, M. & DeLuca, H. F. 2002; Wolf, G. 1984), 11-cis-retinal (3) (Kiser, P. D. et al. 2014; Tsin, A. et al. 2018), and many other key retinoids involved in multiple cellular processes and numerous critical biological functions throughout the body. Bisretinoid synthesis in the eye depends on the influx of all-trans-retinol (1) from the serum to the retina. Formation of the tertiary retinol-binding protein 4 (RBP4)-transthyretin (TTR)-retinol complex in the serum is required for this influx. Reducing circulating levels of RBP4 and 1 via selective antagonists could modulate the visual cycle, reduce the rate of cytotoxic bisretinoid formation in the retinal pigment epithelium (RPE), and halt geographic atrophy in patients with atrophic (dry) age-related macular degeneration (AMD) and Stargardt disease (Radu, R. A. et al. 2005; Palczewski, K. 2010; Petrukhin, K. 2013; Petrukhin, K. 2007). Competitive RBP4 antagonists that displace 1 prevent the formation of the holo-RBP4-TTR complex, thereby inducing reductions in circulating RBP4 and 1 levels via rapid renal clearance. A diminished influx of 1 to the RPE results in a reduction of cytotoxic bisretinoid accumulation in the retina, which is believed to underlie parts of the pathophysiology of dry AMD and Stargardt disease (Radu, R. A. et al. 2005; Dobri, N. et al. 2013; Racz, B. et al. 2018; Young, R. W. 1987; Dorey, C. K. et al. 1989; Holz, F. G. et al. 2001; Holz, F. G. et al. 1999; Holz, F. G. et al. 2007; Schmitz-Valckenberg, S. et al. 2009; Finnemann, S. C. et al. 2002; Suter, M. et al. 2000; Sparrow, J. R. et al. 2003; Sparrow, J. R. et al. 2012; Delori, F. C. 1995; Weng, J. et al. 1999; Sparrow, J. R. & Cai, B. 2001; Bergmann, M. et al. 2004; Sparrow, J. R. et al. 1999; De, S. & Sakmar, T. P. 2002; Vives-Bauza, C. et al. 2008; Zhou, J. et al. 2006; Radu, R. A. et al. 2011; Ben-Shabat, S. et al. 2002; Rozanowska, M. et al. 1995; Sparrow, J. R. et al. 2002; Dontsov, A. E. et al. 2009). The approach is supported by proof-of-concept data obtained for fenretinide (5) (FIG. 2), which was studied in the preclinical Abca4^{-/-} transgenic mouse model of enhanced retinal lipofuscinogenesis (Radu, R. A. et al. 2005) and in an extended Phase II clinical trial with dry AMD patients (Berni, R. & Formelli, F. 1992; Adams, W. R. et al. 1995; Mata, N. L. et al. 2013). The non-retinoid RBP4 antagonist A1120 (6) (Motani, A. et al. 2009) was found to lower circulating RBP4 plasma levels in rodents by >70% and reduce retinal bisretinoid accumulation in Abca4^{-/-} mice (Dobri, N. et al. 2013). Selective and orally bioavailable non-retinoid RBP4 antagonists 7 (Cioffi, C. L. et al. 2014) and BPN-14136 (8) (Cioffi, C. L. et al. 2015) displayed favorable pharmacokinetic (PK) profiles and induced dose-dependent reductions in rodent plasma RBP4 levels in both acute and chronic dosing studies. Compound 8 also robustly lowered serum RBP4 levels and exhibited excellent pharmacokinetic-pharmacodynamic (PK/PD) correlations in non-human primates upon oral administration (Racz, B. et al. 2020). Compound 8 inhibited lipofuscin bisretinoid synthesis with concomitant normalization of retinal complement system protein expression in the Abca4^{-/-} mouse model without altering visual cycle kinetics at doses inducing maximal serum RBP4 reduction (Racz, B. et al. 2018).

In addition to transporting 1 to targeted tissues, RBP4 has also been identified as an adipokine and epidemiological evidence suggests that moderately elevated levels of the protein positively correlate with type 2 diabetes (Graham, T. E. et al. 2006; Yang, Q. et al. 2005), obesity (Aeberli, I. et al. 2007), insulin resistance (Kowalska, I. et al. 2008), cardiovascular disease (Ingelsson, E. et al. 2009; Qi, Q. et al. 2007; Norseen, J. et al. 2012), and hepatic steatosis (Lee, S. A. et al. 2016). Thus, the pharmacological reduction of circulating RBP4 serum levels may also hold promise for the treatment of a myriad of metabolic diseases. It has recently been reported that RBP4 antagonist 10 significantly lowered serum RBP4 levels in rodents (>80%), reduced the concentration of circulating RBP4 produced in the adipose tissue, and demonstrated efficacy in the transgenic adi-hRBP4 murine model of hepatic steatosis, suggesting that it may have therapeutic utility for the treatment of non-alcoholic fatty liver disease (NAFLD) (Cioffi, C. L. et al. 2019).

The deposition of amyloid aggregates derived from either mutant (TTRm) or wild-type (TTRwt) underlies TTR amyloidosis (ATTR) diseases such as senile systemic amyloidosis (SSA), peripheral polyneuropathy (ATTR-PN), and cardiomyopathy (ATTR-CM) (Johnson, S. M. et al. 2005; Foss, T. R. et al. 2005; Falk, R. H. et al. 1997; Brunjes, D. L. et al. 2016; Ton, V. K. et al. 2014). The breakage of the dimer-dimer interface in TTR tetramers constitutes the first step in the TTR tetramer dissociation process that leads to TTR misfolding. Approximately 50% of serum TTR is associated with holo-RBP4 and the formation of the tertiary holo-RBP4-TTR complex is suggested to stabilize this fraction of serum TTR tetramers protecting them from dissociation and misfolding (White, J. T. & Kelly, J. W. 2001; Hyung, S. J. et al. 2010). Based on the in vitro observation that RBP4-TTR interaction is capable of conferring an additional stabilization to tetrameric TTR (White, J. T. & Kelly, J. W. 2001; Hyung, S. J. et al. 2010), it seems plausible that the release of TTR tetramers from RBP4-TTR-retinol complexes induced by selective RBP4 antagonists may lead to tetramer destabilization and its enhanced dissociation to dimer subunits. The resulting dimers may then further dissociate into monomers that can misfold, aggregate, oligomerize, and eventually form insoluble TTR amyloid fibrils (White, J. T. & Kelly, J. W. 2001; Hyung, S. J. et al. 2010). While selective RBP4 antagonists can be a safe and effective therapy for the majority of dry AMD patients, this class of compounds may potentially be counter-indicated for a fraction of AMD patients who may be prone to developing ATTR. In addition to individuals with rare genetic forms of transthyretin amyloidosis caused by proamyloidogenic TTR mutations, the use of selective RBP4 antagonists may not be optimal in patients with senile systemic amyloidosis (SSA), a late-onset non-genetic disease associated with misfolding and aggregation of wild-type TTR. SSA affects approximately 25% of patients over the age of 80 (Ruberg, F. L. & Berk, J. L. 2012; Connors, L. H. et al. 2011; Westermark, P. et al. 2003) and based on the high population frequency of this disease and dry AMD, significant comorbidity between the two conditions is expected. In addition, the use of selective RBP4 antagonists may not be optimal in older African-American patients with dry AMD who have the increased chance of carrying a relatively high frequency pro-amyloidogenic V122I mutation in the TTR gene (Buxbaum, J. N. et al. 2017; Alexander, K. M. & Falk, R. H. 2016). It is undesirable for an effective chronic treatment for one of the two conditions to be counter-indicated for the use in patients with another one, and developing an optimal therapy for dry AMD that can be safely used in patients with ATTR comorbidities is an important objective.

The initial and rate-limiting step in ATTR pathophysiology is the sequential dissociation of TTR tetramers (Johnson, S. M. et al. 2005; Foss, T. R. et al. 2005). While thyroxine (4) (FIG. 1) binding was reported to stabilize TTR tetramers (Sekijima, Y. et al. 2003), the majority of TTR in circulation (up to 90%), including TTR in a complex with holo-RBP4, is not bound to its natural ligand (White, J. T. & Kelly, 2001). Current therapeutic approaches to treat ATTR-CM include small molecule kinetic stabilizers of TTR tetramers that bind at the T4 binding sites and increase the energy barrier of tetramer dissociation (Kerschen, P. & Plante-Bordeneuve, V. 2016; Almeida, M. R. et al. 2005; Almeida, M. R. et al. 2004; Johnson, S. M. et al. 2005; Nencetti, S. & Orlandini, E. 2012; Adams, D. et al. 2016). Two orally bioavailable kinetic stabilizers clinically investigated to date include FDA-approved tafamidis (11) (FIG. 3) (Coelho, T. et al. 2016; Nencetti, S. et al. 2013; Lamb, Y. N. & Deeks, E. D. 2019; Bulawa, C. E. et al. 2012), and AG10 (12) (Penchala, S. C. et al. 2013; Miller, M. et al. 2018). TTR stabilizer 11 is currently approved to treat familial amyloid polyneuropathy (FAP) and ATTR-CM patients, and 12 has demonstrated near-complete stabilization of TTR in ATTR-CM patients with symptomatic, chronic heart failure in Phase III clinical trials (Judge, D. P. et al. 2019). In addition, the repurposed FDA-approved non-steroidal anti-inflammatory drug (NSAID) diflunisal (13) (Berk, J. L. et al. 2012) and catechol-O-methyl transferase (COMT) inhibitor tolcapone (14) (Sant'Anna, R. et al. 2016) are examples of additional small molecules that also exhibit TTR tetramer kinetic stabilization activity and have been investigated for clinical efficacy against ATTR-PN.

This invention describes a novel class of non-retinoid bispecific compounds capable of exhibiting dual retinol-binding protein 4 (RBP4) antagonist and transthyretin (TTR) tetramer kinetic stabilization activity for the treatment of dry age-related macular degeneration (AMD) and TTR amyloidosis (ATTR) comorbidities. We here show below that these compounds may provide therapeutic benefits associated with reducing circulating RBP4 levels while simultaneously stabilizing unliganded TTR tetramers released from the holo-RBP4-TTR complex, thus circumventing potential risks of amyloid fibril formation as schematized in FIG. 4. Furthermore, a polypharmacological approach consisting of single bispecific molecule capable of exhibiting dual activity for both targets may present advantages over the co-administration of single agent for each target. Such advantages include improving patient compliance, minimizing complex PK, and avoiding potential drug-drug interactions that could arise from multiple drug intake (Rodrigues, D. A. 2008).

### SUMMARY OF THE INVENTION

The present invention provides a compound having the structure:

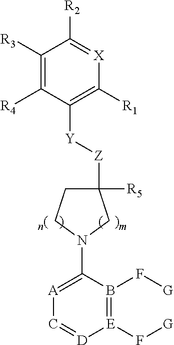

wherein
   X is CR₆ or N;
   R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈ or —NHC(S) R₈,
wherein R₇ is, H, -(alkyl), —OH, —O(alkyl), —NH₂, —NH(alkyl) or —N(alkyl)₂, and
wherein R₈ is, -(alkyl), —O-(alkyl), —NH₂, —NH(alkyl) or —N(alkyl)₂;
   Y is O, S, N, NH, or a bond;
   Z is O, S, N, NH, (CH₂)ₒ, or a bond;
   R₅ is H, OH, halogen, alkyl, or R₅ is (CH₂)ₚ and is bound to Y when Y is N to form a ring together with Z;
   o and p are independently 0, 1, 2, or 3;
   m and n are independently 0, 1, 2, 3, or 4;
   A, C, and D are each independently N or CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, —CO(O-alkyl);
B and E are N, CR₉, or CFG wherein at least one of B or E is CFG;
   F is absent or present, and when present is

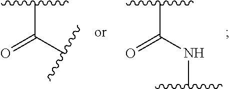

G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein each R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl), —N(alkyl)₂,
   or a pharmaceutically acceptable salt thereof.

### BRIEF DESCRIPTION OF THE FIGURES

FIG. 1. All-trans-retinol (vitamin A) (1), retinoic acid (2) (a vital retinoid involved in morphogenesis), 11-cis-retinal (3) (a key retinoid required for phototransduction) and the thyroid hormone thyroxine (T4) (4).

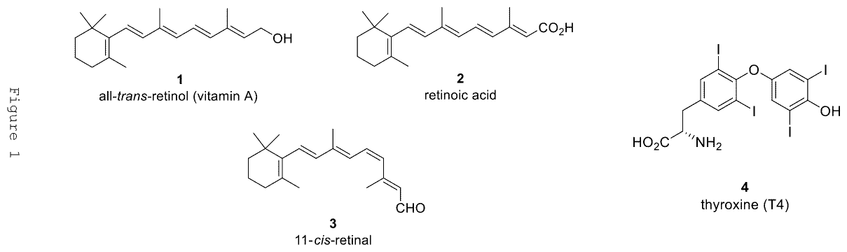

FIG. 2. Examples of previously reported RBP4 antagonists that include fenretinide (5), A1120 (6), non-retinoid [3.3.0]-octahydrocyclopenta[c]pyrrolo RBP4 antagonist 7, BPN-14136 (8), [3.3.0]-octahydrocyclopenta[c]pyrrolo RBP4 antagonist 9, and [1,2,4]triazolo[4,3-a]pyridine 10.

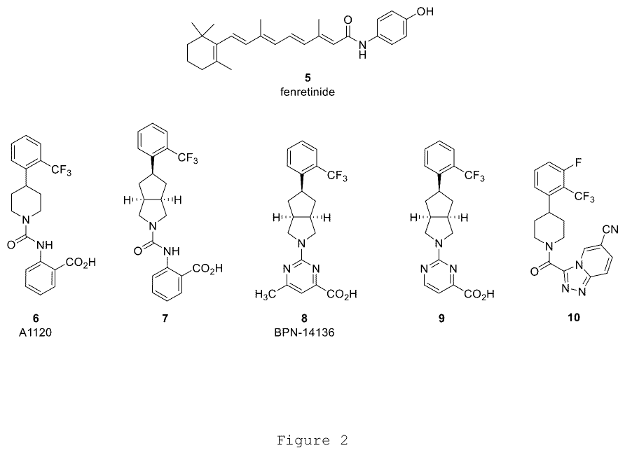

FIG. 3. Examples of reported small molecule TTR tetramer kinetic stabilizers that include tafamidis (11), AG10 (12), diflunisal (13), and tolcapone (14).

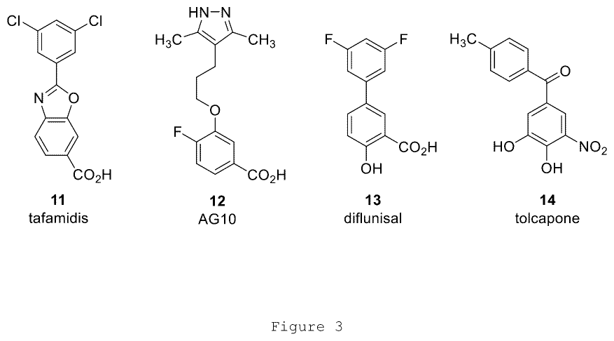

FIG. 4. Bispecific RBP4 antagonists and TTR tetramer kinetic stabilizers for treating RBP4 indications with potential ATTR comorbidities. (A) Schematic illustrating the use of selective RBP4 antagonists for disrupting the holo-RBP4-TTR protein-protein interaction and inducing serum reduction of 1 and RBP4. The concomitant release of unliganded TTR may induce its aggregation, potentially contributing to ATTR in predisposed patients. (B) Bispecific ligands with dual RBP4 antagonist and TTR tetramer kinetic stabilization activity may induce reductions in circulating levels of RBP4 and 1 while also preventing potential TTR aggregation and insoluble amyloid fibril formation.

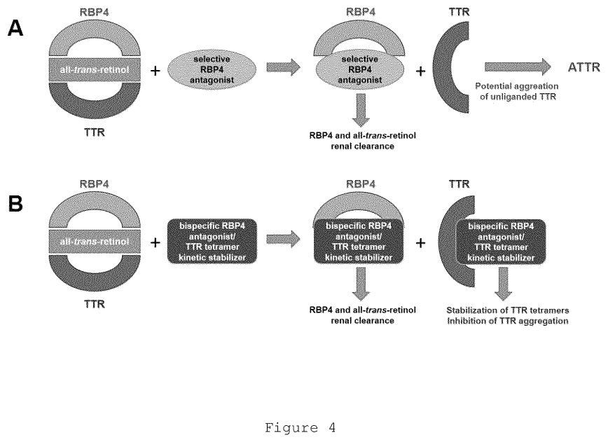

FIG. 5. Pharmacokinetic and pharmacodynamic properties of (±)-44 in mice. (A) Serum RBP4 levels following a single 25 mg/kg oral administration of (±)-44. (B) Blood compound levels following administration of a single oral 5 mg/kg dose of (±)-44. Data represented as the mean±SD. Three mice per treatment group were used in the study.

FIG. 6. Analogue (±)-44 reduces the formation of high molecular weight TTR forms in the acid-induced aggregation assay. (A), TTR protein (5 μg) was aggregated by using acetate buffer (pH 4.0) and incubated for 72 h at 37° C. After incubation in the presence of 50 μM Tafamidis, 50 μM Benzbromarone and 50 μM (±)-44, samples were crosslinked and subjected to SDS-PAGE followed by western blotting with TTR antibodies. The representative blot of at least three independent experiments is presented. (B, C), Bar graphs represent pixel volumes of TTR high molecular weight aggregates (B) and monomers (C). The vertical axes represent the ratio of pixel volume means±S.D. of the scanned bands on the immunoblots in arbitrary units. Statistical significance was determined by one-way ANOVA with Holm-Sidak post hoc test; *, p≤0.05; **, p≤0.01; ***, p≤0.001; ****, p≤0.0001 compare to TTR aggregation (pH 4.0)+DMSO group, #, p≤0.05; ##, p≤0.01; ###, p≤0.001; ####, p≤0.0001 compare to TTR without aggregation (pH 7.5) group.

FIG. 7. Effect of the long-term oral (±)-44 administration in Abca4^{-/-} mice on serum RBP4 levels. Serum RBP4 levels were measured in vehicle-treated Abca4^{-/-} mice (squares) and (±)-44-treated Abca4-mice (triangles) at the indicated time points. Compared with the baseline, statistically significant 79% RBP4 reduction at week 12 was seen in the (±)-44 treatment group (two-way ANOVA with Sidak post hoc test, p<0.0001). Error bars show S.D. Each data point on the graph represents a serum RBP4 concentration from an individual animal.

FIG. 8. Effect of the long-term oral (±)-44 administration on the level of the lipofuscin fluorophore A2E in eyes of the Abca4^{-/-} mice. Bisretinoids were extracted from the eyecups of vehicle-treated wild type mice (circles), vehicle-treated Abca4^{-/-} knockout mice (squares), and (±)-44-treated Abca4^{-/-} mice (triangles) after 12 weeks of dosing and analyzed by HPLC. A significant 50% reduction in A2E concentration was detected in (±)-44-treated Abca4^{-/-} mice compared with vehicle-treated knockout controls; one-way ANOVA with Holm-Sidak post hoc test, p<0.0001 (****).

FIG. 9. Effect of the long-term oral (±)-44 administration in Abca4^{-/-}/Rdh8^{-/-} mice on serum RBP4 levels. Serum RBP4 levels were measured in vehicle-treated Abca4^{-/-}/Rdh8^{-/-} mice (squares) and (±)-44-treated Abca4^{-/-}/Rdh8^{-/-} mice (triangles) at the indicated time points. Compared with the baseline, statistically significant 76% RBP4 reduction at week 10 was seen in the (±)-44 treatment group (two-way ANOVA with Sidak post hoc test, p<0.01). Error bars show S.D. Each data point on the graph represents a serum RBP4 concentration from an individual animal.

FIG. 10. Effect of the long-term oral (±)-44 administration on the level of the lipofuscin fluorophore A2E in eyes of the Abca4^{-/-}/Rdh8^{-/-} mice. Bisretinoids were extracted from the eyecups of vehicle-treated wild type mice (circles), vehicle-treated Abca4^{-/-}/Rdh8^{-/-} knockout mice (squares), and (±)-44-treated Abca4^{-/-}/Rdh8^{-/-} mice (triangles) after 10 weeks of dosing and analyzed by HPLC. A significant 77% reduction in A2E concentration was detected in (±)-44-treated Abca4^{-/-}/Rdh8^{-/-} mice compared with vehicle-treated double-knockout controls; one-way ANOVA with Holm-Sidak post hoc test, p<0.0001 (****).

FIG. 11. Photoreceptor protection in (±)-44-treated Rdh8^{-/-}Abca4^{-/-} mice. Reduction in the ONL thickness in retinas of the untreated Rdh8^{-/-}Abca4^{-/-} mice (squares) in comparison to the untreated wild type C57BL/6 mice (circles) is partially reversed by the (±)-44 treatment (triangles). Data analyzed by two-way ANOVA with Sidak post hoc test.

FIG. 12. Structure of bisretinoids A2E and isoA2E, cytotoxic components of retinal lipofuscin.

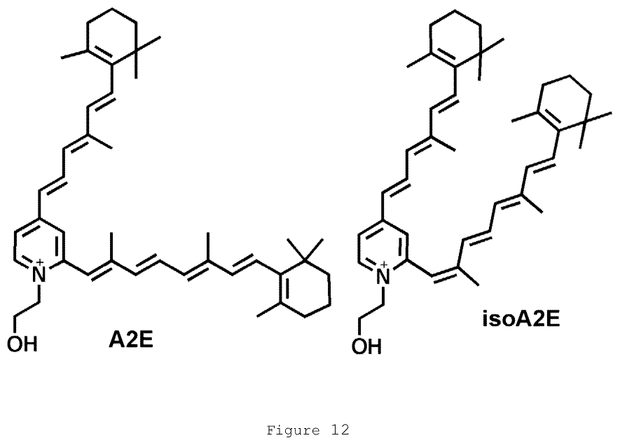

FIG. 13. Structure of bisretinoids atRAL di-PE (all-transretinal dimer-phosphatidyl ethanolamine) and A2-DHP-PE, cytotoxic components of retinal lipofuscin. R₁ and R₂ refer to various fatty acid constituents.

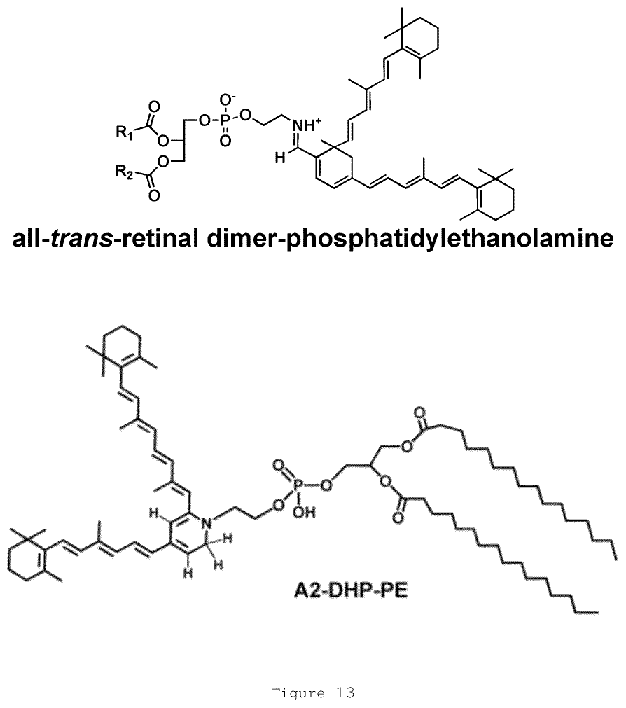

### DETAILED DESCRIPTION OF THE INVENTION

The present invention provides a compound having the structure:

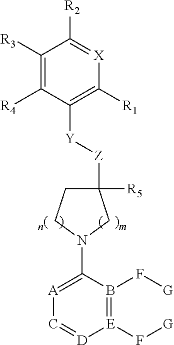

wherein
   X is CR₆ or N;
   R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈ or —NHC(S) R₈,
wherein R₇ is, H, -(alkyl), —OH, —O(alkyl), —NH₂, —NH(alkyl) or —N(alkyl)₂, and
wherein R₈ is, -(alkyl), —O-(alkyl), —NH₂, —NH(alkyl) or —N(alkyl)₂;
   Y is O, S, N, NH, or a bond;
   Z is O, S, N, NH, (CH₂)ₒ, or a bond;
   R₅ is H, OH, halogen, alkyl, or R₅ is (CH₂)ₚ and is bound to Y when Y is N to form a ring together with Z;
   and p are independently 0, 1, 2, or 3;
   m and n are independently 0, 1, 2, 3, or 4;
   A, C, and D are each independently N or CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, —CO(O-alkyl);
B and E are N, CR₉, or CFG wherein at least one of B or E is CFG;
   F is absent or present, and when present is

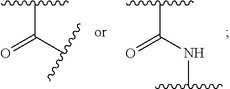

G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein each R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl), —N(alkyl)₂,
or a pharmaceutically acceptable salt thereof.

In some embodiments, the compound wherein m is 1 or 2 and n is 0, 1, or 2.

In some embodiments, the compound wherein m is 1 and n is 1.

In some embodiments, the compound wherein Y and Z are each independently CH₂, O, S, or NH.

In some embodiments, the compound wherein Y is O and Z is CH₂.

In some embodiments, the compound wherein A and B are N, C and D are CR₉, and E is CFG.

In some embodiments, the compound wherein A, B, C and D are CR₉, and E is CFG.

In some embodiments, the compound wherein A is N, B is CFG, C, D and E are each CR₉.

In some embodiments, the compound wherein A is N, B, C, and D are each CR₉ and E is CFG.

In some embodiments, the compound wherein A, C, and D are each CR₉, B is N, and E is CFG.

In some embodiments, the compound wherein wherein
X is CR₆ or N;
R₁, R₂, R₃, R₄, and R₆ are each independently H, t-butyl, cyclopently, cyclohexyl, CF₃, F, Cl, CN, or —OCH₃.

In some embodiments, the compound wherein R₁ or R₄ is CF₃.

In some embodiments, the compound wherein
X is CR₆
R₁ is CF₃ and R₂, R₃, R₄ and R₆ are each independently H, t-butyl, cyclopently, cyclohexyl, CF₃, F, Cl, CN, or —OCH₃.

In some embodiments, the compound having the structure:

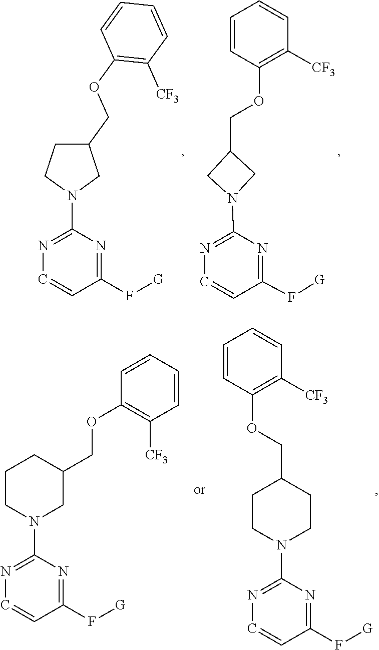

wherein
C is CR₉;
R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, —CO(O-alkyl);
F is absent or present, and when present is

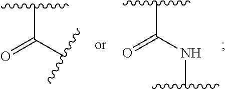

G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein each R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl), —N(alkyl)₂,

or a pharmaceutically acceptable salt thereof.

In some embodiments, the compound wherein C is CR₉ and
R₉ is H, alkyl, —O(alkyl) or —NH(alkyl)

In some embodiments, the compound wherein R₉ is -alkyl

In some embodiments, the compound having the structure:

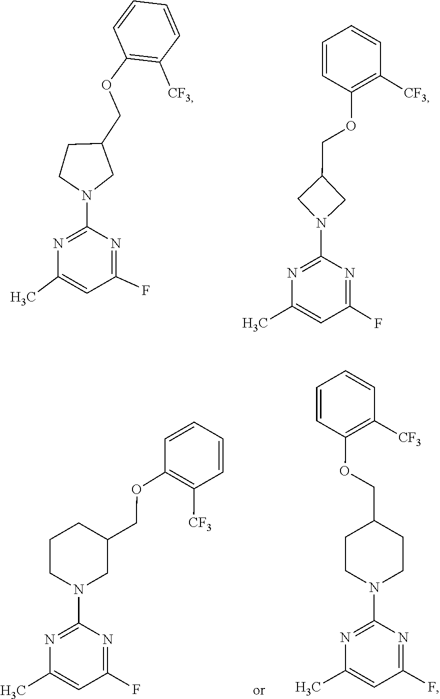

wherein
F is substituted or unsubstituted heteroaryl group,

In some embodiments, the compound wherein F has the structure:

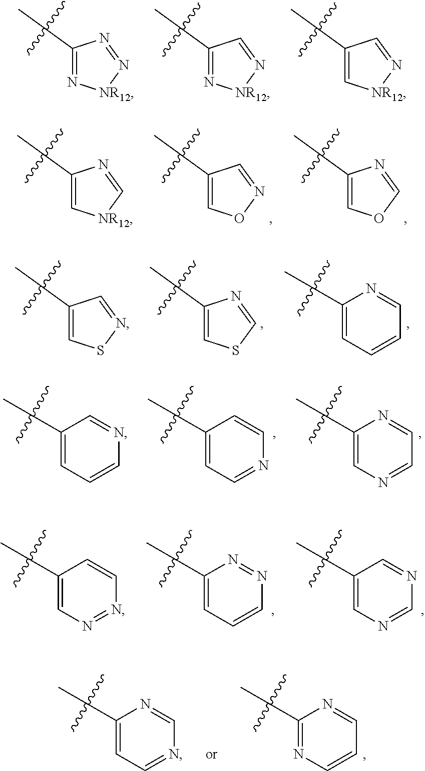

wherein R₁₂ is H, -(alkyl), -(alkenyl), -(alkynyl).

In some embodiments, the compound having the structure:

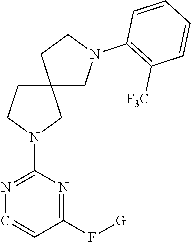

wherein
C is CR₉;
R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, —CO(O-alkyl);
F is absent or present, and when present is

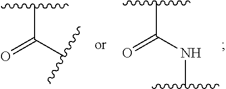

G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein each R₁₀ and R₁₁ are each independently H, alkyl, cycloalkyl, —C(O)-alkyl, —C(O)-cycloalkyl, —C(O)OH, —C(O)—O— alkyl, —C(O)—O-cycloalkyl, —C(O)NH₂, —C(O)NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl), —N(alkyl)₂, or a pharmaceutically acceptable salt thereof.
   In some embodiments, the compound wherein C is CR₉ and
R₉ is H, alkyl, —O(alkyl) or —NH(alkyl).

In some embodiments, the compound wherein R₉ is -alkyl.

In some embodiments, the compound having the structure:

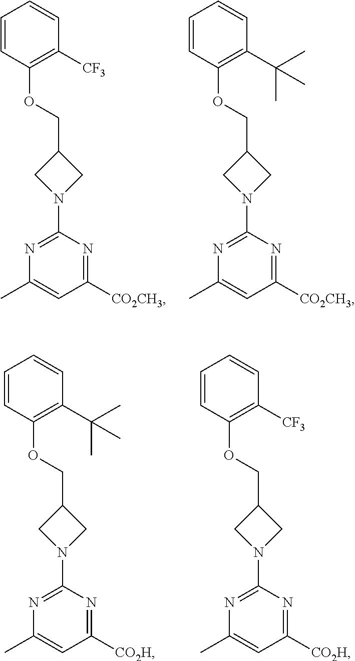 [SMILES: COC(=O)c1cc(C)nc(N2CC(COc3ccccc3C(F)(F)F)C2)n1 ; COC(=O)c1cc(C)nc(N2CC(COc3ccccc3C(C)(C)C)C2)n1 ; Cc1cc(C(=O)O)nc(N2CC(COc3ccccc3C(C)(C)C)C2)n1 ; Cc1cc(C(=O)O)nc(N2CC(COc3ccccc3C(F)(F)F)C2)n1]

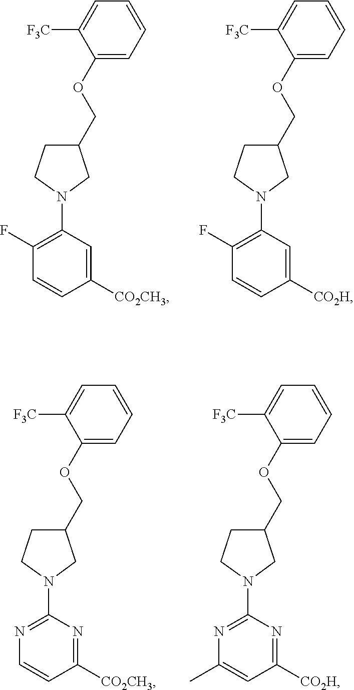 [SMILES: COC(=O)c1ccc(F)c(N2CCC(COc3ccccc3C(F)(F)F)C2)c1 ; O=C(O)c1ccc(F)c(N2CCC(COc3ccccc3C(F)(F)F)C2)c1 ; COC(=O)c1ccnc(N2CCC(COc3ccccc3C(F)(F)F)C2)n1 ; Cc1cc(C(=O)O)nc(N2CCC(COc3ccccc3C(F)(F)F)C2)n1]

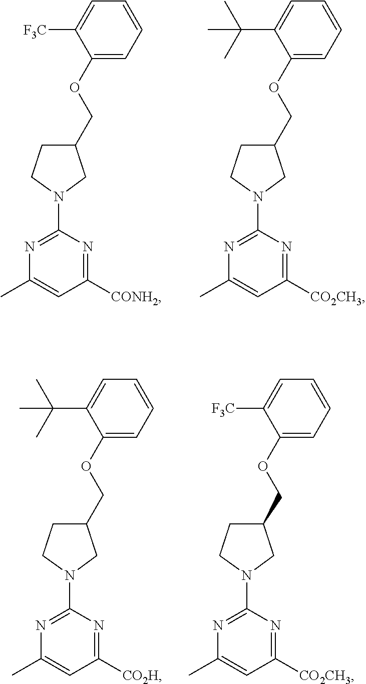 [SMILES: Cc1cc(C(N)=O)nc(N2CCC(COc3ccccc3C(F)(F)F)C2)n1 ; COC(=O)c1cc(C)nc(N2CCC(COc3ccccc3C(C)(C)C)C2)n1 ; Cc1cc(C(=O)O)nc(N2CCC(COc3ccccc3C(C)(C)C)C2)n1 ; COC(=O)c1cc(C)nc(N2CC[C@@H](COc3ccccc3C(F)(F)F)C2)n1]

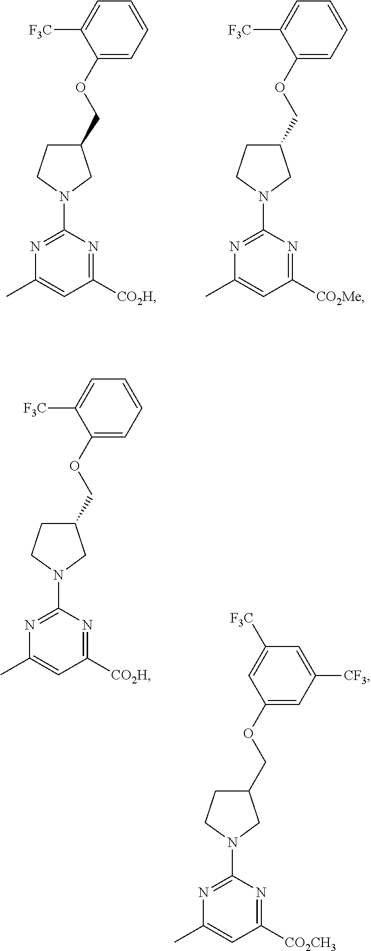 [SMILES: Cc1cc(C(=O)O)nc(N2CC[C@@H](COc3ccccc3C(F)(F)F)C2)n1 ; COC(=O)c1cc(C)nc(N2CC[C@H](COc3ccccc3C(F)(F)F)C2)n1 ; Cc1cc(C(=O)O)nc(N2CC[C@H](COc3ccccc3C(F)(F)F)C2)n1 ; COC(=O)c1cc(C)nc(N2CCC(COc3cc(C(F)(F)F)cc(C(F)(F)F)c3)C2)n1]

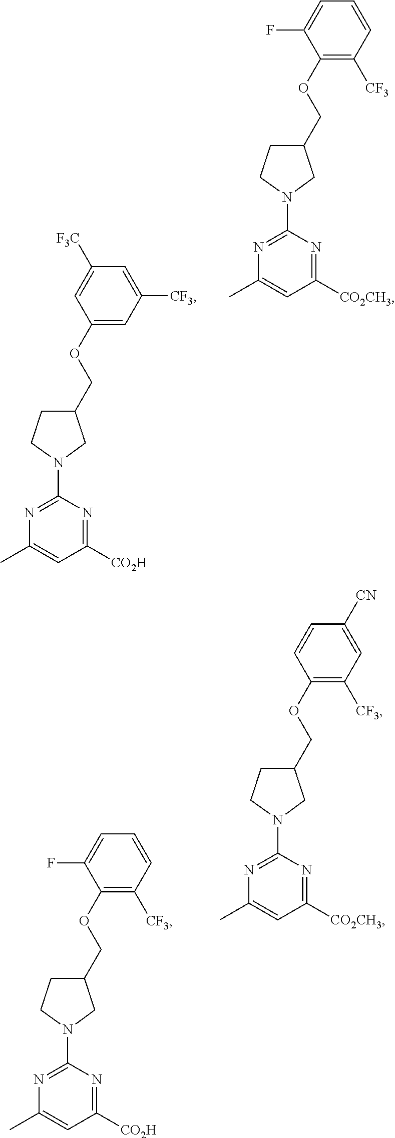 [SMILES: Cc1cc(C(=O)O)nc(N2CCC(COc3cc(C(F)(F)F)cc(C(F)(F)F)c3)C2)n1 ; COC(=O)c1cc(C)nc(N2CCC(COc3c(F)cccc3C(F)(F)F)C2)n1 ; Cc1cc(C(=O)O)nc(N2CCC(COc3c(F)cccc3C(F)(F)F)C2)n1 ; COC(=O)c1cc(C)nc(N2CCC(COc3ccc(C#N)cc3C(F)(F)F)C2)n1]

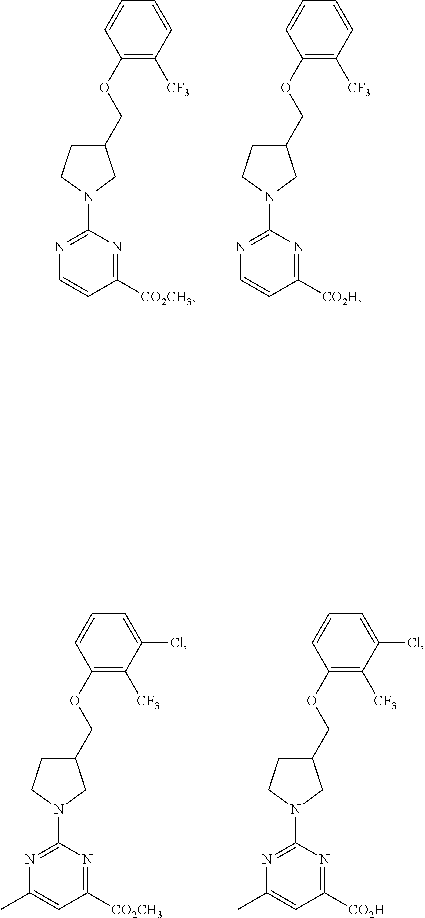 [SMILES: COC(=O)c1ccnc(N2CCC(COc3ccccc3C(F)(F)F)C2)n1 ; O=C(O)c1ccnc(N2CCC(COc3ccccc3C(F)(F)F)C2)n1 ; COC(=O)c1cc(C)nc(N2CCC(COc3cccc(Cl)c3C(F)(F)F)C2)n1 ; Cc1cc(C(=O)O)nc(N2CCC(COc3cccc(Cl)c3C(F)(F)F)C2)n1]

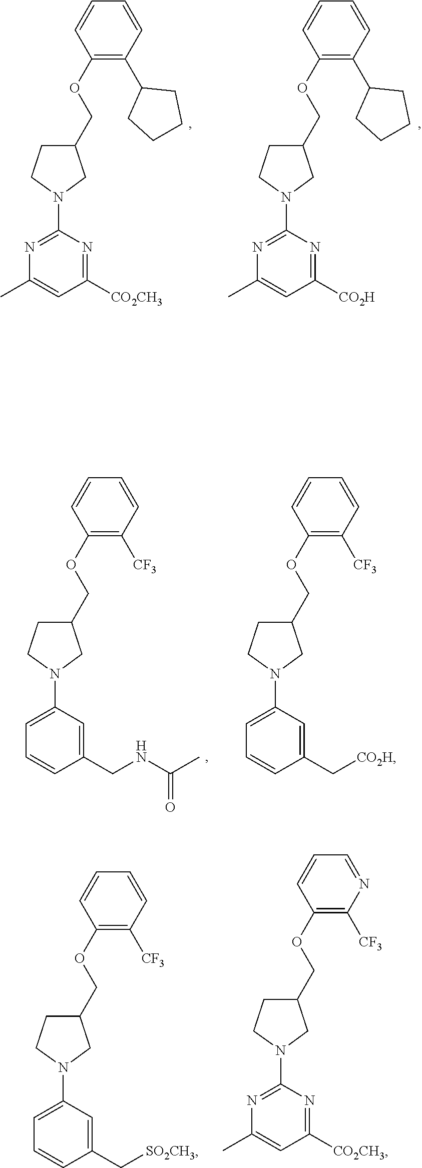 [SMILES: COC(=O)c1cc(C)nc(N2CCC(COc3ccccc3C3CCCC3)C2)n1 ; Cc1cc(C(=O)O)nc(N2CCC(COc3ccccc3C3CCCC3)C2)n1 ; CC(=O)NCc1cccc(N2CCC(COc3ccccc3C(F)(F)F)C2)c1 ; O=C(O)Cc1cccc(N2CCC(COc3ccccc3C(F)(F)F)C2)c1 ; CS(=O)(=O)Cc1cccc(N2CCC(COc3ccccc3C(F)(F)F)C2)c1 ; COC(=O)c1cc(C)nc(N2CCC(COc3cccnc3C(F)(F)F)C2)n1]

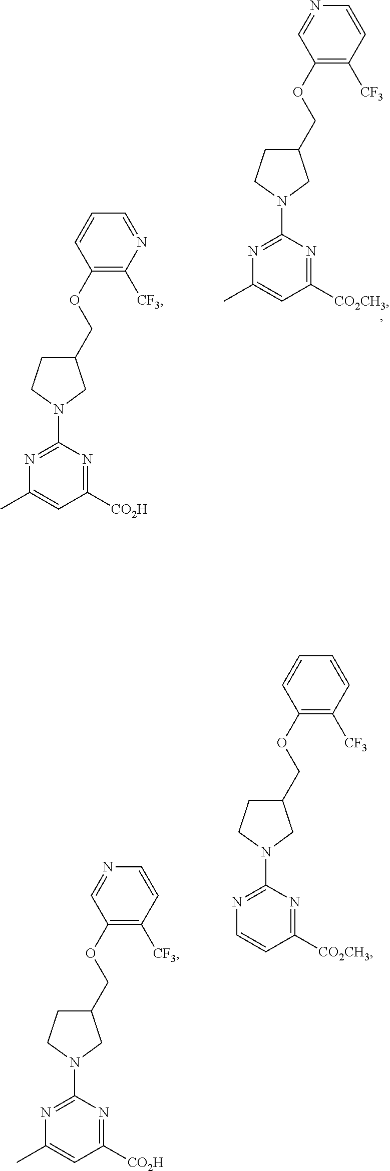 [SMILES: Cc1cc(C(=O)O)nc(N2CCC(COc3cccnc3C(F)(F)F)C2)n1 ; COC(=O)c1cc(C)nc(N2CCC(COc3cnccc3C(F)(F)F)C2)n1 ; Cc1cc(C(=O)O)nc(N2CCC(COc3cnccc3C(F)(F)F)C2)n1 ; COC(=O)c1ccnc(N2CCC(COc3ccccc3C(F)(F)F)C2)n1]

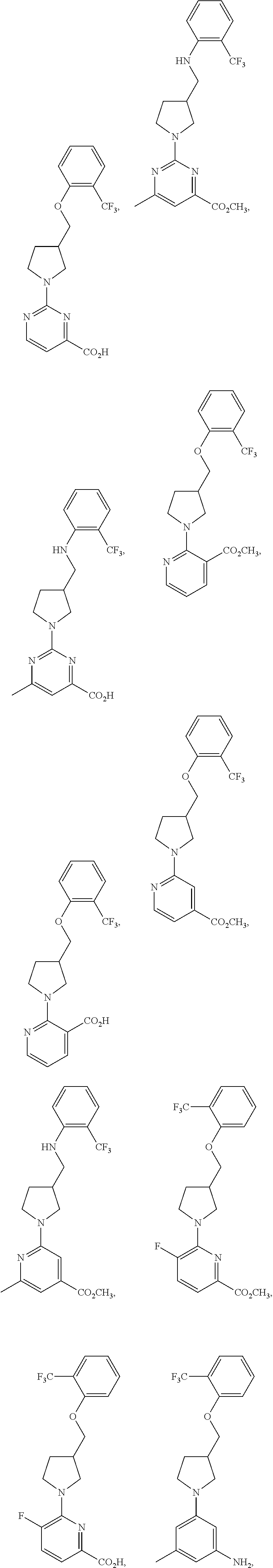

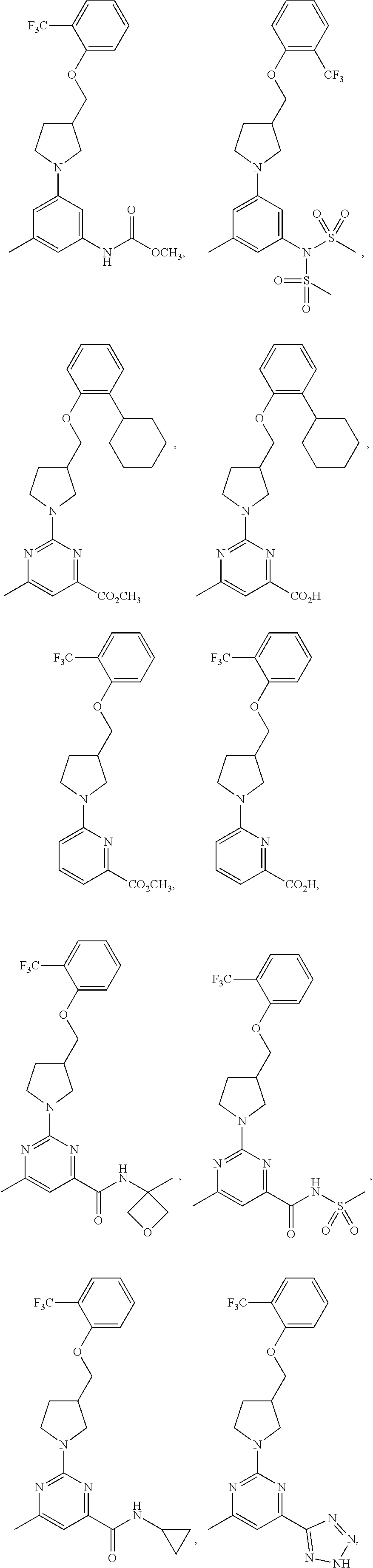

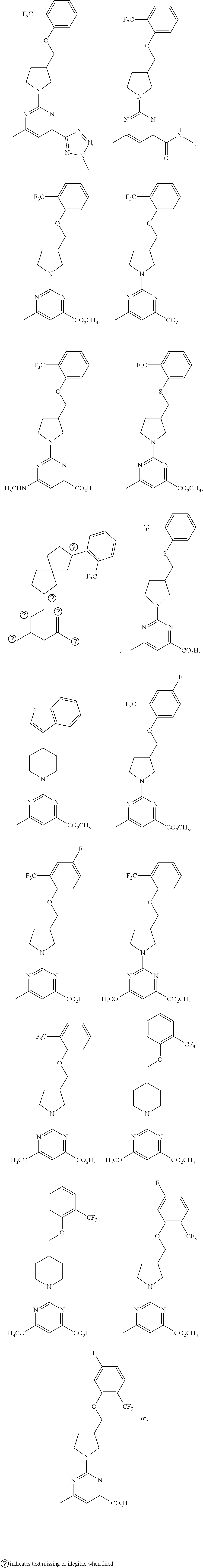

or a pharmaceutically acceptable salt of the compound.

In some embodiments, the compound having the structure:

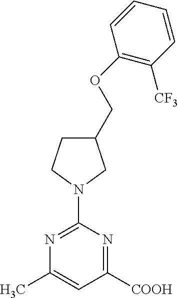 [SMILES: Cc1cc(C(=O)O)nc(N2CCC(COc3ccccc3C(F)(F)F)C2)n1]

or a pharmaceutically acceptable salt of the compound.

In some embodiments, the compound having the structure:

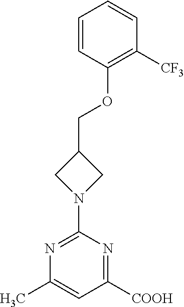 [SMILES: Cc1cc(C(=O)O)nc(N2CC(COc3ccccc3C(F)(F)F)C2)n1]

or a pharmaceutically acceptable salt of the compound.

In some embodiments, the compound having the structure:

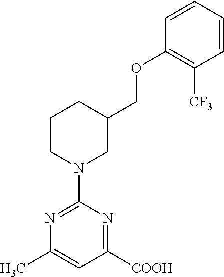 [SMILES: Cc1cc(C(=O)O)nc(N2CCCC(COc3ccccc3C(F)(F)F)C2)n1]

or a pharmaceutically acceptable salt of the compound.

In some embodiments, the compound having the structure:

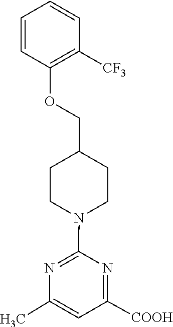 [SMILES: Cc1cc(C(=O)O)nc(N2CCC(COc3ccccc3C(F)(F)F)CC2)n1]

or a pharmaceutically acceptable salt of the compound.

In some embodiments, the compound having the structure:

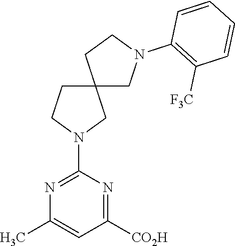 [SMILES: Cc1cc(C(=O)O)nc(N2CCC3(CCN(c4ccccc4C(F)(F)F)C3)C2)n1]

or a pharmaceutically acceptable salt of the compound.

The present invention provides a pharmaceutical composition comprising a compound of the present invention and a pharmaceutically acceptable carrier.

The present invention provides a method for stabilizing TTR tetramers in a mammal comprising administering to the mammal an effective amount of a compound of the present invention or a composition of the present invention so as to effectively stabilize TTR tetramers in a mammal.

The present invention provides a method for treating a disease characterized by excessive lipofuscin accumulation in the retina, or a TTR amyloidosis (ATTR) disease, or both a disease characterized by excessive lipofuscin and a TTR amyloidosis (ATTR) disease, in a mammal afflicted therewith comprising administering to the mammal an effective amount of a compound of the present invention or a composition of the present invention.

In some embodiments of the method, wherein the disease is further characterized by bisretinoid-mediated macular degeneration.

In some embodiments of the method, wherein the amount of the compound is effective to lower the serum concentration of RBP4 in the mammal, or wherein the amount of the compound is effective to lower the retinal concentration of a bisretinoid in lipofuscin in the mammal.

In some embodiments of the method, wherein the amount of the compound is effective to stabilize TTR tetramers in the mammal.

In some embodiments of the method, wherein the bisretinoid is A2E.

In some embodiments of the method, wherein the bisretinoid is isoA2E.

In some embodiments of the method, wherein the bisretinoid is A2-DHP-PE.

In some embodiments of the method, wherein the bisretinoid is atRAL di-PE.

In some embodiments of the method, wherein the disease characterized by excessive lipofuscin accumulation in the retina is Age-Related Macular Degeneration.

In some embodiments of the method, wherein the disease characterized by excessive lipofuscin accumulation in the retina is dry (atrophic) Age-Related Macular Degeneration.

In some embodiments of the method, wherein the disease characterized by excessive lipofuscin accumulation in the retina is Stargardt Disease.

In some embodiments of the method, wherein the disease characterized by excessive lipofuscin accumulation in the retina is Best disease.

In some embodiments of the method, wherein the disease characterized by excessive lipofuscin accumulation in the retina is adult vitelliform maculopathy.

In some embodiments of the method, wherein the disease characterized by excessive lipofuscin accumulation in the retina is Stargardt-like macular dystrophy.

In some embodiments of the method, wherein the administration is effective to reduce photoreceptor degeneration.

In some embodiments of the method, wherein the method is further effective to stabilize TTR tetramers in the mammal.

In some embodiments of the method, wherein the mammal is further afflicted with a TTR amyloidosis (ATTR) disease and the method is effective for treating the TTR amyloidosis (ATTR) disease in the mammal.

In some embodiments of the method, wherein the TTR amyloidosis (ATTR) disease is senile systemic amyloidosis (SSA).

In some embodiments of the method, wherein the TTR amyloidosis (ATTR) disease is peripheral polyneuropathy (ATTR-PN).

In some embodiments of the method, wherein the TTR amyloidosis (ATTR) disease is cardiomyopathy (ATTR-CM).

In some embodiments of the method, wherein the TTR amyloidosis (ATTR) disease is characterized by deposition of amyloid aggregates.

In some embodiments, bisretinoid-mediated macular degeneration is Age-Related Macular Degeneration or Stargardt Disease.

In some embodiments, the bisretinoid-mediated macular degeneration is Age-Related Macular Degeneration.

In some embodiments, the bisretinoid-mediated macular degeneration is dry (atrophic) Age-Related Macular Degeneration.

In some embodiments, the bisretinoid-mediated macular degeneration is Stargardt Disease.

In some embodiments, the bisretinoid-mediated macular degeneration is Best disease.

In some embodiments, the bisretinoid-mediated macular degeneration is adult vitelliform maculopathy.

In some embodiments, the bisretinoid-mediated macular degeneration is Stargardt-like macular dystrophy.

The bisretinoid-mediated macular degeneration may comprise the accumulation of lipofuscin deposits in the retinal pigment epithelium.

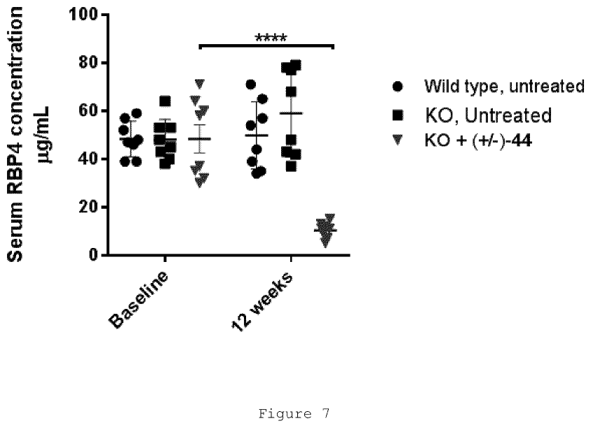

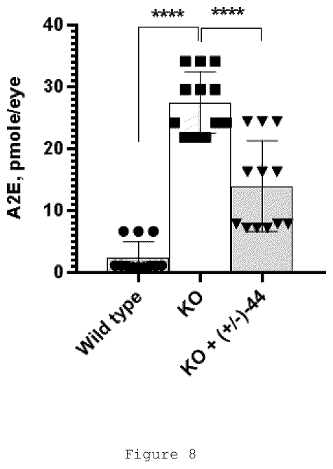

As used herein, “bisretinoid lipofuscin” is lipofuscin containing a cytotoxic bisretinoid. Cytotoxic bisretinoids include but are not necessarily limited to A2E, isoA2E, atRAL di-PE (all-trans-retinal dimer-phosphatidylethanolamine), and A2-DHP-PE (A2-dihydropyridine-phosphatidylethanolamine) (FIGS. 7-8).

Transthyretin (TTR) amyloidosis (ATTR) is a neurodegenerative disease and includes, but is not limited to, senile systemic amyloidosis (SSA), peripheral polyneuropathy (ATTR-PN), or cardiomyopathy (ATTR-CM).

In some embodiments, TTR amyloidosis (ATTR) diseases are characterized by the deposition of amyloid aggregates.

In some embodiments, TTR amyloidosis (ATTR) diseases are characterized by the deposition of amyloid aggregates derived from either mutant (TTRm) or wild-type (TTRwt).

In some embodiments, TTR amyloidosis (ATTR) disease is senile systemic amyloidosis (SSA).

In some embodiments, TTR amyloidosis (ATTR) disease is peripheral polyneuropathy (ATTR-PN).

In some embodiments, TTR amyloidosis (ATTR) disease is cardiomyopathy (ATTR-CM).

In some embodiments, the compounds of the present invention exhibit dual retinol-binding protein 4 (RBP4) antagonist and transthyretin (TTR) tetramer kinetic stabilization activity.

In some embodiments, the compounds of the present invention exhibit retinol-binding protein 4 (RBP4) antagonist activity.

In some embodiments, the compounds of the present invention exhibit transthyretin (TTR) tetramer kinetic stabilization activity.

In some embodiments, the compounds of the present invention reduce circulating RBP4 levels while simultaneously stabilizing unliganded TTR tetramers released from the holo-RBP4-TTR complex.

In some embodiments, the compounds of the present invention reduce circulating RBP4 levels.

In some embodiments, the compounds of the present invention stabilize unliganded TTR tetramers released from the holo-RBP4-TTR complex.

In some embodiments, the compounds of the present invention or composition of the present invention may be used for the treatment of dry age-related macular degeneration (AND) and TTR amyloidosis (ATTR) comorbidities.

In some embodiments, the compounds of the present invention or composition of the present invention may be used for the treatment of dry age-related macular degeneration (AND) and senile systemic amyloidosis (SSA).

In some embodiments, the compounds of the present invention or composition of the present invention may be used for the treatment of dry age-related macular degeneration (AMD) and peripheral polyneuropathy (ATTR-PN).

In some embodiments, the compounds of the present invention or composition of the present invention may be used for the treatment of dry age-related macular degeneration (AMD) and cardiomyopathy (ATTR-CM).

In some embodiments, the compounds of the present invention or composition of the present invention may be used for the treatment of type 2 diabetes.

In some embodiments, the compounds of the present invention or composition of the present invention may be used for the treatment of obesity.

In some embodiments, the compounds of the present invention or composition of the present invention may be used for the treatment of insulin resistance.

In some embodiments, the compounds of the present invention or composition of the present invention may be used for the treatment of cardiovascular disease.

In some embodiments, the compounds of the present invention or composition of the present invention may be used for the treatment of hepatic steatosis.

In some embodiments, the compounds of the present invention or composition of the present invention may be used for the treatment of non-alcoholic fatty liver disease (NAFLD).

In some embodiments, the mammal is a human.

A person skilled in the art may use the techniques disclosed herein to prepare deuterium analogs thereof.

Except where otherwise specified, the structure of a compound of this invention includes an asymmetric carbon atom, it is understood that the compound occurs as a racemate, racemic mixture, scalemic mixtures and isolated single enantiomers. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

Except where otherwise specified, the subject invention is intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notations of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notations of a hydrogen (H) in structures throughout this application, when used without further notation, are intended to represent all isotopes of hydrogen, such as ¹H, ²H (D), or ³H (T) except where otherwise specified. Furthermore, any compounds containing ²H or ³H may specifically have the structure of any of the compounds disclosed herein except where otherwise specified.

Isotopically labeled compounds can generally be prepared by conventional techniques known to those skilled in the art using appropriate isotopically labeled reagents in place of the non-labeled reagents employed.

Deuterium (²H or D) is a stable, non-radioactive isotope of hydrogen and has an atomic weight of 2.0144. Hydrogen atom in a compound naturally occurs as a mixture of the isotopes ¹H (hydrogen or protium), D (²H or deuterium), and T (³H or tritium). The natural abundance of deuterium is 0.0156%. Thus, in a composition comprising molecules of a naturally occurring compound, the level of deuterium at a particular hydrogen atom site in that compound is expected to be 0.0156%. Thus, a composition comprising a compound with a level of deuterium at any site of hydrogen atom in the compound that has been enriched to be greater than its natural abundance of 0.0156% is novel over its naturally occurring counterpart.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

In the compounds used in the method of the present invention, the substituents may be substituted or unsubstituted, unless specifically defined otherwise.

In the compounds used in the method of the present invention, alkyl, haloalkyl, alkenyl, alkynyl, aryl, heteroaryl, cycloalkyl, cycloalkylalkyl, heteroalkyl, heterocycle, heterocycloalkyl, alkylheteroalkyl, alkylaryl, monocycle, bicycle, heteromonocycle, and heterobicycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n-1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, and hexyl. Unless otherwise specified contains one to ten carbons. Alkyl groups can be unsubstituted or substituted with one or more substituents, including but not limited to halogen, alkoxy, alkylthio, trifluoromethyl, difluoromethyl, methoxy, and hydroxyl. “Haloalkyl” includes any alkyl group containing at least one halogen atom.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl or C₂-C_{E} alkenyl.

The term “alkynyl” refers to a hydrocarbon radical straight or branched, containing at least 1 carbon-to-carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated.

An embodiment can be a C₂-Cₙ alkynyl. An embodiment can be C₂-C₁₂ alkynyl or C₃-C₈ alkynyl.

As used herein, “aryl” is intended to mean any stable monocyclic, bicyclic, or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl or cyclooctyl). “Cycloalkylalkyl” includes any alkyl group containing at least one cycloalkyl ring.

As used herein, “heteroalkyl” includes both branched and straight-chain saturated aliphatic hydrocarbon groups having at least 1 heteroatom within the chain or branch. “Alkylheteroalkyl” includes any alkyl group containing at least one heteroalkyl group.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

As used herein, “heterocycloalkyl” is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains nitrogen, it is understood that the corresponding N-oxides thereof are also encompassed by this definition.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

As used herein, “monocycle” includes any stable polycyclic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl. As used herein, “heteromonocycle” includes any monocycle containing at least one heteroatom.

As used herein, “bicycle” includes any stable polycyclic carbon ring of up to 10 atoms that is fused to a polycyclic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene. As used herein, “heterobicycle” includes any bicycle containing at least one heteroatom.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the invention comprises a compound or composition of the present invention as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department of Health and Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat a disease or medical disorder, the salt is pharmaceutically acceptable. Examples of pharmaceutically acceptable salts include, but are not limited to, mineral or organic acid salts of basic residues such as amines; alkali or organic salts of acidic residues such as phenols; alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the sodium, potassium, or lithium salts, and the like. Carboxylate salts are the sodium, potassium, or lithium salts, and the like. The term “pharmaceutically acceptable salt” in this respect, refers to the relatively non-toxic, inorganic, and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

Aa salt or pharmaceutically acceptable salt is contemplated for all compounds disclosed herein.

As used herein, “treating” means preventing, slowing, halting, or reversing the progression of a disease. Treating may also mean improving one or more symptoms of a disease.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier, as are capsules, coatings, and various syringes.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of disease, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous, or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin, and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavoring and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al. 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, 17th ed., 1989, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention. Any of the disclosed generic or specific compounds may be applicable to any of the disclosed compositions, processes, or methods.

This invention will be better understood by reference to the Experimental Details which follow, but those skilled in the art will readily appreciate that the specific experiments detailed are only illustrative of the invention as described more fully in the claims, which follow thereafter.

### Experimental Details

### General Chemistry.

All reactions were performed under a dry atmosphere of nitrogen unless otherwise specified. Indicated reaction temperatures refer to the reaction bath, while room temperature (rt) is noted as 25° C. Commercial grade reagents and anhydrous solvents were used as received from vendors and no attempts were made to purify or dry these components further. Removal of solvents under reduced pressure was accomplished with a Buchi rotary evaporator at approximately 28 mm Hg pressure using a Teflon-linked KNF vacuum pump. Thin layer chromatography was performed using 1″×3″ AnalTech No. 02521 silica gel plates with fluorescent indicator. Visualization of TLC plates was made by observation with either short wave UV light (254 nm lamp), 10% phosphomolybdic acid in ethanol or in iodine vapors. Preparative thin layer chromatography was performed using Analtech, 20×20 cm, 1000 micron preparative TLC plates. Flash column chromatography was carried out using a Teledyne Isco CombiFlash Companion Unit and a Biotage® Selekt System with Teledyne Isco RediSep Rf and Biotage Sfar silica gel columns. If needed, products were purified by reverse phase chromatography, using a Teledyne Isco CombiFlash Companion Unit and a Biotage® Selekt System with a RediSep Gold C18 reverse phase column. Proton NMR spectra were obtained on a 400 MHz Varian nuclear magnetic resonance spectrometer. Chemical shifts (P) are reported in parts per million (ppm) and coupling constant (J) values are given in Hz, with the following spectral pattern designations: s, singlet; d, doublet; t, triplet, q, quartet; dd, doublet of doublets; m, multiplet; br, broad. Tetramethylsilane was used as an internal reference. Any melting points provided are uncorrected and were obtained using a MEL-TEMP Electrothermal melting point apparatus. Mass spectroscopic analyses were performed using ESI ionization on a Waters AQUITY UPLC MS triple quadrapole mass spectrometer. High pressure liquid chromatography (HPLC) purity analysis was performed using a Waters Breeze2 HPLC system with a binary solvent system A and B using a gradient elusion [A, H₂O with 0.1% formic acid; B, CH₃CN with 0.1% formic acid] and flow rate=0.5 mL/min, with UV detection at 254 nm (system equipped with a photodiode array (PDA) detector). An ACQUITY UPLC BEH C18 column, 130 Å, 1.7 μm, 2.1 mm×50 mm was used. All final compounds tested for in vitro and in vivo biological testing were purified to ≥95% purity, and these purity levels were measured by both ¹H NMR and HPLC.

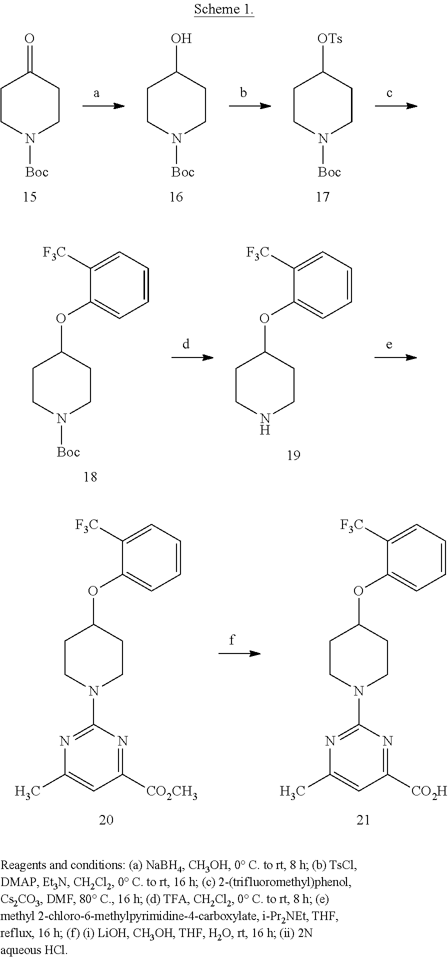

### Example 1: 6-Methyl-2-(4-(2-(trifluoromethyl)phenoxy)piperidin-1-yl)pyrimidine-4-carboxylic Acid 21

Step A: To a 0° C. cooled solution of tert-butyl 4-oxopiperidine-1-carboxylate 15 (5.0 g, 25.1 mmol) in CH₃OH (50 mL) at was added NaBH₄ (1.14 g, 30.1 mmol). The mixture stirred for 8 h while gradually warming to rt. The mixture was concentrated under reduced pressure and the resulting residue was diluted with H₂O (100 mL) and extracted with CH₂Cl₂ (2×100 mL). The combined organic extracts were dried over Na₂SO₄, filtered, and evaporated under reduced pressure to give tert-butyl 4-hydroxypiperidine-1-carboxylate 16 as a white solid (4.5 g, 89%): ¹H NMR (400 MHz, CDCl₃): δ 3.91-3.75 (m, 3H), 3.05-2.96 (m, 2H), 1.90-1.79 (m, 2H), 1.49-1.40 (m, 11H); ES MS: m/z 224 [M+Na]⁺.

Step B: To a 0° C. cooled solution of tert-butyl 4-hydroxypiperidine-1-carboxylate 16 (4.5 g, 22.3 mmol) in CH₂Cl₂ (50 mL) were added Et₃N (4.7 ml, 33.5 mmol) and DMAP (0.127 g, 1.10 mmol) followed by the addition of TsCl (5.10 g, 26.8 mmol). The resulting solution was stirred for 16 h while gradually warming to rt under an atmosphere of N₂. The mixture was diluted with saturated aqueous NaOH (50 mL) and extracted with EtOAc (3×100 mL). The combined organic extracts were washed with H₂O (100 mL), brine (100 mL), dried over Na₂SO₄, filtered, and concentrated under reduced pressure. The resulting residue was chromatographed over silica gel (0% to 50% EtOAc in hexanes) to give tert-butyl 4-(tosyloxy)piperidine-1-carboxylate 17 as a colorless liquid (6.6 g, 84%): ¹H NMR (400 MHz, CDCl₃) δ 7.82 (d, 2H), 7.49 (d, 2H), 4.69 (b. s, 1H), 3.49 (b. m, 2H), 3.15 (b. m, 2H), 2.43 (bs, 3H), 1.70 (b. m, 2H), 1.51 (b. m, 2H), 1.38 (s, 9H); ESI MS m/z 356 [M+H]+.

Step C: To a solution of tert-butyl 4-(tosyloxy)piperidine-1-carboxylate 17 (0.250 g, 0.703 mmol) in anhydrous DMF (4 mL) were added Cs₂CO₃ (0.450 g, 1.38 mmol) and 2-(trifluoromethyl)phenol (95.0 mg, 0.586 mmol) and the resulting solution was stirred at 80° C. for 16 h under an atmosphere of N₂. The mixture was allowed to cool to rt and then diluted with H₂O (20 mL). The aqueous mixture was extracted with EtOAc (3×25 mL) and the combined organic extracts were washed with H₂O (3×25 mL), brine (25 mL), dried over Na₂SO₄, filtered, and concentrated under reduced pressure. The resulting residue was chromatographed over silica gel (0% to 30% EtOAc in hexanes) to give tert-butyl 4-(2-(trifluoromethyl)phenoxy)piperidine-1-carboxylate 18 as a white solid (0.118 g, 56%): ¹H NMR (400 MHz, acetone-d₆) δ 7.65-7.55 (m, 2H), 7.30 (d, 1H), 7.08 (t, 1H), 4.92-4.82 (m, 1H), 3.67-3.57 (m, 2H), 3.50-3.40 (m, 2H), 2.0-1.90 (m, 2H), 1.80-1.70 (m, 2H), 1.45 (s, 9H); ESI MS m/z 346 [M+H]+.

Step D: To a 0° C. cooled solution of tert-butyl 4-(2-(trifluoromethyl)phenoxy)piperidine-1-carboxylate 18 (0.118 g, 0.341 mmol) in CH₂Cl₂ (10 mL) was added TFA (0.33 mL, 4.31 mmol) and the resulting solution was stirred for 16 h while gradually warming to rt. The mixture was neutralized by being carefully poured into a solution of saturated aqueous NaHCO₃ (10 mL). The biphasic mixture was separated and the aqueous layer was further extracted with CH₂Cl₂ (3×20 mL). The combined organic extracts were washed with brine (20 mL), dried over Na₂SO₄, filtered, and concentrated under reduced pressure to give 4-(2-(trifluoromethyl)phenoxy)piperidine 19 as a white solid (80.0 mg, 95%): ¹H NMR (400 MHz, acetone-d₆) δ 7.70-7.60 (m, 2H), 7.37 (d, 1H), 7.14 (t, 1H), 5.10-5.03 (m, 1H), 3.50-3.40 (m, 4H), 2.47-2.37 (m, 2H), 2.21-2.11 (m, 2H); ESI MS m/z 246 [M+H]+.

Step E: A mixture of 4-(2-(trifluoromethyl)phenoxy)piperidine 19 (0.100 g, 0.408 mmol), methyl 2-chloro-6-methylpyrimidine-4-carboxylate (76.1 mg, 0.408 mmol), and i-Pr₂NEt (0.21 mL, 1.22 mmol) in THF (10 mL) was heated at reflux for 16 h under an atmosphere of N₂. The reaction was concentrated under reduced pressure and the resulting residue was chromatographed over silica gel (0% to 100% EtOAc in hexanes) to give methyl 6-methyl-2-(4-(2-(trifluoromethyl)phenoxy)piperidin-1-yl)pyrimidine-4-carboxylate 20 as an off-white solid (0.140 g, 87%): MS (ESI+) m/z 396 [M+H]+.

Step F: A solution of methyl 6-methyl-2-(4-(2-(trifluoromethyl)phenoxy)piperidin-1-yl)pyrimidine-4-carboxylate 20 (0.100 g, 0.253 mmol) and LiOH (18.1 mg, 0.758 mmol) in CH₃OH (5 mL), THF (5 mL), and H₂O (5 mL) stirred at rt for 16 h. The mixture was acidified to pH=5 with 2 N aqueous HCl and extracted with CH₂Cl₂ (3×10 mL). The combined organic extracts were washed with brine (20 mL), dried over Na₂SO₄, filtered, and concentrated under reduced pressure to give 6-methyl-2-(4-(2-(trifluoromethyl)phenoxy)piperidin-1-yl)pyrimidine-4-carboxylic acid 21 as an off-white solid (87.8 mg, 91%): ¹H NMR (400 MHz, CDCl₃) δ 7.56-7.53 (m, 2H), 7.22 (d, J=6 Hz, 2H), 7.02-7.51 (m, 1H), 4.86 (brs, 1H), 3.99-3.85 (m, 4H), 2.37 (s, 3H), 2.05-1.95 (m, 2H), 1.81-1.80 (m, 2H); ESI MS m/z 382 [M+H]⁺; HPLC>99% (AUC), t_{R}=16.8 min.

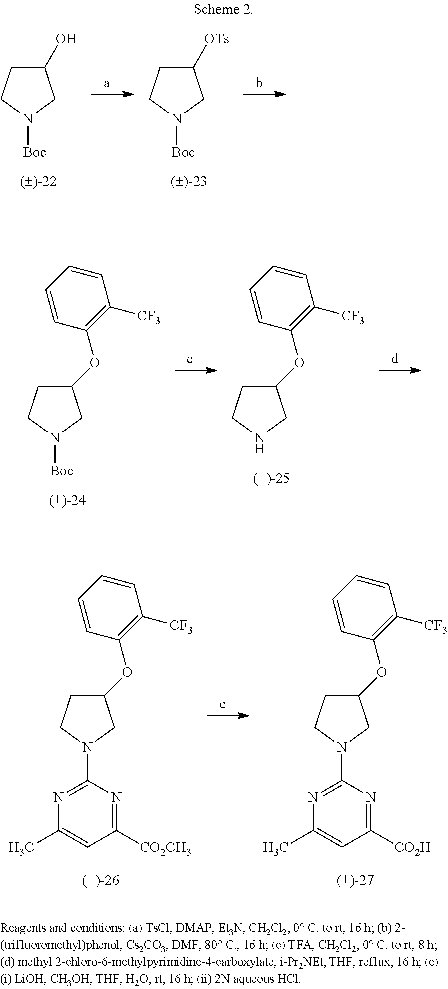 [SMILES: CC(C)(C)OC(=O)N1CCC(O)C1 ; Cc1ccc(S(=O)(=O)OC2CCN(C(=O)OC(C)(C)C)C2)cc1 ; CC(C)(C)OC(=O)N1CCC(Oc2ccccc2C(F)(F)F)C1 ; FC(F)(F)c1ccccc1OC1CCNC1 ; COC(=O)c1cc(C)nc(N2CCC(Oc3ccccc3C(F)(F)F)C2)n1 ; Cc1cc(C(=O)O)nc(N2CCC(Oc3ccccc3C(F)(F)F)C2)n1]

### Example 2: (±)-6-Methyl-2-(3-(2-(trifluoromethyl)phenoxy)pyrrolidin-1-yl)pyrimidine-4-carboxylic Acid (±)-27

Step A: To a 0° C. cooled solution of (±)-tert-butyl 3-hydroxypyrrolidine-1-carboxylate (±)-22 (1.00 g, 5.34 mmol) in CH₂Cl₂ (20 mL) were added Et₃N (1.1 mL, 8.02 mmol) and DMAP (32.0 mg, 0.262 mmol) followed by the addition of TsCl (1.10 g, 5.88 mmol). The resulting solution was stirred for 16 h while gradually warming to rt under an atmosphere of N2. The mixture was diluted with saturated aqueous NaOH (20 mL) and extracted with EtOAc (3×50 mL). The combined organic extracts were washed with H₂O (50 mL) and brine (50 mL), dried over Na₂SO₄, filtered, and concentrated under reduced pressure. The resulting residue was chromatographed over silica gel (0% to 50% EtOAc in hexanes) to give (±)-tert-butyl 3-(tosyloxy)pyrrolidine-1-carboxylate (±)-23 as a colorless liquid (1.50 g, 82%): ¹H NMR (400 MHz, CDCl₃) δ 7.81 (d, 8.9 Hz, 2H), 7.34 (d, 7.8 Hz, 2H), 5.05 (bs, 1H), 3.43 (m, 4H), 2.43 (bs, 3H), 2.06 (m, 2H), 1.45 (s, 9H); ESI MS m/z 342 [M+H]+.

Step B: To a solution of (±)-tert-butyl 3-(tosyloxy)pyrrolidine-1-carboxylate (±)-23 (0.100 g 0.293 mmol) in DMF (4 mL) were added Cs₂CO₃ (0.290 g, 0.902 mmol) and 2-(trifluoromethyl)phenol (73.0 mg, 0.450 mmol) and the resulting mixture was stirred at 80° C. for 16 h under an atmosphere of N₂. The mixture was allowed to cool to rt and then diluted with H₂O (20 mL). The aqueous mixture was extracted with EtOAc (3×25 mL) and the combined organic extracts were washed with H₂O (3×25 mL), brine (25 mL), dried over Na₂SO₄, filtered, and concentrated under reduced pressure. The resulting residue was chromatographed over silica gel (0% to 30% EtOAc in hexanes) to give (±)-tert-butyl 3-(2-(trifluoromethyl)phenoxy)pyrrolidine-1-carboxylate (±)-24 as a white solid (80.0 mg, 83%): ¹H NMR (400 MHz, acetone-d₆) δ 7.62 (m, 2H), 7.34 (d, 1H), 7.15 (t, 1H), 5.20 (s, 1H), 3.65-3.40 (m, 4H), 2.19 (m, 2H), 1.44 (s, 9H); ESI MS m/z 332 [M+H]+.

Step C: To a 0° C. cooled solution of (±)-tert-butyl 3-(2-(trifluoromethyl)phenoxy)pyrrolidine-1-carboxylate (±)-24 (80.0 mg, 0.241 mmol) in CH₂Cl₂ (5 mL) was added TFA (0.18 mL, 2.41 mmol) and the resulting solution was stirred at for 8 h while gradually warming to rt. The mixture was neutralized by carefully pouring it into a solution of saturated aqueous NaHCO₃ (10 mL). The biphasic mixture was separated and the aqueous layer was further extracted with CH₂Cl₂ (3×20 mL). The combined organic extracts were washed with brine (20 mL), dried over Na₂SO₄, filtered, and concentrated under reduced pressure to give (±)-3-(2-(trifluoromethyl)phenoxy)pyrrolidine (±)-25 as a white solid (50.0 mg, 90%); ESI MS m/z 232 [M+H]i.

Step D: A mixture of (±)-3-(2-(trifluoromethyl)phenoxy)pyrrolidine (±)-25 (0.100 g, 0.432 mmol), methyl 2-chloro-6-methylpyrimidine-4-carboxylate (80.6 mg, 0.432 mmol), and i-Pr₂NEt (0.23 mL, 1.29 mmol) in THF (10 mL) was heated at reflux for 16 h under an atmosphere of N₂. The reaction was allowed to cool to rt and then concentrated under reduced pressure. The resulting residue was chromatographed over silica gel (0% to 100% EtOAc in hexanes) to give (±)-methyl 6-methyl-2-(3-(2-(trifluoromethyl)phenoxy)pyrrolidin-1-yl)pyrimidine-4-carboxylate (±)-26 as an off-white solid (0.145 g, 88%): ¹H NMR (400 MHz, CDCl₃) δ 7.52 (d, J=8.0 Hz, 1H), 7.45 (t, J=7.6 Hz, 1H), 7.00 (s, 1H), 6.98-6.95 (m, 2H), 5.10 (s, 1H), 4.00-3.86 (m, 3H), 3.89 (s, 3H), 3.76-3.74 (m, 2H), 2.38 (s, 3H), 2.22-2.19 (m, 1H); ESI MS m/z 382 [M+H]⁺.

Step E: A solution of (±)-methyl 6-methyl-2-(3-(2-(trifluoromethyl)phenoxy)pyrrolidin-1-yl)pyrimidine-4-carboxylate (±)-26 (48.8 g, 0.128 mmol) and LiOH (9.21 mg, 0.384 mmol) in CH₃OH (5 mL), THF (5 mL), and H₂O (5 mL) was stirred at rt for 16 h. The mixture was acidified to pH 5 with 2 N aqueous HCl and extracted with CH₂Cl₂ (3×10 mL). The combined organic extracts were washed with brine, dried over Na₂SO₄, filtered, and concentrated under reduced pressure to give (±)-6-methyl-2-(3-(2-(trifluoromethyl)phenoxy)pyrrolidin-1-yl)pyrimidine-4-carboxylic acid (±)-27 as an off-white solid (0.043 g, 91%): ¹H NMR (400 MHz, DMSO-d₆) δ 7.61-7.54 (m, 2H), 7.34-7.33 (m, 1H), 7.00-6.93 (m, 2H), 5.31 (s, 1H), 3.77-3.86 (m, 3H), 3.50-3.48 (m, 1H), 2.30 (s, 3H), 2.29-2.20 (m, 2H); ESI MS m/z 368 [M+H]⁺; HPLC>99% (AUC), t_{R}=14.5 min.

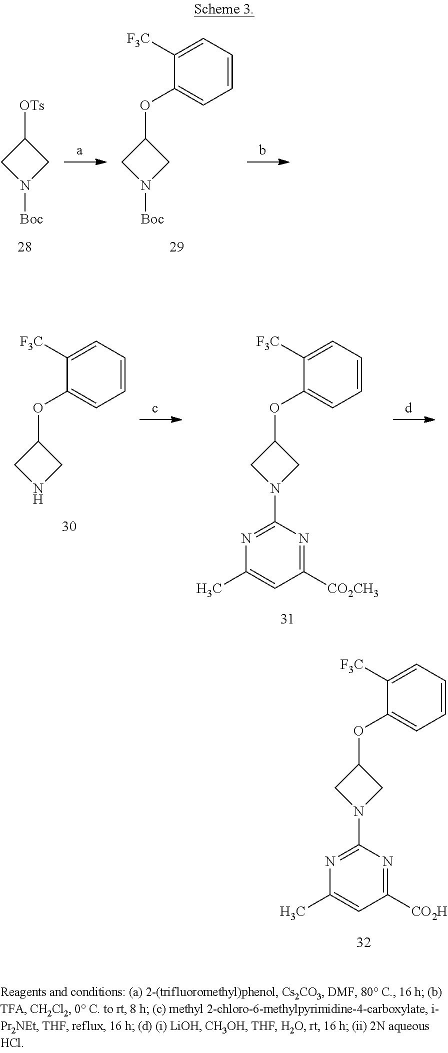 [SMILES: Cc1ccc(S(=O)(=O)OC2CN(C(=O)OC(C)(C)C)C2)cc1 ; CC(C)(C)OC(=O)N1CC(Oc2ccccc2C(F)(F)F)C1 ; FC(F)(F)c1ccccc1OC1CNC1 ; COC(=O)c1cc(C)nc(N2CC(Oc3ccccc3C(F)(F)F)C2)n1 ; Cc1cc(C(=O)O)nc(N2CC(Oc3ccccc3C(F)(F)F)C2)n1]

### Example 3: 6-Methyl-2-(3-(2-(trifluoromethyl)phenoxy)azetidin-1-yl)pyrimidine-4-carboxylic Acid 32

Step A: To a solution of tert-butyl 3-(tosyloxy)azetidine-1-carboxylate 28 (0.500 g 1.52 mmol) in DMF (20 mL) were added Cs₂CO₃ (990 mg, 3.05 mmol) and 2-(trifluoromethyl)phenol (0.272 g, 1.68 mmol) and the resulting solution was stirred at 80° C. for 16 h under an atmosphere of N₂. The mixture was allowed to cool to rt and then diluted with H₂O (50 mL) and extracted with EtOAc (3×50 mL). The combined organic extracts were washed with H₂O (3×50 mL), brine, dried over Na₂SO₄, filtered, and concentrated under reduced pressure. The resulting residue was chromatographed over silica gel (0% to 30% EtOAc in hexanes) to give tert-butyl 3-(2-(trifluoromethyl)phenoxy)azetidine-1-carboxylate 29 as a colorless liquid (0.400 g, 82%): ¹H NMR (400 MHz, CDCl₃) δ 7.59 (d, J=7.3 Hz, 1H), 7.61-7.45 (m, 1H), 7.06-7.03 (m, 1H), 6.65 (d, J=7.9 Hz, 1H), 4.99-4.91 (m, 1H), 4.35-4.30 (m, 2H), 4.10-4.05 (m, 2H), 1.44 (s, 9H); ESI MS m/z 318 [M+H]⁺.

Step B: To a 0° C. cooled solution of tert-butyl 3-(2-(trifluoromethyl)phenoxy)azetidine-1-carboxylate 29 (0.400 g, 1.20 mmol) in CH₂Cl₂ (20 mL) was TFA (0.96 mL, 12.0 mmol) and the resulting solution was stirred for 16 h while gradually warming to rt. The mixture neutralized by carefully pouring it into a solution of saturated aqueous NaHCO₃ (10 mL). The biphasic mixture was separated and the aqueous layer was further extracted with CH₂Cl₂ (3×20 mL). The combined organic extracts were washed with brine (20 mL), dried over Na₂SO₄, filtered, and concentrated under reduced pressure to give 3-(2-(trifluoromethyl)phenoxy)azetidine 30 as a white solid (0.240 g, 87%): ESI MS m/z 218 [M+H]⁺.

Step C: A mixture of 3-(2-(trifluoromethyl)phenoxy)azetidine 30 (0.100 g, 0.460 mmol), methyl 2-chloro-6-methylpyrimidine-4-carboxylate (85.9 mg, 0.460 mmol), and i-Pr₂NEt (0.24 mL, 1.38 mmol) in THF (10 mL) was heated at reflux for 16 h under an atmosphere of N₂. The reaction was concentrated under reduced pressure and the resulting residue was chromatographed over silica gel (0% to 100% EtOAc in hexanes) to give methyl 6-methyl-2-(3-(2-(trifluoromethyl)phenoxy)azetidin-1-yl)pyrimidine-4-carboxylate 31 as an off-white solid (0.152 g, 90%): MS (ESI+) m/z [M+H]⁺.

Step D: A solution of methyl 6-methyl-2-(3-(2-(trifluoromethyl)phenoxy)azetidin-1-yl)pyrimidine-4-carboxylate 31 (0.100 g, 0.272 mmol) and LiOH (19.5 mg, 0.816 mmol) in CH₃OH (5 mL), THF (5 mL), and H₂O (5 mL) stirred at rt for 16 h. The mixture was acidified to pH 5 with 2 N aqueous HCl and extracted with CH₂Cl₂ (3×10 mL). The combined organic extracts were washed with brine (10 mL), dried over Na₂SO₄, filtered, and concentrated under reduced pressure to give 6-methyl-2-(3-(2-(trifluoromethyl)phenoxy)azetidin-1-yl)pyrimidine-4-carboxylic acid 32 as an off-white solid (83.6 mg, 87%): ¹H NMR (400 MHz, CDCl₃) δ 7.59-7.54 (m, 2H), 7.14 (s, 1H), 7.08 (t, J=7.6 Hz, 1H), 6.94 (d, J=8.0 Hz, 1H), 5.28 (m, 1H), 4.61-4.60 (m, 2H), 4.15-4.12 (m, 2H), 2.40 (s, 3H); ESI MS m/z 354 [M+H]⁺; ESI MS m/z 354 [M+H]⁺; HPLC>99% (AUC), t_{R}=14.1 min.

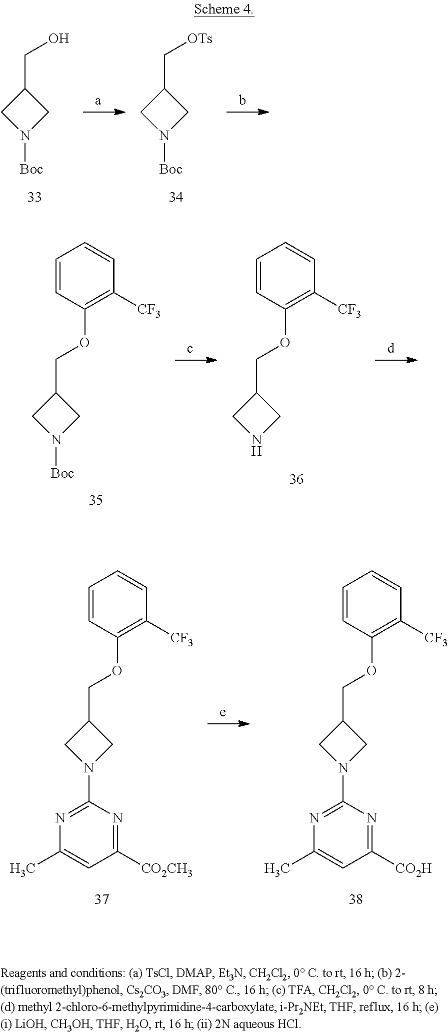 [SMILES: CC(C)(C)OC(=O)N1CC(CO)C1 ; Cc1ccc(S(=O)(=O)OCC2CN(C(=O)OC(C)(C)C)C2)cc1 ; CC(C)(C)OC(=O)N1CC(COc2ccccc2C(F)(F)F)C1 ; FC(F)(F)c1ccccc1OCC1CNC1 ; COC(=O)c1cc(C)nc(N2CC(COc3ccccc3C(F)(F)F)C2)n1 ; Cc1cc(C(=O)O)nc(N2CC(COc3ccccc3C(F)(F)F)C2)n1]

### Example 4: 6-Methyl-2-(3-((2-(trifluoromethyl)phenoxy)methyl)azetidin-1-yl)pyrimidine-4-carboxylic Acid 38

Step A: To a 0° C. cooled solution of tert-butyl 3-(hydroxymethyl)azetidine-1-carboxylate 33 (3.0 g, 16.0 mmol) in CH₂Cl₂ (50 mL) were added Et₃N (4.5 ml, 32.0 mmol) and DMAP (97.0 mg, 0.736 mmol) followed by the addition of TsCl (3.35 g, 17.6 mmol). The resulting solution was stirred for 16 h while gradually warming to rt under an atmosphere of N₂. The mixture was diluted with saturated solution of aqueous NaOH (50 mL) and was extracted with EtOAc (3×100 mL). The combined organic extracts were washed with H₂O (50 mL) and brine (50 mL), dried over Na₂SO₄, filtered, and concentrated under reduced pressure to give tert-butyl 3-((tosyloxy)methyl)azetidine-1-carboxylate 34 as a colorless liquid (5.0 g, 92%): ESI MS m/z 342 [M+H]⁺.

Step B: To a solution of tert-butyl 3-((tosyloxy)methyl)azetidine-1-carboxylate 34 (5.0 g 14.6 mmol) in DMF (50 mL) were added Cs₂CO₃ (9.5 g, 29.32 mmol) and 2-(trifluoromethyl)phenol (2.3 g, 14.6 mmol) and the resulting mixture was stirred at 80° C. for 16 h under an atmosphere of N₂. The mixture was allowed to cool to rt and then diluted with H₂O (100 mL) and extracted with EtOAc (3×50 mL). The combined organic extracts were washed with H₂O (3×50 mL), brine (50 mL), dried over Na₂SO₄, filtered, and concentrated under reduced pressure to give crude tert-butyl 3-((2-(trifluoromethyl)phenoxy)methyl)azetidine-1-carboxylate 35 as a brown liquid (4.5 g, 93%): ESI MS m/z 332 [M+H]⁺.

Step C: To a 0° C. cooled solution of tert-butyl 3-((2-(trifluoromethyl)phenoxy)methyl)azetidine-1-carboxylate 35 (4.50 g, 13.59 mmol) in CH₂Cl₂ (50 mL) was added TFA (10.3 mL, 135 mmol) and the resulting solution was stirred at for 8 h while gradually warming to rt. The mixture was neutralized by carefully pouring it into a solution of saturated aqueous NaHCO₃ (10 mL). The biphasic mixture was separated and the aqueous layer was further extracted with CH₂Cl₂ (3×20 mL). The combined organic extracts were washed with brine (20 mL), dried over Na₂SO₄, filtered, and concentrated under reduced pressure to give 3-((2-(trifluoromethyl)phenoxy)methyl)piperidine 36 as a white solid (2.8 g, 90% crude): ESI MS m/z 232 [M+H]⁺.

Step D: A mixture of 3-((2-(trifluoromethyl)phenoxy)methyl)azetidine 36 (1.0 g, 4.32 mmol), methyl 2-chloro-6-methylpyrimidine-4-carboxylate (0.807 g, 4.32 mmol), and i-Pr₂NEt (2.25 mL, 12.9 mmol) in THF (20 mL) was heated at reflux for 16 h under an atmosphere of N₂. The reaction was concentrated under reduced pressure and the resulting residue was chromatographed over silica gel (0% to 100% EtOAc in hexanes) to give methyl 6-methyl-2-(3-((2-(trifluoromethyl)phenoxy)methyl)azetidin-1-yl)pyrimidine-4-carboxylate 37 as an off-white solid (1.64 g, 86%): ¹H NMR (400 MHz, CDCl₃) δ 7.52 (d, J=7.6 Hz, 1H), 7.45 (t, J=7.6 Hz, 1H), 7.05 (s, 1H), 7.00-6.95 (m, 2H), 4.33 (t, J=8.8 Hz, 2H), 4.22-4.21 (m, 2H), 4.08-4.04 (m, 2H), 3.91 (s, 3H), 3.18-3.16 (m, 1H), 2.40 (s, 3H); ESI MS m/z 382 [M+H]⁺.

Step E: A solution of methyl 6-methyl-2-(3-((2-(trifluoromethyl)phenoxy)methyl)azetidin-1-yl)pyrimidine-4-carboxylate 37 (1.0 g, 2.62 mmol) and LiOH (0.188 g, 7.86 mmol) in CH₃OH (10 mL), THF (10 mL), and H₂O (10 mL) was stirred at rt for 16 h. The mixture was acidified to pH=5 with 2 N aqueous HCl and extracted with CH₂Cl₂ (3×10 mL). The combined organic extracts were washed with brine (10 mL), dried over Na₂SO₄, filtered, and concentrated under reduced pressure to give 6-methyl-2-(3-((2-(trifluoromethyl)phenoxy)methyl)azetidin-1-yl)pyrimidine-4-carboxylic acid 38 as an off-white solid (0.914 g, 95%): ¹H NMR (400 MHz, CDCl₃) δ 7.51 (d, J=7.6 Hz, 1H), 7.46 (t, J=7.6 Hz, 1H), 7.00 (s, 1H), 7.00-6.94 (m, 2H), 4.33 (t, J=8.4 Hz, 2H), 4.24-4.22 (m, 2H), 4.07-4.03 (m, 2H), 3.17-3.15 (m, 1H), 2.42 (s, 3H); ESI MS m/z 368 [M+H]⁺; HPLC 98.2% (AUC), t_{R}=13.5 min.

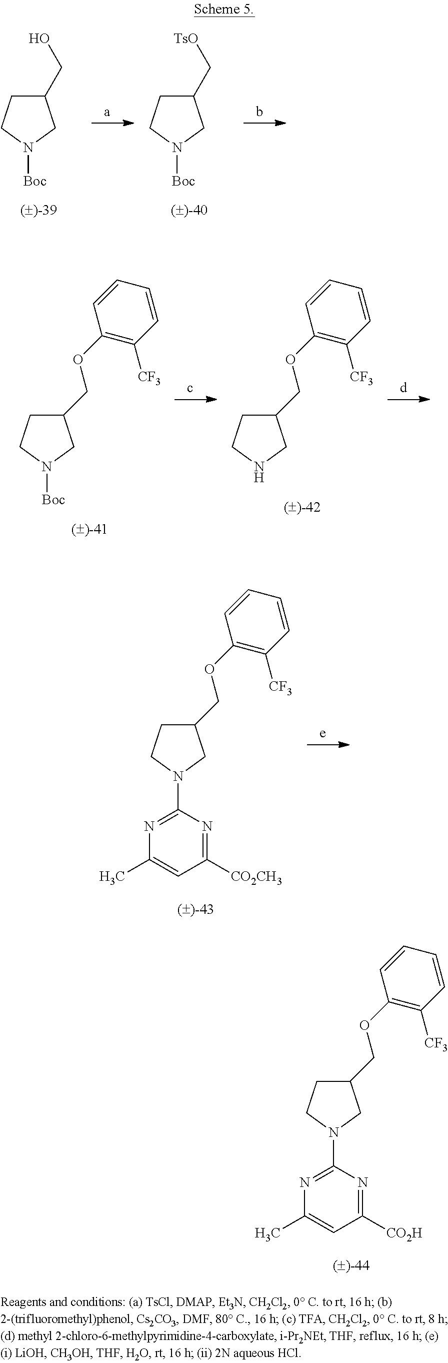 [SMILES: CC(C)(C)OC(=O)N1CCC(CO)C1 ; Cc1ccc(S(=O)(=O)OCC2CCN(C(=O)OC(C)(C)C)C2)cc1 ; CC(C)(C)OC(=O)N1CCC(COc2ccccc2C(F)(F)F)C1 ; FC(F)(F)c1ccccc1OCC1CCNC1 ; COC(=O)c1cc(C)nc(N2CCC(COc3ccccc3C(F)(F)F)C2)n1 ; Cc1cc(C(=O)O)nc(N2CCC(COc3ccccc3C(F)(F)F)C2)n1]

### Example 5: (±)-6-Methyl-2-(3-((2-(trifluoromethyl)phenoxy)methyl)pyrrolidin-1-yl)pyrimidine-4-carboxylic Acid (±)-44

Step A: To a 0° C. solution of (±)-tert-butyl 3-(hydroxymethyl)pyrrolidine-1-carboxylate (±)-39 (2.0 g, 9.93 mmol), Et₃N (2.8 ml, 83.7 mmol), and DMAP (60.4 mg, 0.494 mmol) in CH₂Cl₂ (50 mL) was added TsCl (2.27 g, 11.9 mmol). The resulting mixture was stirred for 16 h while gradually warming to rt under an atmosphere of N₂. The mixture was diluted with saturated aqueous NaOH solution (50 mL) and then extracted with EtOAc (3×100 mL). The combined organic extracts were washed with H₂O (50 mL), brine (50 mL), dried over Na₂SO₄, filtered, and concentrated under reduced pressure. The resulting residue was chromatographed over silica gel (0-30% EtOAc in hexanes) to give (±)-tert-butyl 3-((tosyloxy)methyl)pyrrolidine-1-carboxylate (±)-40 as a white solid (3.1 g, 88%): ¹H NMR (400 MHz, DMSO-d₆) δ 7.76 (d, J=8.4 Hz, 2H), 7.46 (d, J=8.4 Hz, 2H), 3.97 (d, J=6.8 Hz, 2H), 3.28-3.25 (m, 1H), 3.24-3.19 (m, 2H), 3.13-3.10 (m, 1H), 2.85-2.81 (m, 1H), 2.39 (s, 3H), 1.82-1.81 (m, 1H), 1.49-1.47 (m, 1H), 1.33 (s, 9H); ESI MS m/z 356 [M+H]⁺.

Step B: To a solution of (±)-tert-butyl 3-((tosyloxy)methyl)pyrrolidine-1-carboxylate (±)-40 (1.0 g, 2.81 mmol) in DMF (10 mL) were added Cs₂CO₃ (2.74 g, 8.41 mmol) and 2-(trifluoromethyl)phenol (0.410 g, 2.53 mmol) and the resulting mixture was stirred at 80° C. for 16 h under an atmosphere of N₂. The mixture was allowed to cool to rt and then diluted with H₂O (30 mL). The aqueous mixture was extracted with EtOAc (3×50 mL) and the combined organic extracts were washed with H₂O (3×50 mL), brine (50 mL), dried over Na₂SO₄, filtered, and concentrated under reduced pressure. The resulting residue was chromatographed over silica gel (0% to 30% EtOAc in hexanes) to give (±)-tert-butyl 3-((2-(trifluoromethyl)phenoxy)methyl)pyrrolidine-1-carboxylate (±)-41 as a white solid (0.816 g, 84%): ¹H NMR (400 MHz, DMSO-d₆) δ 7.61-7.56 (m, 2H), 7.22 (d, J=8.8 Hz, 1H), 7.06 (t, J=7.6 Hz, 1H), 4.09-4.02 (m, 2H), 3.44-3.35 (m, 1H), 3.36-3.33 (m, 1H), 3.25-3.19 (m, 1H), 3.11-3.01 (m, 1H), 2.62-2.55 (m, 1H), 1.97 (brs, 1H), 1.96-1.64 (m, 1H), 1.35 (s, 9H); ESI MS m/z 346 [M+H]⁺.

Step C: To a 0° C. solution of (±)-tert-butyl 3-((2-(trifluoromethyl)phenoxy)methyl)pyrrolidine-1-carboxylate (±)-41 (0.800 g, 2.32 mmol) in CH₂Cl₂ (10 mL) was added TFA (3.5 mL, 46.3 mmol) and the resulting mixture was stirred for 16 h while gradually warming to rt under an atmosphere of N₂. The mixture was neutralized by carefully pouring it into a solution of saturated aqueous NaHCO₃ (50 mL) and the resulting biphasic mixture was separated. The aqueous layer was further extracted with CH₂Cl₂ (3×50 mL) and the combined organic extracts were washed with brine (50 mL), dried over Na₂SO₄, filtered, and concentrated under reduced pressure to give (±)-3-((2-(trifluoromethyl)phenoxy)methyl)pyrrolidine (±)-42 as a white solid (0.520 g, 90%): ¹H NMR (400 MHz, DMSO-d₆) δ 9.25 (brs, 1H), 7.62-7.57 (m, 2H), 7.22 (d, J=8.4 Hz, 1H), 7.07 (t, J=7.6 Hz, 1H), 4.16-4.05 (m, 2H), 3.38-3.33 (m, 1H), 3.28-3.16 (m, 2H), 2.98 (t, J=8.0 Hz, 1H), 2.77-2.69 (m, 1H), 2.11-2.03 (m, 1H), 1.78-1.69 (m, 1H); ESI MS m/z 346 [M+H]l; ESI MS m/z 246 [M+H]⁺.

Step D: To a solution of (±)-tert-butyl 3-((2-(trifluoromethyl)phenoxy)methyl)pyrrolidine-1-carboxylate (±)-42 (0.265 g 1.08 mmol) in THF (5 mL) were added i-Pr₂NEt (0.6 mL, 3.24 mmol) and methyl 2-chloro-6-methylpyrimidine-4-carboxylate (0.242 g, 1.29 mmol) and the resulting mixture stirred at reflux for 16 h. The mixture was allowed to cool to rt and then concentrated under reduced pressure. The resulting residue was chromatographed over silica gel (0% to 50% EtOAc in hexanes) to give (±)-methyl 6-methyl-2-(3-((2-(trifluoromethyl)phenoxy)methyl)pyrrolidin-1-yl)pyrimidine-4-carboxylate (±)-43 as a white solid (0.362 g, 85%): ¹H NMR (400 MHz, CDCl₃) δ 7.54 (d, J=7.6 Hz, 1H), 7.44 (t, J=7.6 Hz, 1H), 6.99 (s, 1H), 6.97-6.92 (m, 2H), 4.10-4.06 (m, 1H), 4.00-3.96 (m, 1H), 3.91 (s, 3H), 3.89-3.86 (m, 1H), 3.79-3.76 (m, 1H), 3.66-3.61 (m, 1H), 3.51-3.47 (m, 1H), 2.86-2.82 (m, 1H), 2.39 (s, 3H), 2.24-2.20 (m, 1H), 1.99-1.94 (m, 1H); ESI MS m/z 396 [M+H]⁺.

Step E: To a solution of (±)-methyl 6-methyl-2-(3-((2-(trifluoromethyl)phenoxy)methyl)pyrrolidin-1-yl)pyrimidine-4-carboxylate (±)-43 (0.250 g, 0.632 mmol) in CH₃OH (4 mL), THF (4 mL), and H₂O (2 mL) was added LiOH (0.151 g, 6.32 mmol) and the mixture stirred at rt for 16 h. The mixture was concentrated under reduced pressure to remove the volatile solvents and the resulting aqueous mixture was diluted with additional H₂O (10 mL) and acidified with 2 N aqueous HCl to pH=3. The acidified mixture was extracted with EtOAc (3×50 mL) and the combined organic extracts were washed with brine (50 mL), dried over Na₂SO₄, filtered, and concentrated under reduced pressure to give (±)-1-(4-(2-(trifluoromethyl)phenyl)piperidine-1-carbonyl)pyrrolidine-2-carboxylic acid (±)-44 as a white solid (0.160 g, 66%): ¹H NMR (400 MHz, DMSO-d₆) δ 7.59 (m, 2H), 7.24 (m, 1H), 7.06-7.02 (m, 1H), 6.93 (s, 1H), 4.16-4.11 (m, 2H), 3.95-3.61 (m, 2H), 3.50-3.44 (m, 1H), 3.35-3.09 (m, 1H), 2.75-2.68 (m, 1H), 2.35 (s, 3H), 2.14-2.06 (m, 1H), 1.87-1.78 (m, 1H); ESI MS m/z 382 [M+H]⁺; HPLC 98.7% (AUC), t_{R}=14.5 min.

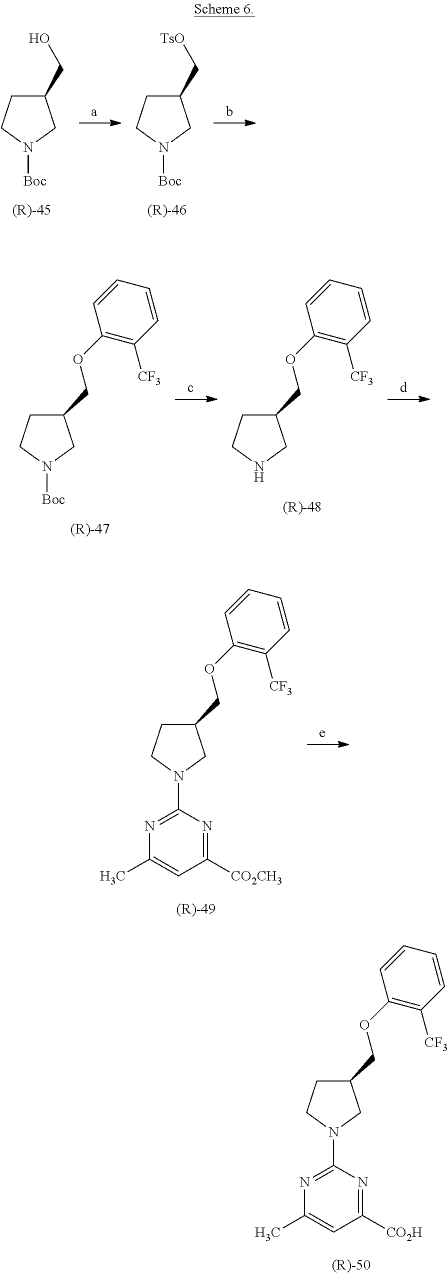 [SMILES: CC(C)(C)OC(=O)N1CC[C@@H](CO)C1 ; Cc1ccc(S(=O)(=O)OC[C@@H]2CCN(C(=O)OC(C)(C)C)C2)cc1 ; CC(C)(C)OC(=O)N1CC[C@@H](COc2ccccc2C(F)(F)F)C1 ; FC(F)(F)c1ccccc1OC[C@@H]1CCNC1 ; COC(=O)c1cc(C)nc(N2CC[C@@H](COc3ccccc3C(F)(F)F)C2)n1 ; Cc1cc(C(=O)O)nc(N2CC[C@@H](COc3ccccc3C(F)(F)F)C2)n1]

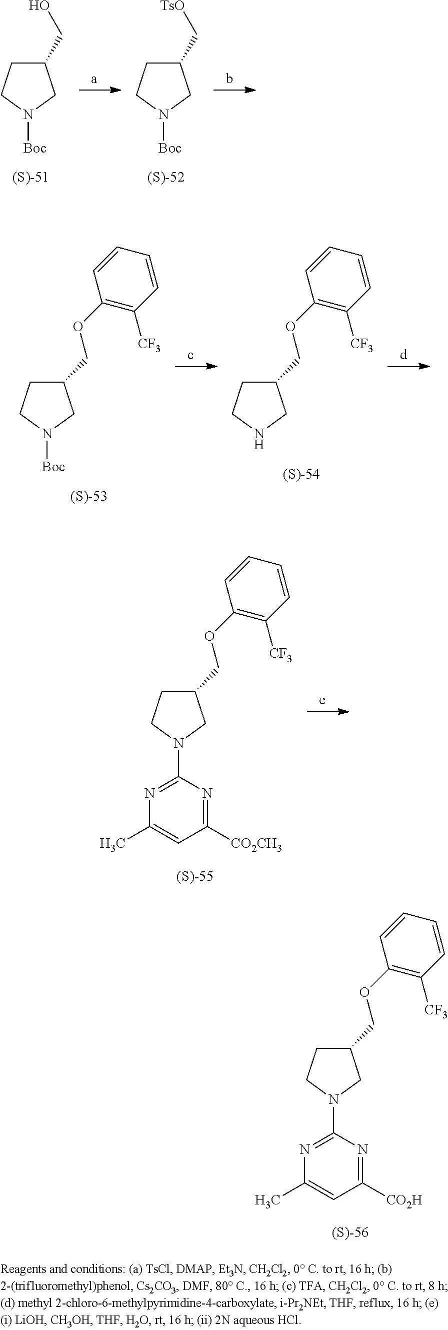 [SMILES: CC(C)(C)OC(=O)N1CC[C@H](CO)C1 ; Cc1ccc(S(=O)(=O)OC[C@H]2CCN(C(=O)OC(C)(C)C)C2)cc1 ; CC(C)(C)OC(=O)N1CC[C@H](COc2ccccc2C(F)(F)F)C1 ; FC(F)(F)c1ccccc1OC[C@H]1CCNC1 ; COC(=O)c1cc(C)nc(N2CC[C@H](COc3ccccc3C(F)(F)F)C2)n1 ; Cc1cc(C(=O)O)nc(N2CC[C@H](COc3ccccc3C(F)(F)F)C2)n1]

### Example 6

(R)-6-Methyl-2-(3-((2-(trifluoromethyl)phenoxy)methyl)pyrrolidin-1-yl)pyrimidine-4-carboxylic Acid (R)-50. Compound (R)-50 was prepared from tert-butyl (R)-3-(hydroxymethyl)pyrrolidine-1-carboxylate (R)-45 according to a similar procedure described for the synthesis of (±)-44: ¹H NMR (400 MHz, CDCl₃) δ 7.55 (d, J=7.6 Hz, 1H), 7.45 (t, J=7.6 Hz, 1H), 7.12 (s, 1H), 7.01-6.94 (m, 2H), 4.07-4.06 (m, 2H), 3.86-3.77 (m, 2H), 3.62-3.51 (m, 2H), 2.89-2.86 (m, 1H), 2.43 (s, 3H), 2.27-2.23 (m, 1H), 2.14-2.01 (m, 1H); ESI MS m/z 382 [M+H]⁺; HPLC>99% (AUC), t_{R}=14.5 min.

### Example 7: (S)-6-Methyl-2-(3-((2-(trifluoromethyl)phenoxy)methyl)pyrrolidin-1-yl)pyrimidine-4-carboxylic Acid (S)-56

Compound (S)-56 was prepared from tert-butyl (S)-3-(hydroxymethyl)pyrrolidine-1-carboxylate (S)-51 according to a similar procedure described for the synthesis of (±)-44: ¹H NMR (400 MHz, CDCl₃) δ 7.56 (d, J=8.0 Hz, 1H), 7.46 (t, J=8.4 Hz, 1H), 7.12 (s, 1H), 7.01-6.94 (m, 2H), 4.07-4.06 (m, 2H), 3.86-3.77 (m, 2H), 3.62-3.51 (m, 2H), 2.90-2.85 (m, 1H), 2.44 (s, 3H), 2.26-2.24 (m, 1H), 2.14-2.01 (m, 1H); ESI MS m/z 382 [M+H]⁺; HPLC>99% (AUC), t_{R}=14.5 min.

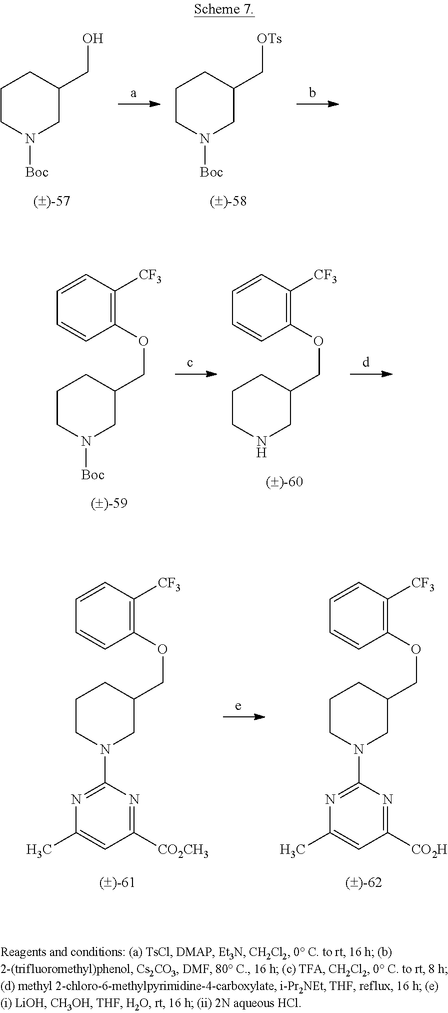 [SMILES: CC(C)(C)OC(=O)N1CCCC(CO)C1 ; Cc1ccc(S(=O)(=O)OCC2CCCN(C(=O)OC(C)(C)C)C2)cc1 ; CC(C)(C)OC(=O)N1CCCC(COc2ccccc2C(F)(F)F)C1 ; FC(F)(F)c1ccccc1OCC1CCCNC1 ; COC(=O)c1cc(C)nc(N2CCCC(COc3ccccc3C(F)(F)F)C2)n1 ; Cc1cc(C(=O)O)nc(N2CCCC(COc3ccccc3C(F)(F)F)C2)n1]

### Example 8: (±)-6-Methyl-2-(3-((2-(trifluoromethyl)phenoxy) methyl)piperidin-1-yl)pyrimidine-4-carboxylic Acid (±)-62

Step A: To a 0° C. solution of (±)-tert-butyl 3-(hydroxymethyl)piperidine-1-carboxylate (±)-57 (1.0 g, 4.65 mmol) in CH₂Cl₂ (20 mL) were added Et₃N (0.81 ml, 5.80 mmol) and DMAP (52.0 mg, 0.426 mmol) followed by the addition of TsCl (0.883 g, 4.65 mmol). The resulting solution was stirred for 16 h while gradually warming to rt. The reaction mixture was diluted with saturated aqueous NaOH solution (50 mL) and was extracted with EtOAc (3×100 mL). The combined organic extracts were washed with H₂O (100 mL) and brine (100 mL), dried over Na₂SO₄, filtered, and concentrated under reduced pressure to give (±)-tert-butyl 3-((tosyloxy)methyl)piperidine-1-carboxylate (±)-58 as a colorless liquid (1.6 g, 94%): ESI MS m/z 370 [M+H]⁺.

Step B: To a solution of (±)-tert-butyl 3-((tosyloxy)methyl)piperidine-1-carboxylate (±)-58 (0.500 g 1.35 mmol) in DMF (20 mL) were added Cs₂CO₃ (0.650 g, 2.00 mmol) and 2-(trifluoromethyl)phenol (0.219 g, 1.35 mmol) and the resulting mixture was stirred at 80° C. for 16 h under an atmosphere of N₂. The reaction mixture was allowed to cool to rt and then diluted with H₂O (50 mL) and extracted with EtOAc (3×50 mL). The combined organic extracts were washed with H₂O (3×50 mL), brine (50 mL), dried over Na₂SO₄, filtered, and concentrated under reduced pressure to give (±)-tert-butyl 3-((2-(trifluoromethyl)phenoxy)methyl)piperidine-1-carboxylate (±)-59 as a brown oil (0.400 g, 82%): ESI MS m/z 360 [M+H]⁺.

Step C: To a 0° C. solution of (±)-tert-butyl 3-((2-(trifluoromethyl)phenoxy)methyl)piperidine-1-carboxylate (±)-59 (0.400 g, 1.11 mmol) in CH₂Cl₂ (20 mL) was added TFA (0.85 mL, 11.1 mmol) and the resulting solution was stirred for 8 h while gradually warming to rt. The mixture was neutralized by carefully pouring it into a solution of saturated aqueous NaHCO₃ (10 mL). The biphasic mixture was separated and the aqueous layer was further extracted with CH₂Cl₂ (3×20 mL). The combined organic extracts were washed with brine (20 mL), dried over Na₂SO₄, filtered, and concentrated under reduced pressure to give (±)-3-((2-(trifluoromethyl)phenoxy)methyl)piperidine (±)-60 as a white solid (0.250 g, 86%): ESI MS m/z 260 [M+H]⁺.

Step D: To a solution of (i)-3-((2-(trifluoromethyl)phenoxy)methyl)piperidine (±)-60 (0.100 g, 0.385 mmol) in THF (5 mL) were added i-Pr₂NEt (0.20 mL, 1.16 mmol) and methyl 2-chloro-6-methylpyrimidine-4-carboxylate (71.8 mg, 0.385 mmol) and the resulting mixture stirred at reflux for 16 h under an atmosphere of N₂. The mixture was allowed to cool to rt and then concentrated under reduced pressure. The resulting residue was chromatographed over silica gel (0% to 50% EtOAc in hexanes) to give (±)-methyl 6-methyl-2-(3-((2-(trifluoromethyl)phenoxy)methyl)piperidin-1-yl)pyrimidine-4-carboxylate (±)-61 as a white solid (0.137 g, 87%): ¹H NMR (400 MHz, DMSO-d₆) δ 7.59-7.55 (m, 2H), 7.22 (d, J=8.4 Hz, 1H), 7.05 (t, J=7.6 Hz, 1H), 6.94 (s, 1H), 4.79 (d, J=12.8 Hz, 1H), 4.55 (d, J=12.8 Hz, 1H), 4.04-3.96 (m, 2H), 3.80 (s, 3H), 2.93-2.83 (m, 3H), 2.29 (s, 3H), 1.85-1.71 (m, 4H); ESI MS m/z 410 [M+H]⁺.

Step E: To a solution of (±)-methyl 6-methyl-2-(3-((2-(trifluoromethyl)phenoxy)methyl)piperidin-1-yl)pyrimidine-4-carboxylate (±)-61 (0.100 g, 0.244 mmol) in CH₃OH (4 mL), THF (4 mL), and H₂O (2 mL) was added LiOH (58.4 mg, 2.44 mmol) and the mixture stirred at rt for 16 h. The mixture was concentrated under reduced pressure to remove the volatile solvents and the resulting aqueous mixture was diluted with additional H₂O (10 mL) and acidified with 2 N aqueous HCl to pH=3. The acidified mixture was extracted with EtOAc (3×50 mL) and the combined organic extracts were washed with brine (50 mL), dried over Na₂SO₄, filtered, and concentrated under reduced pressure to give (±)-6-methyl-2-(3-((2-(trifluoromethyl)phenoxy)methyl)piperidin-1-yl)pyrimidine-4-carboxylic acid (±)-62 as a white solid (96.4 mg, 66%): ¹H NMR (400 MHz, DMSO-d₆) δ 7.60-7.56 (m, 2H), 7.23 (d, J=8.8 Hz, 1H), 7.06 (t, J=8.0 Hz, 1H), 6.91 (s, 1H), 4.77 (d, J=10.4 Hz, 1H), 4.57 (d, J=13.2 Hz, 1H), 4.02-4.00 (m, 2H), 2.92-2.84 (m, 3H), 2.29 (s, 3H), 1.93-1.71 (m, 4H), 1.41-1.39 (m, 2H); ESI MS m/z 396 [M+H]; HPLC>99% (AUC), t_{R}=16.2 min.

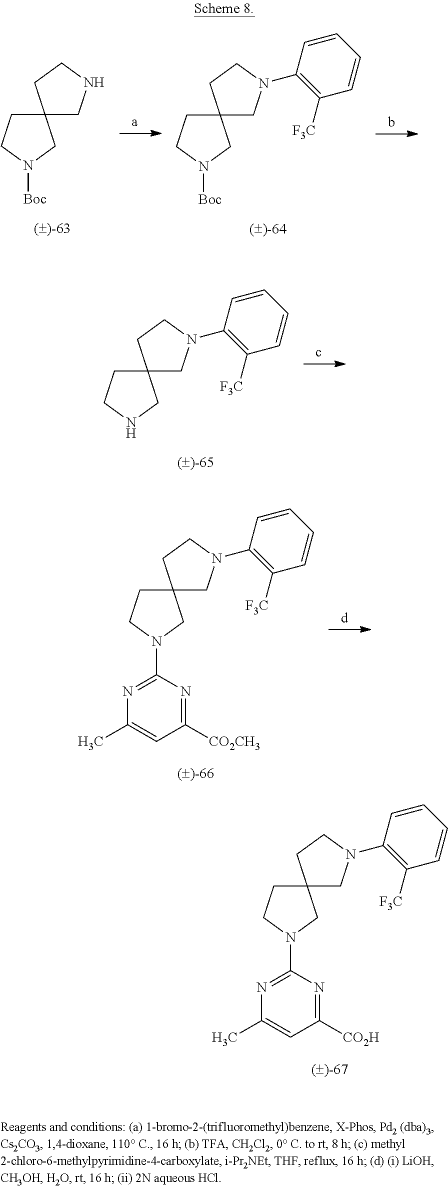 [SMILES: CC(C)(C)OC(=O)N1CCC2(CCNC2)C1 ; CC(C)(C)OC(=O)N1CCC2(CCN(c3ccccc3C(F)(F)F)C2)C1 ; FC(F)(F)c1ccccc1N1CCC2(CCNC2)C1 ; COC(=O)c1cc(C)nc(N2CCC3(CCN(c4ccccc4C(F)(F)F)C3)C2)n1 ; Cc1cc(C(=O)O)nc(N2CCC3(CCN(c4ccccc4C(F)(F)F)C3)C2)n1]

### Example 9: (±)-6-Methyl-2-(7-(2-(trifluoromethyl)phenyl)-2,7-diazaspiro[4.4]nonan-2-yl)pyrimidine-4-carboxylic Acid (±)-67

Step A: A mixture of (±)-tert-butyl 2,7-diazaspiro[4.4]nonane-2-carboxylate (±)-63 (0.250 g, 1.11 mmol) and 1-bromo-2-(trifluoromethyl)benzene (0.273 g, 1.22 mmol) in 1,4-dioxane was degassed with N2 for 5 min followed by the addition of Cs₂CO₃ (1.08 g, 3.31 mmol), X-Phos (0.105 mg, 0.223 mmol) and Pd₂(dba)₃ (0.101 g, 0.112 mmol). The reaction mixture was stirred at 110° C. for 16 h in a sealed vessel. The mixture was then allowed to cool to rt and concentrated under reduced pressure. The resulting residue was chromatographed over silica gel (0% to 30% EtOAc in hexane) to give (±)-methyl tert-butyl 7-(2-(trifluoromethyl)phenyl)-2,7-diazaspiro[4.4]nonane-2-carboxylate (±)-64 as an off-white amorphous solid (0.230 g, 56%): ESI MS m/z 371 [M+H]⁺.

Step B: To a 0° C. solution of (±)-methyl tert-butyl 7-(2-(trifluoromethyl)phenyl)-2,7-diazaspiro[4.4]nonane-2-carboxylate (±)-64 (0.100 g, 2.69 mmol) in CH₂Cl₂ (5 mL) was added TFA (0.20 mL, 2.61 mmol) and the resulting solution was stirred for 8 h while gradually warming to rt. The mixture was neutralized by carefully pouring it into a solution of saturated aqueous NaHCO₃ (10 mL). The biphasic mixture was separated and the aqueous layer was further extracted with CH₂Cl₂ (3×20 mL). The combined organic extracts were washed with brine (20 mL), dried over Na₂SO₄, filtered, and concentrated under reduced pressure to give (±)-2-(2-(trifluoromethyl)phenyl)-2,7-diazaspiro[4.4]nonane (±)-65 as a white solid (65.0 mg, 90%): ESI MS m/z 271 [M+H]⁺.

Step C: To a solution of (±)-2-(2-(trifluoromethyl)phenyl)-2,7-diazaspiro[4.4]nonane (±)-65 (0.150 g, 0.554 mmol) in THF (5 mL) were added i-Pr₂NEt (0.29 mL, 1.66 mmol) and methyl 2-chloro-6-methylpyrimidine-4-carboxylate (0.103 g, 0.554 mmol) and the resulting mixture stirred at reflux for 16 h under an atmosphere of N₂. The mixture was allowed to cool to rt and then concentrated under reduced pressure. The resulting residue was chromatographed over silica gel (0% to 50% EtOAc in hexanes) to give (±)-methyl 6-methyl-2-(7-(2-(trifluoromethyl)phenyl)-2,7-diazaspiro[4.4]nonan-2-yl)pyrimidine-4-carboxylate (±)-66 as a white solid (0.205 g, 88%): ¹H NMR (400 MHz, CDCl₃) δ 7.55 (d, J=6.4 Hz, 1H), 7.35 (t, J=7.6 Hz, 1H), 7.00 (s, 1H), 6.95 (d, J=8.4 Hz, 1H), 6.87 (t, J=7.6 Hz, 1H), 3.91 (s, 3H), 3.74-3.61 (m, 4H), 3.47-3.44 (m, 2H), 3.30 (s, 2H), 2.39 (s, 3H), 2.08-1.93 (m, 4H); ESI MS m/z 421 [M+H]i.

Step D: To a solution of (±)-methyl 6-methyl-2-(7-(2-(trifluoromethyl)phenyl)-2,7-diazaspiro[4.4]nonan-2-yl)pyrimidine-4-carboxylate (±)-66 (0.100 g, 0.237 mmol) in CH₃OH (4 mL), THF (4 mL), and H₂O (2 mL) was added LiOH (56.9 mg, 2.37 mmol) and the mixture stirred at rt for 16 h. The mixture was concentrated under reduced pressure to remove the volatile solvents and the resulting aqueous mixture was diluted with additional H₂O (10 mL) and acidified with 2 N aqueous HCl to pH=3. The acidified mixture was extracted with EtOAc (3×50 mL) and the combined organic extracts were washed with brine (50 mL), dried over Na₂SO₄, filtered, and concentrated under reduced pressure to give (±)-6-methyl-2-(7-(2-(trifluoromethyl)phenyl)-2,7-diazaspiro[4.4]nonan-2-yl)pyrimidine-4-carboxylic acid (±)-67 as a white solid (65.5 mg, 68%): ¹H NMR (400 MHz, CDCl₃) δ 7.56 (d, J=8.0 Hz, 1H), 7.37 (t, J=7.6 Hz, 1H), 7.13 (s, 1H), 7.01-6.99 (m, 1H), 6.93-6.88 (m, 1H), 3.67-3.55 (m, 4H), 3.46 (t, J=6.8 Hz, 2H), 3.31-3.26 (m, 2H), 2.44 (s, 3H), 2.10-1.97 (m, 4H); ESI MS m/z 407 [M+H]⁺; HPLC 98.4% (AUC), t_{R}=15.5 min.

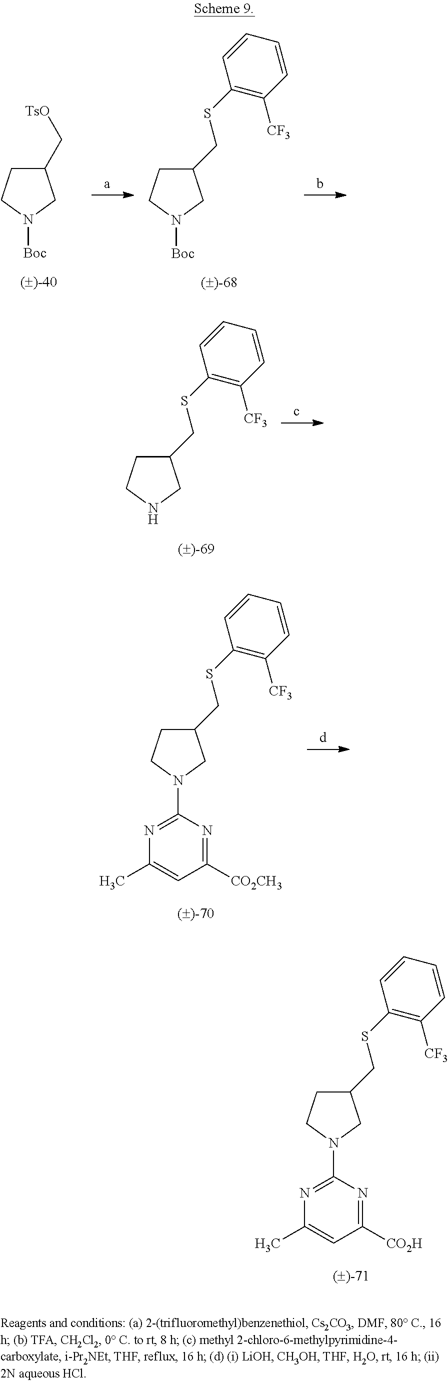 [SMILES: Cc1ccc(S(=O)(=O)OCC2CCN(C(=O)OC(C)(C)C)C2)cc1 ; CC(C)(C)OC(=O)N1CCC(CSc2ccccc2C(F)(F)F)C1 ; FC(F)(F)c1ccccc1SCC1CCNC1 ; COC(=O)c1cc(C)nc(N2CCC(CSc3ccccc3C(F)(F)F)C2)n1 ; Cc1cc(C(=O)O)nc(N2CCC(CSc3ccccc3C(F)(F)F)C2)n1]

### Example 10: (±)-6-Methyl-2-(3-(((2-(trifluoromethyl)phenyl)thio)methyl)pyrrolidin-1-yl)pyrimidine-4-carboxylic Acid (±)-71

Compound (±)-71 was prepared from (±)-tert-butyl 3-((tosyloxy)methyl)pyrrolidine-1-carboxylate (±)-40 and 2-(trifluoromethyl)benzenethiol according to a similar procedure described for the synthesis of (±)-44: ¹H NMR (400 MHz, acetone-d₆) δ 7.72 (t, J=7.2 Hz, 2H), 7.61 (t, J=7.2 Hz, 1H), 7.40 (t, J=8.0 Hz, 1H), 7.05 (s, 1H), 3.89-3.70 (m, 2H), 3.59-3.55 (m, 1H), 3.42-3.74 (m, 1H), 3.25 (d, J=7.2 Hz, 2H), 2.64-2.62 (m, 1H), 2.40 (s, 3H), 2.27-2.24 (m, 1H), 1.92-1.87 (m, 1H); ESI MS m/z 398 [M+H]⁺; HPLC 97.1% (AUC), t_{R}=14.9 min.

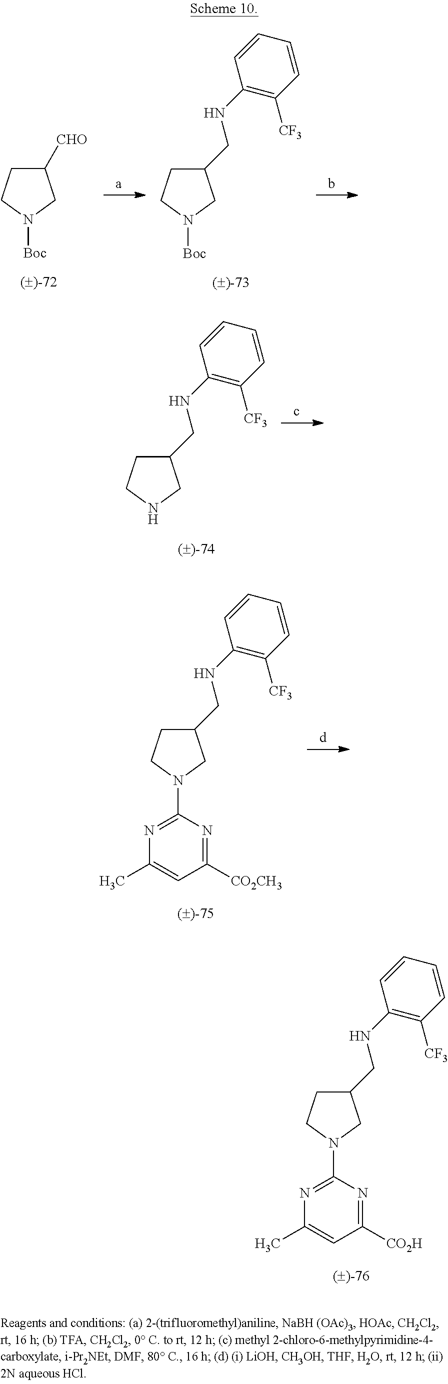 [SMILES: CC(C)(C)OC(=O)N1CCC(C=O)C1 ; CC(C)(C)OC(=O)N1CCC(CNc2ccccc2C(F)(F)F)C1 ; FC(F)(F)c1ccccc1NCC1CCNC1 ; COC(=O)c1cc(C)nc(N2CCC(CNc3ccccc3C(F)(F)F)C2)n1 ; Cc1cc(C(=O)O)nc(N2CCC(CNc3ccccc3C(F)(F)F)C2)n1]

### Example 11: (±)-6-Methyl-2-(3-(((2-(trifluoromethyl)phenyl) amino) methyl)pyrrolidin-1-yl)pyrimidine-4-carboxylic Acid (±)-76

Step A: To a solution of (±)-tert-butyl 3-formylpyrrolidine-1-carboxylate (±)-72 (0.300 g, 1.51 mmol) and 2-(trifluoromethyl)aniline (0.242 g, 1.51 mmol) in CH₂Cl₂ (10 mL) was added NaBH(OAc)₃ (0.960 g, 4.53 mmol) and the mixture stirred at rt for 16 h. The mixture was washed with saturated aqueous NaHCO₃ solution (5 mL), brine (5 mL), dried over Na₂SO₄, filtered, and concentrated under reduced pressure to give crude (±)-tert-butyl 3-(((2-(trifluoromethyl)phenyl)amino)methyl)pyrrolidine-1-carboxylate (±)-73 as an oil, which was used as is in the next step (0.400 g, 77% crude yield): ESI MS m/z 345 [M+H]⁺.

Step B: Step B: To a 0° C. solution of (±)-tert-butyl 3-(((2-(trifluoromethyl)phenyl) amino)methyl)pyrrolidine-1-carboxylate (±)-73 (0.400 g, 1.16 mmol) in CH₂Cl₂ (5 mL) was added TFA (0.89 mL, 11.6 mmol) and the resulting solution was stirred for 12 h while gradually warming to rt. The mixture was neutralized by carefully pouring it into a solution of saturated aqueous NaHCO₃ (10 mL). The biphasic mixture was separated and the aqueous layer was further extracted with CH₂Cl₂ (3×20 mL). The combined organic extracts were washed with brine (20 mL), dried over Na₂SO₄, filtered, and concentrated under reduced pressure to give (±)-N-(pyrrolidin-3-ylmethyl)-2-(trifluoromethyl)aniline (±)-74 as a yellow oil and used as is in the next step (0.360 g): ESI MS m/z 245 [M+H]⁺.

Step C: To a solution of ((±)-N-(pyrrolidin-3-ylmethyl)-2-(trifluoromethyl)aniline (±)-74 (0.360 g, 1.16 mmol) in THF (5 mL) were added i-Pr₂NEt (0.61 mL, 3.48 mmol) and methyl 2-chloro-6-methylpyrimidine-4-carboxylate (0.216 g, 1.16 mmol) and the resulting mixture stirred at reflux for 16 h under an atmosphere of N₂. The mixture was allowed to cool to rt and then concentrated under reduced pressure. The resulting residue was chromatographed over silica gel (0% to 50% EtOAc in hexanes) to give (±)-methyl 6-methyl-2-(3-(((2-(trifluoromethyl)phenyl)amino)methyl)pyrrolidin-1-yl)pyrimidine-4-carboxylate (±)-75 as a white solid (0.290 g, 63%): ¹H NMR (400 MHz, CDCl₃) δ 7.43-7.40 (m, 1H), 7.35-7.31 (m, 1H), 7.00 (s, 1H), 6.71-6.68 (m, 2H), 4.40 (brs, 1H), 3.91 (s, 3H), 3.90-3.86 (m, 1H), 3.79-3.58 (m, 1H), 3.41-3.19 (m, 4H), 2.68-2.62 (m, 1H), 2.40 (s, 3H), 2.22-2.16 (m, 1H), 1.82-1.77 (m, 1H); ESI MS m/z 395 [M+H]⁺.

Step D: To a solution of (±)-methyl 6-methyl-2-(3-(((2-(trifluoromethyl)phenyl)amino)methyl)pyrrolidin-1-yl)pyrimidine-4-carboxylate ((±)-75, 0.110 g, 0.278 mmol) in CH₃OH (4 mL), THF (4 mL), and H₂O (2 mL) was added LiOH (56.9 mg, 2.37 mmol) and the mixture stirred at rt for 16 h. The mixture was concentrated under reduced pressure to remove the volatile solvents and the resulting aqueous mixture was diluted with additional H₂O (10 mL) and acidified with 2 N aqueous HCl to pH=3. The acidified mixture was extracted with EtOAc (3×50 mL) and the combined organic extracts were washed with brine (50 mL), dried over Na₂SO₄, filtered, and concentrated under reduced pressure to give (±)-6-methyl-2-(3-(((2-(trifluoromethyl)phenyl)amino)methyl)pyrrolidin-1-yl)pyrimidine-4-carboxylic acid (±)-76 as a white solid (90.0 mg, 68%): ¹H NMR (400 MHz, DMSO-d₆) δ 7.36-7.32 (m, 2H), 6.84-6.79 (m, 2H), 6.20 (t, J=7.6 Hz, 1H), 5.53 (brs, 1H), 3.63-3.58 (m, 2H), 3.41-3.39 (m, 1H), 3.25-3.17 (m, 3H), 2.58-2.57 (m, 1H), 2.23 (s, 3H), 1.98-1.95 (m, 1H), 1.70-1.67 (m, 1H); ESI MS m/z 381 [M+H]⁺; HPLC>99% (AUC), t_{R}=14.5 min.

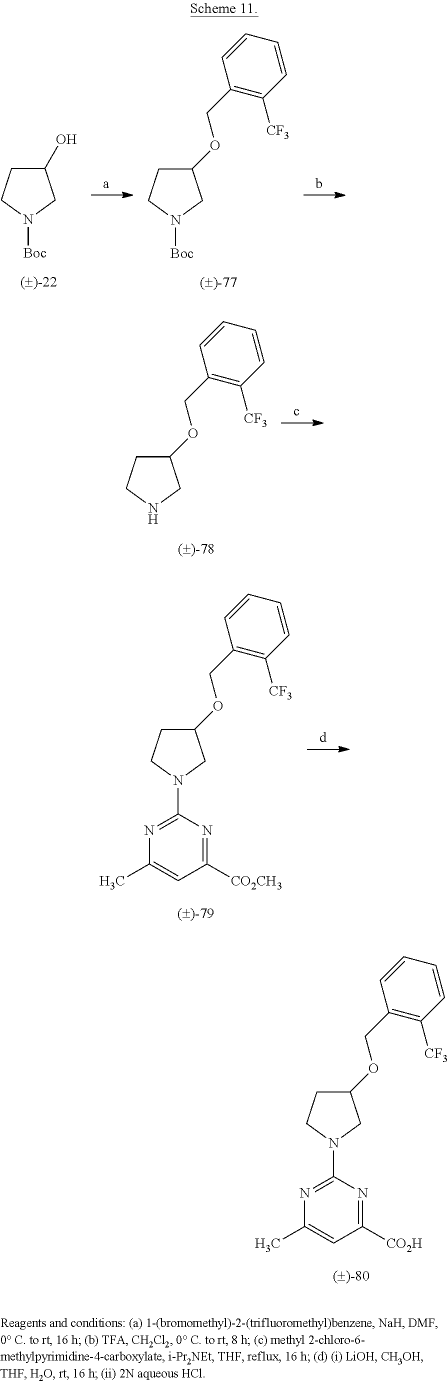 [SMILES: CC(C)(C)OC(=O)N1CCC(O)C1 ; CC(C)(C)OC(=O)N1CCC(OCc2ccccc2C(F)(F)F)C1 ; FC(F)(F)c1ccccc1COC1CCNC1 ; COC(=O)c1cc(C)nc(N2CCC(OCc3ccccc3C(F)(F)F)C2)n1 ; Cc1cc(C(=O)O)nc(N2CCC(OCc3ccccc3C(F)(F)F)C2)n1]

### Example 12: (±)-6-Methyl-2-(3-((2-(trifluoromethyl)benzyl)oxy)pyrrolidin-1-yl)pyrimidine-4-carboxylic Acid (±)-80

Step A: To a 0° C. cooled solution of tert-butyl 3-hydroxypyrrolidine-1-carboxylate (±)-22 (0.500 g, 2.67 mmol) in DMF (5 mL) was added NaH (0.267 g, 6.68 mmol). The mixture stirred at 0° C. under an atmosphere of N2 for 30 min, then 1-(bromomethyl)-2-(trifluoromethyl)benzene (0.766 g, 3.20 mmol) was added and the resulting mixture stirred for 16 h while gradually warming to rt. The mixture was cooled back to 0° C. and carefully quenched via dilution with H₂O (20 mL). The aqueous mixture was extracted with EtOAc (3×30 mL) and the combined organic extracts were washed with H₂O (3×30 mL) and brine (30 mL), dried over Na₂SO₄, filtered, and concentrated under reduced pressure to give (±)-tert-butyl 3-((2-(trifluoromethyl)benzyl)oxy)pyrrolidine-1-carboxylate (±)-77 as an off-white solid (0.900 g, 97% crude yield), which was used as is in the next step: ESI MS m/z 346 [M+H]⁺.

Step B: To a 0° C. cooled solution of (±)-tert-butyl 3-((2-(trifluoromethyl)benzyl)oxy)pyrrolidine-1-carboxylate (±)-77 (0.900 g 2.61 mmol) in CH₂Cl₂ (5 mL) was added TFA (2.0 mL, 26.0 mmol) and the resulting solution stirred for 8 h. while gradually warming to rt. The mixture was neutralized by carefully pouring it into aqueous saturated NaHCO₃ solution (30 mL). The biphasic mixture was separated and the aqueous layer was further extracted with CH₂Cl₂ (3×30 mL). The combined organic extracts were washed with brine (30 mL), dried over Na₂SO₄, filtered, and concentrated under reduced pressure to give (±)-3-((2-(trifluoromethyl)benzyl)oxy)pyrrolidine (±)-78 as a white solid (0.450 g, 70% crude yield): ESI MS m/z 246[M+H]⁺.

Step C: To a solution of (i)-3-((2-(trifluoromethyl)benzyl)oxy)pyrrolidine (±)-78 (0.100 g, 0.407 mmol) in THF (5 mL) were added i-Pr₂NEt (0.25 mL, 1.23 mmol) and methyl 2-chloro-6-methylpyrimidine-4-carboxylate (76.2 mg, 0.408 mmol) and the resulting solution was stirred at 80° C. for 16 h under an atmosphere of N₂. The mixture was allowed to cool to rt and then concentrated under reduced pressure. The resulting residue was chromatographed over silica gel (0% to 50% EtOAc in hexanes) to give (±)-methyl 6-methyl-2-(3-((2-(trifluoromethyl)benzyl)oxy)pyrrolidin-1-yl)pyrimidine-4-carboxylate (±)-79 as a white solid (0.110 g, 68%): ¹H NMR (400 MHz, acetone-d₆) δ 7.75-7.68 (m, 2H), 7.62 (t, J=7.2 Hz, 1H), 7.47 (t, J=7.6 Hz, 1H), 6.97 (s, 1H), 4.78-4.76 (m, 2H), 4.41-4.39 (m, 1H), 3.85 (s, 3H), 3.74-3.66 (m, 4H), 2.36 (s, 3H), 2.31-2.04 (m, 2H); ESI MS m/z 396 [M+H]⁺.

Step D: To a solution of (±)-methyl 6-methyl-2-(3-((2-(trifluoromethyl)benzyl)oxy)pyrrolidin-1-yl)pyrimidine-4-carboxylate ((±)-79, 90.0 mg, 0.228 mmol) in CH₃OH (4 mL), THF (4 mL) and H₂O (2 mL) was added LiOH (54.5 mg, 2.28 mmol) and the mixture stirred at rt for 16 h. the mixture was and concentrated under reduced pressure to remove the volatile solvents and the resulting aqueous layer was diluted with additional H₂O (10 mL) and acidified with 2 N aqueous HCl to pH=3. The mixture was extracted with EtOAc (3×30 mL) and the combined organic extracts were washed with brine (30 mL), dried over Na₂SO₄, filtered, and concentrated under reduced pressure to give (±)-6-methyl-2-(3-((2-(trifluoromethyl)benzyl)oxy)pyrrolidin-1-yl)pyrimidine-4-carboxylic acid (±)-80 as a white solid (60.0 mg, 85%): ¹H NMR (400 MHz, DMSO-d₆) δ 7.67-7.59 (m, 3H), 7.47-7.45 (m, 1H), 6.90 (s, 1H), 4.64-4.58 (m, 2H), 4.28-4.23 (m, 1H), 3.66-3.46 (m, 4H), 2.25 (s, 3H), 2.05-1.98 (m, 2H); ESI MS m/z 382 [M+H]⁺; HPLC 98.1% (AUC), t_{R}=14.4 min.

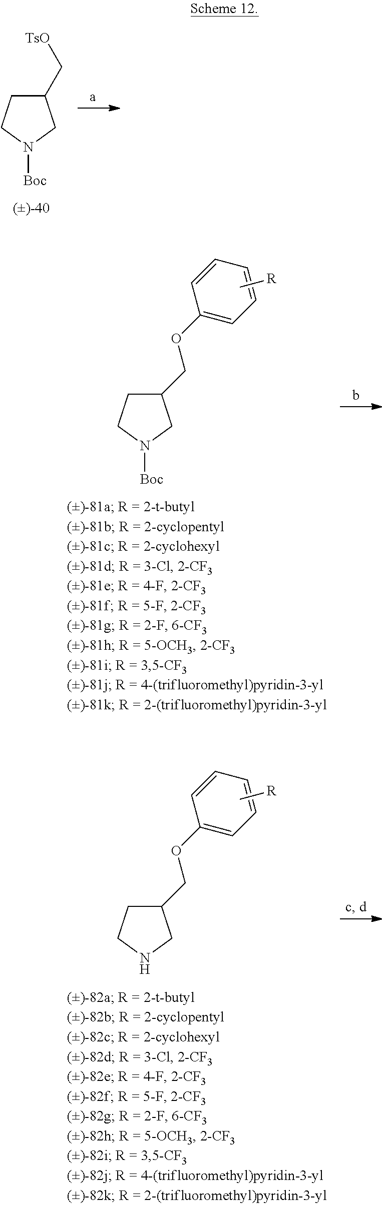

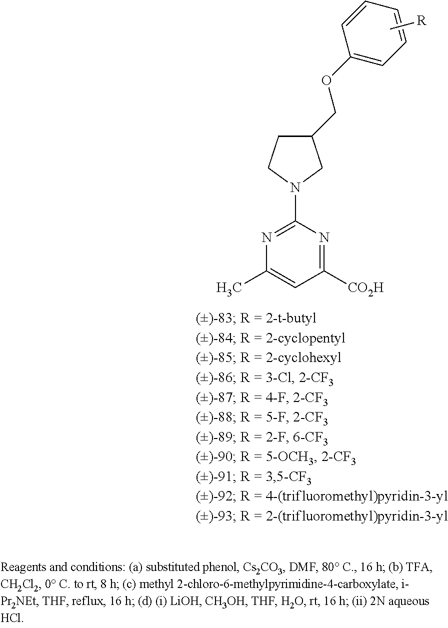

### Example 13: (±)-2-(3-((2-(Tert-butyl)phenoxy)methyl)pyrrolidin-1-yl)-6-methylpyrimidine-4-carboxylic Acid (±)-83

Compound (±)-83 was prepared from 2-(tert-butyl)phenol and (±)-tert-butyl 3-((tosyloxy)methyl)pyrrolidine-1-carboxylate (±)-40 according to a similar procedure described for the synthesis of (±)-44: ¹H NMR (400 MHz, CDCl₃) δ 7.28 (d, J=8.0 Hz, 1H), 7.17-7.12 (m, 2H), 6.91-6.82 (m, 2H), 4.06-4.00 (m, 3H), 3.93-3.82 (s, 1H), 3.63-3.51 (m, 2H), 2.93-2.90 (m, 1H), 2.44 (s, 3H), 2.31-2.28 (m, 1H), 2.00-1.98 (m, 1H), 1.38 (s, 9H); ESI MS m/z 370 [M+H]⁺; HPLC 96.4% (AUC), t_{R}=16.2 min.

### Example 14: (±)-2-(3-((2-Cyclopentylphenoxy)methyl)pyrrolidin-1-yl)-6-methylpyrimidine-4-carboxylic Acid (±)-84

Compound (±)-84 was prepared from 2-cyclopentylphenol and (±)-tert-butyl 3-((tosyloxy)methyl)pyrrolidine-1-carboxylate (±)-40 according to a similar procedure described for the synthesis of (±)-44: ¹H NMR (400 MHz, DMSO-d₆) δ 7.16-7.07 (m, J=8.0 Hz, 2H), 6.94 (s, 1H), 6.90 (d, J=7.6 Hz, 1H), 6.83 (t, J=7.2 Hz, 1H), 3.98 (d, J=6.4 Hz, 2H), 3.76-3.63 (m, 2H), 3.53-3.39 (m, 2H), 3.22-3.15 (m, 1H), 2.77-2.71 (m, 1H), 2.32 (s, 3H), 2.17-2.11 (m, 1H), 1.90-1.83 (m, 3H), 1.68-1.66 (m, 2H), 1.61-1.44 (n, 4H); ESI MS m/z 382 [M+H]⁺; HPLC 95.2% (AUC), t_{R}=16.4 min.

### Example 15: (±)-2-(3-((2-Cyclohexylphenoxy)methyl)pyrrolidin-1-yl)-6-methylpyrimidine-4-carboxylic Acid (±)-85

Compound (±)-85 was prepared from 2-cyclohexylphenol and (±)-tert-butyl 3-((tosyloxy)methyl)pyrrolidine-1-carboxylate (±)-40 according to a similar procedure described for the synthesis of (±)-44: ¹H NMR (400 MHz, DMSO-d₆) ¹H NMR (400 MHz, CDCl₃) δ 7.17-7.15 (m, 1H), 7.13-7.11 (m, 1H), 7.12 (s, 1H), 6.92-6.88 (m, 1H), 6.82 (d, J=8.4 Hz, 1H), 4.01-3.93 (m, 2H), 3.87-3.80 (m, 2H), 3.69-3.48 (m, 2H), 2.89-2.84 (m, 2H), 2.43 (s, 3H), 2.26-2.15 (m, 1H), 1.99-1.96 (m, 1H), 1.83-1.69 (m, 5H), 1.41-1.22 (n, 5H); ESI MS m/z 396 [M+H]⁺; HPLC 98.3% (AUC), t_{R}=17.1 min.

### Example 16: (k)-2-(3-((3-Chloro-2-(trifluoromethyl)phenoxy)methyl)pyrrolidin-1-yl)-6-methylpyrimidine-4-carboxylic Acid (±)-86

Compound (±)-86 was prepared from 3-chloro-2-(trifluoromethyl)phenol and (±)-tert-butyl 3-((tosyloxy)methyl)pyrrolidine-1-carboxylate (±)-40 according to a similar procedure described for the synthesis of (±)-44: ¹H NMR (400 MHz, DMSO-d₆) δ 7.56 (d, J=8.4 Hz, 1H), 7.26 (d, J=8.4 Hz, 1H), 7.19 (d, J=7.6 Hz, 1H), 6.94 (s, 1H), 4.19-4.10 (m, 2H), 3.76-3.63 (m, 2H), 3.51-3.44 (m, 1H), 3.33-3.32 (m, 1H), 2.77-2.70 (m, 1H), 2.32 (s, 3H), 2.15-2.07 (m, 1H), 1.87-1.79 (n, 1H); ESI MS m/z 416 [M+H]⁺; HPLC 99.0% (AUC), t_{R}=15.1 min.

### Example 17: (W)-2-(3-((4-Fluoro-2-(trifluoromethyl)phenoxy)methyl)pyrrolidin-1-yl)-6-methylpyrimidine-4-carboxylic Acid (±)-87

Compound (±)-87 was prepared from 4-fluoro-2-(trifluoromethyl)phenol and (±)-tert-butyl 3-((tosyloxy)methyl)pyrrolidine-1-carboxylate (±)-40 according to a similar procedure described for the synthesis of (±)-44: ¹H NMR (400 MHz, DMSO-d₆) δ 7.48-7.44 (m, 2H), 7.28-7.25 (m, 1H), 6.92 (s, 1H), 4.12-4.08 (m, 2H), 3.72-3.68 (m, 1H), 3.64-3.60 (m, 1H), 3.47-3.42 (m, 1H), 3.33-3.29 (m, 1H), 2.71-2.68 (m, 1H), 2.32 (s, 3H), 2.10-2.06 (m, 1H), 1.83-1.78 (m, 1H); ESI MS m/z 400 [M+H]⁺; HPLC 98.7% (AUC), t_{R}=14.8 min.

### Example 18: (±)-2-(3-((5-Fluoro-2-(trifluoromethyl)phenoxy)methyl)pyrrolidin-1-yl)-6-methylpyrimidine-4-carboxylic Acid (±)-88

Compound (±)-88 was prepared from 5-fluoro-2-(trifluoromethyl)phenol and (±)-tert-butyl 3-((tosyloxy)methyl)pyrrolidine-1-carboxylate (±)-40 according to a similar procedure described for the synthesis of (±)-44: ¹H NMR (400 MHz, DMSO-d₆) δ 7.66-7.62 (m, 1H), 7.20 (d, J=11.2 Hz, 1H), 6.93 (s, 1H), 6.91-6.86 (m, 1H), 4.18-4.10 (m, 2H), 3.73-3.63 (m, 2H), 3.51-3.44 (m, 1H), 3.35-3.32 (m, 1H), 2.76-2.69 (m, 1H), 2.31 (s, 3H), 2.14-2.06 (m, 1H), 1.87-1.79 (m, 1H); ESI MS m/z 400 [M+H]⁺; HPLC 98.0% (AUC), t_{R}=14.7 min.

### Example 19: (±)-2-(3-((2-Fluoro-6-(trifluoromethyl)phenoxy)methyl)pyrrolidin-1-yl)-6-methylpyrimidine-4-carboxylic Acid (±)-89

Compound (±)-89 was prepared from 2-fluoro-6-(trifluoromethyl)phenol and (±)-tert-butyl 3-((tosyloxy)methyl)pyrrolidine-1-carboxylate (±)-40 according to a similar procedure described for the synthesis of (±)-44: ¹H NMR (400 MHz, DMSO-d₆) δ 7.64-7.60 (m, 1H), 7.48 (d, J=8.0 Hz, 1H), 7.29-7.24 (m, 1H), 6.94 (s, 1H), 4.19-4.13 (m, 2H), 3.77-3.73 (m, 1H), 3.67-3.61 (m, 1H), 3.51-3.45 (m, 1H), 3.34-3.34 (m, 1H), 2.77-2.72 (m, 1H), 2.32 (s, 3H), 2.13-2.09 (m, 1H), 1.89-1.82 (m, 1H); ESI MS m/z 400 [M+H]⁺; HPLC 96.7% (AUC), t_{R}=14.8 min.

### Example 20: (k)-2-(3-((5-Methoxy-2-(trifluoromethyl)phenoxy)methyl)pyrrolidin-1-yl)-6-methylpyrimidine-4-carboxylic Acid (±)-90

Compound (±)-90 was prepared from 5-methoxy-2-(trifluoromethyl)phenol and (±)-tert-butyl 3-((tosyloxy)methyl)pyrrolidine-1-carboxylate (±)-40 according to a similar procedure described for the synthesis of (±)-44: ¹H NMR (400 MHz, CDCl₃) δ 7.47 (d, J=8.8 Hz, 1H), 6.94 (s, 1H), 6.75 (s, 1H), 6.58 (d, J=8.4 Hz, 1H), 4.13-4.07 (m, 2H), 3.79 (s, 3H), 3.74-3.64 (m, 2H), 3.51-3.45 (m, 1H), 3.36-3.31 (m, 1H), 2.73-2.70 (m, 1H), 2.31 (s, 3H), 2.12-2.08 (m, 1H), 1.87-1.80 (m, 1H); ESI MS m/z 412 [M+H]⁺; HPLC>99% (AUC), t_{R}=14.6 min.

### Example 21: (k)-2-(3-((3,5-Bis(trifluoromethyl)phenoxy)methyl)pyrrolidin-1-yl)-6-methylpyrimidine-4-carboxylic Acid (±)-91

Compound (±)-91 was prepared from 3,5-bis(trifluoromethyl)phenol and (±)-tert-butyl 3-((tosyloxy)methyl)pyrrolidine-1-carboxylate (±)-40 according to a similar procedure described for the synthesis of (±)-44: ¹H NMR (400 MHz, DMSO-d₆) δ 7.61-7.59 (m, 3H), 6.94 (s, 1H), 4.23-4.14 (m, 2H), 3.76-3.71 (m, 1H), 3.69-3.63 (m, 1H), 3.53-3.47 (m, 1H), 3.42-3.38 (m, 1H), 2.78-2.71 (m, 1H), 2.32 (s, 3H), 2.16-2.08 (m, 1H), 1.89-1.81 (m, 1H); ESI MS m/z 450 [M+H]⁺; HPLC 97.0% (AUC), t_{R}=16.0 min.

### Example 22: (±)-6-Methyl-2-(3-(((4-(trifluoromethyl)pyridin-3-yl)oxy)methyl)pyrrolidin-1-yl)pyrimidine-4-carboxylic Acid (±)-92

Compound (±)-92 was prepared from 4-(trifluoromethyl)pyridin-3-ol and (±)-tert-butyl 3-((tosyloxy)methyl)pyrrolidine-1-carboxylate (±)-40 according to a similar procedure described for the synthesis of (±)-44: ¹H NMR (400 MHz, CDCl₃) δ 8.44 (s, 1H), 8.37 (d, J=4.8 Hz, 1H), 7.43 (d, J=4.8 Hz, 1H), 7.13 (s, 1H), 4.21-4.20 (m, 2H), 3.87-3.78 (m, 2H), 3.65-3.64 (m, 1H), 3.54-3.49 (m, 1H), 2.93-2.86 (m, 1H), 2.44 (s, 3H), 2.29-2.24 (m, 1H), 2.02-1.97 (m, 1H); ESI MS m/z 383 [M+H]⁺; HPLC>99% (AUC), t_{R}=12.6 min.

### Example 23: (±)-6-Methyl-2-(3-(((2-(trifluoromethyl)pyridin-3-yl)oxy)methyl)pyrrolidin-1-yl)pyrimidine-4-carboxylic Acid (k)-93

Compound (±)-93 was prepared from 2-(trifluoromethyl)pyridin-3-ol and (±)-tert-butyl 3-((tosyloxy)methyl)pyrrolidine-1-carboxylate (±)-40 according to a similar procedure described for the synthesis of (±)-44: ¹H NMR (400 MHz, CDCl₃) δ 8.26 (d, J=4.4 Hz, 1H), 7.45-7.42 (m, 1H), 7.34 (d, J=8.4 Hz, 1H), 7.13 (s, 1H), 4.11-4.07 (m, 2H), 3.86-3.85 (m, 2H), 3.63-3.61 (m, 1H), 3.51-3.47 (m, 1H), 2.91-2.84 (m, 1H), 2.44 (s, 3H), 2.28-2.25 (m, 1H), 2.00-1.97 (m, 1H); ESI MS m/z 383 [M+H]⁺; HPLC 99.0% (AUC), t_{R}=12.7 min.

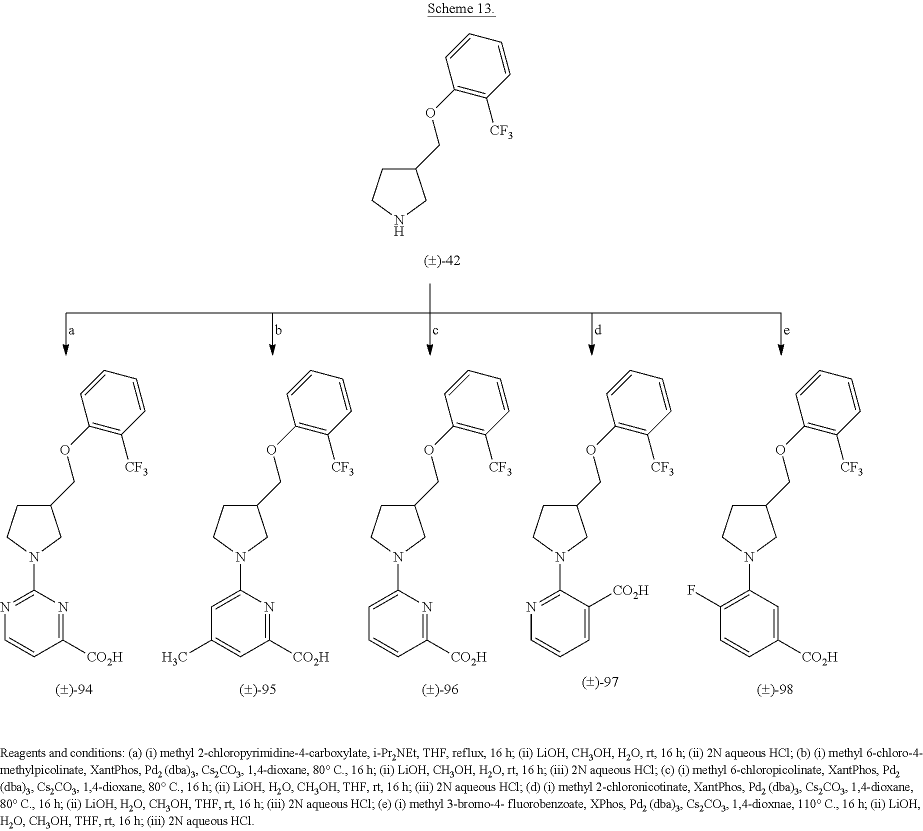 [SMILES: FC(F)(F)c1ccccc1OCC1CCNC1 ; O=C(O)c1ccnc(N2CCC(COc3ccccc3C(F)(F)F)C2)n1 ; Cc1cc(C(=O)O)nc(N2CCC(COc3ccccc3C(F)(F)F)C2)c1 ; O=C(O)c1cccc(N2CCC(COc3ccccc3C(F)(F)F)C2)n1 ; O=C(O)c1cccnc1N1CCC(COc2ccccc2C(F)(F)F)C1 ; O=C(O)c1ccc(F)c(N2CCC(COc3ccccc3C(F)(F)F)C2)c1]

### Example 24: (±)-2-(3-((2-(Trifluoromethyl)phenoxy)methyl)pyrrolidin-1-yl)pyrimidine-4-carboxylic Acid (±)-94

Compound (±)-94 was prepared from methyl 2-chloropyrimidine-4-carboxylate and (±)-3-((2-(trifluoromethyl)phenoxy)methyl)pyrrolidine (±)-42 according to a similar procedure described for the synthesis of (±)-44: ¹H NMR (400 MHz, DMSO-d₆) δ 8.50 (d, J=4.8 Hz, 1H), 7.61-7.57 (m, 2H), 7.26 (d, J=8.4 Hz, 1H), 7.06 (t, J=7.6 Hz, 1H), 7.00 (d, J=5.2 Hz, 1H), 4.15 (brs, 2H), 3.77-3.63 (m, 2H), 3.53-3.47 (m, 1H), 3.37-3.33 (m, 1H), 2.79-2.72 (m, 1H), 2.17-2.09 (m, 1H), 1.90-1.83 (m, 1H); ESI MS m/z 368 [M+H]⁺; HPLC>99% (AUC), t_{R}=14.3 min.

### Example 25: (±)-4-Methyl-6-(3-((2-(trifluoromethyl)phenoxy)methyl)pyrrolidin-1-yl)picolinic Acid (±)-95

Step A: To a mixture of (i)-3-((2-(trifluoromethyl)phenoxy)methyl)pyrrolidine (±)-42 (0.200 g, 0.815 mmol), methyl 6-chloro-4-methylpicolinate (0.151 g, 0.815 mmol), and Cs₂CO₃ (0.796 g, 2.44 mmol) in N₂ degassed anhydrous 1,4-dioxane (10 mL) was added XantPhos (0.153 g, 0.265 mmol) and Pd₂(dba)₃ (74.6 mg, 0.082 mmol). The mixture was heated at 80° C. in a sealed vessel for 16 h. The reaction mixture was allowed to cool to rt and then diluted with H₂O (30 mL) and extracted with EtOAc (3×50 mL). The combined organic extracts were washed with H₂O (3×50 mL), brine (50 mL), dried over Na₂SO₄, filtered, and concentrated under reduced pressure. The resulting residue was chromatographed over silica gel (0% to 50% EtOAc in hexanes) to give (±)-methyl 4-methyl-6-(3-((2-(trifluoromethyl)phenoxy)methyl)pyrrolidin-1-yl)picolinate as an off-white solid (0.180 g, 56%): ¹H NMR (400 MHz, CDCl₃) δ 7.54 (d, J=7.6 Hz, 1H), 7.44 (t, J=8.0 Hz, 1H), 7.23-7.20 (m, 1H), 7.00-6.92 (m, 2H), 6.34 (s, 1H), 4.08-3.99 (m, 2H), 3.89 (s, 3H), 3.76-3.72 (m, 1H), 3.69-3.63 (m, 1H), 3.55-3.49 (m, 1H), 3.40-3.36 (m, 1H), 2.89-2.82 (m, 1H), 2.27 (s, 3H), 2.25-2.18 (m, 1H), 2.00-1.93 (m, 1H); ESI MS m/z 395 [M+H]⁺.

Step B: To a solution of (±)-methyl 4-methyl-6-(3-((2-(trifluoromethyl)phenoxy)methyl)pyrrolidin-1-yl)picolinate (0.190 g, 0.481 mmol) in CH₃OH (6 mL), THF (6 mL), and H₂O (3 mL) was added LiOH (0.115 g, 4.81 mmol) and the mixture stirred at rt for 16 h. The mixture was concentrated under reduced pressure to remove the volatile solvents and the resulting aqueous mixture was diluted with additional H₂O (10 mL) and acidified with 2 N aqueous HCl to pH=3. The acidified mixture was extracted with EtOAc (3×20 mL) and the combined organic extracts were washed with brine (20 mL), dried over Na₂SO₄, filtered, and concentrated under reduced pressure to give (±)-2-(3-((2-(trifluoromethyl)phenoxy)methyl)pyrrolidin-1-yl)pyrimidine-4-carboxylic acid (±)-95 as a white solid (0.136 g, 77%): ¹H NMR (400 MHz, DMSO-d₆) δ 7.57-7.54 (m, 2H), 7.15 (d, J=8.4 Hz, 1H), 7.05-7.01 (m, 2H), 6.18 (s, 1H), 4.02-3.92 (m, 2H), 3.68-3.46 (m, 3H), 3.34-3.32 (m, 1H), 2.64-2.61 (m, 1H), 2.13 (s, 3H), 2.10-2.01 (m, 1H), 1.81-1.68 (m, 1H); ESI MS m/z 381 [M+H]⁺; HPLC>99% (AUC), t_{R}=12.8 min.

### Example 26: (±)-6-(3-((2-(Trifluoromethyl)phenoxy)methyl)pyrrolidin-1-yl)picolinic acid (±)-96

Compound (±)-96 was prepared from methyl 6-chloropicolinate and (±)-3-((2-(trifluoromethyl)phenoxy)methyl)pyrrolidine (±)-42 according to a similar procedure described for the synthesis of (±)-95: ¹H NMR (400 MHz, CDCl₃) δ 7.63-7.54 (m, 2H), 7.46-7.42 (m, 2H), 7.00-6.95 (m, 2H), 6.62-6.60 (m, 1H), 4.10-4.04 (m, 2H), 3.77-3.60 (m, 2H), 3.51-3.39 (m, 2H), 2.92-2.80 (m, 1H), 2.25-2.22 (m, 1H), 2.08-1.96 (m, 1H); ESI MS m/z 367 [M+H]⁺; HPLC 95.8% (AUC), t_{R}=12.7 min.

### Example 27: (±)-2-(3-((2-(Trifluoromethyl)phenoxy)methyl)pyrrolidin-1-yl)nicotinic Acid (±)-97

Compound (±)-97 was prepared from methyl 2-chloronicotinate and (±)-3-((2-(trifluoromethyl)phenoxy)methyl)pyrrolidine (±)-42 according to a similar procedure described for the synthesis of (±)-95: ¹H NMR (400 MHz, CDCl₃) δ 8.40 (d, J=4.4 Hz, 1H), 8.23 (d, J=7.6 Hz, 1H), 7.52 (d, J=7.6 Hz, 1H), 7.47-7.43 (m, 1H), 7.00-6.91 (m, 3H), 4.10-4.02 (m, 2H), 3.61-3.37 (m, 4H), 2.93-2.86 (m, 1H), 2.29-2.21 (m, 1H), 1.98-1.89 (m, 1H); ESI MS m/z 367 [M+H]*; HPLC 98.3% (AUC), t_{R}=12.0 min.

### Example 28: (±)-4-Fluoro-3-(3-((2-(trifluoromethyl)phenoxy)methyl)pyrrolidin-1-yl)benzoic Acid (±)-98

Step A: To a mixture of (i)-3-((2-(trifluoromethyl)phenoxy)methyl)pyrrolidine (±)-42 (0.300 g, 1.22 mmol) and methyl 3-bromo-4-fluorobenzoate (0.342 g, 1.47 mmol) in N₂ degassed 1,4-dioxane was added Cs₂CO₃ (1.2 g, 3.66 mmol), XPhos (58.1 mg, 0.12 mmol), and Pd₂(dba)₃ (37.9 mg, 0.037 mmol). The mixture was stirred at 110° C. in a sealed vessel for 16 h and then allowed to cool to rt. The mixture was concentrated under reduced pressure and the resulting residue was chromatographed over silica gel (0% to 40% EtOAc in hexanes) to give methyl 4-fluoro-3-(3-((2-(trifluoromethyl)phenoxy)methyl)pyrrolidin-1-yl)benzoate as a white solid (0.198 mg, 41%): ¹H NMR (400 MHz, CDCl₃) δ 7.54 (d, J=7.6 Hz, 1H), 7.45 (t, J=7.6 Hz, 1H), 7.37-7.32 (m, 2H), 7.00-6.95 (m, 3H), 4.04 (d, J=7.2 Hz, 2H), 3.85 (s, 3H), 3.64-3.59 (m, 1H), 3.52-3.46 (m, 2H), 3.40-3.35 (m, 1H), 2.85-2.81 (m, 1H), 2.22-2.17 (m, 1H), 1.93-1.88 (m, 1H); ESI MS m/z 398 [M+H]⁺.

Step B: To a solution of methyl 4-fluoro-3-(3-((2-(trifluoromethyl)phenoxy)methyl)pyrrolidin-1-yl)benzoate (0.120 g, 0.63 mmol) in a mixture of CH₃OH (4 mL), THF (4 mL), and H₂O (2 mL) was added LiOH (0.144 g, 6.04 mmol). The mixture stirred at rt for 16 h and was concentrated under reduced pressure to remove the volatile solvents. The resulting aqueous layer was diluted with H₂O (50 mL) and acidified with 2 N aqueous HCl to pH=3. The aqueous mixture was extracted with EtOAc (3×50 mL) and the combined organic extracts were washed with brine, dried over Na₂SO₄, filtered, and concentrated under reduced pressure to give 4-fluoro-3-(3-((2-(trifluoromethyl)phenoxy)methyl)pyrrolidin-1-yl)benzoic acid (±)-98 as a white solid (78.0 mg, 67%): δ 7.59-7.55 (d, J=7.6 Hz, 2H), 7.24-7.20 (m, 3H), 7.13-7.02 (m, 2H), 4.13-4.07 (m, 2H), 3.52-3.48 (m, 1H), 3.39 (brs, 2H), 3.29-3.25 (m, 1H), 2.73-2.70 (m, 1H), 2.11-2.08 (m, 1H), 1.81-1.76 (m, 1H); ESI MS m/z 384 [M+H]⁺; HPLC 98.5% (AUC), t_{R}=16.1 min.

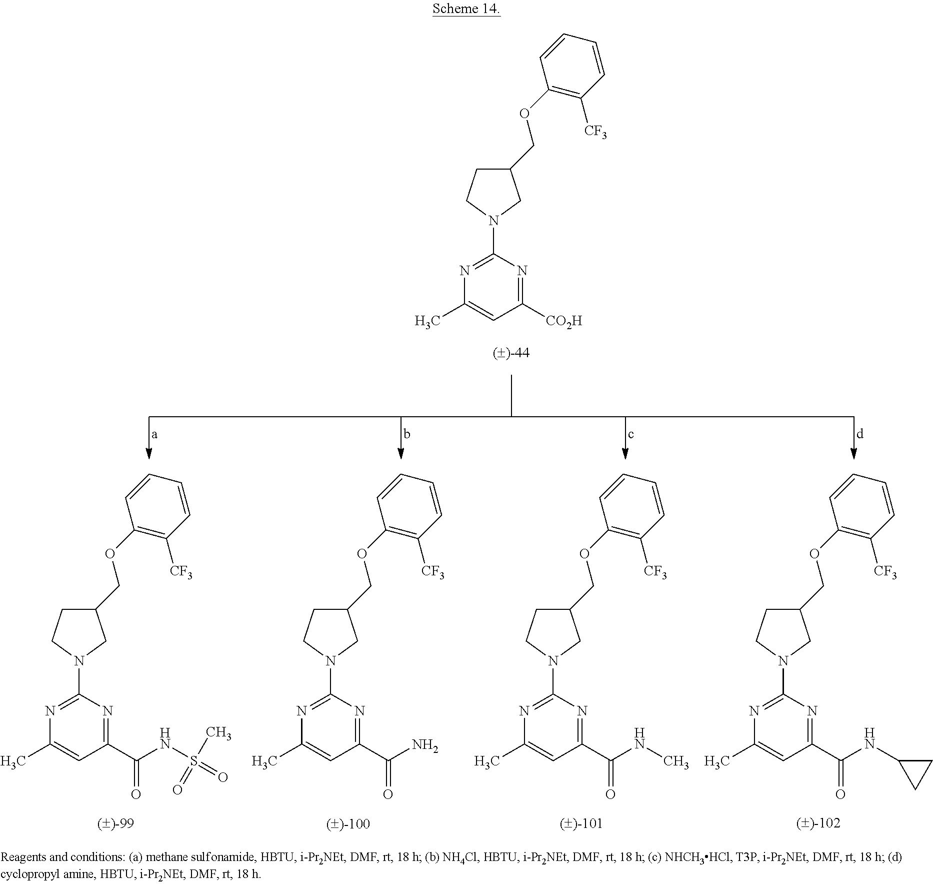

### Example 29: (±)-6-Methyl-N-(methylsulfonyl)-2-(3-((2-(trifluoromethyl)phenoxy)methyl)pyrrolidin-1-yl)pyrimidine-4-carboxamide (±)-99

Step A: To a mixture of 6-methyl-2-(3-((2-(trifluoromethyl)phenoxy)methyl)pyrrolidin-1-yl)pyrimidine-4-carboxylic acid (±)-44 (50.0 mg, 0.131 mmol), HBTU (74.5 mg, 0.197 mmol), and i-Pr₂NEt (0.08 mL, 0.393 mmol) in DMF (4 mL) was added methanesulfonamide (19.1 mg, 0.197 mmol). The resulting solution was stirred at rt for 18 h under an atmosphere of N2. The mixture was diluted with H₂O (10 mL) and extracted with EtOAc (3×20 mL). The combined organic extracts were washed with H₂O (3×20 mL) and brine, dried over Na₂SO₄, filtered, and concentrated under reduced pressure. The resulting crude residue was chromatographed over silica gel (0% to 80% EtOAc in hexanes) to give 6-methyl-N-(methylsulfonyl)-2-(3-((2-(trifluoromethyl)phenoxy)methyl)pyrrolidin-1-yl)pyrimidine-4-carboxamide (±)-99 as a white solid (30.0 mg, 50%): ¹H NMR (400 MHz, acetone-d₆) δ 10.36 (brs, 1H), 7.61-7.57 (m, 2H), 7.27-7.25 (m, 1H), 7.10-7.06 (m, 2H), 4.23 (brs, 2H), 3.92-3.82 (m, 2H), 3.65-3.49 (m, 2H), 3.34 (s, 3H), 2.92-2.82 (m, 2H), 2.42 (s, 3H), 2.25-2.22 (m, 1H); ESI MS m/z 459 [M+H]⁺; HPLC 98.4% (AUC), t_{R}=15.9 min.

### Example 30: (±)-6-Methyl-2-(3-((2-(trifluoromethyl)phenoxy)methyl)pyrrolidin-1-yl)pyrimidine-4-carboxamide (±)-100

Compound (±)-100 was prepared from NH₄Cl and 6-methyl-2-(3-((2-(trifluoromethyl)phenoxy)methyl)pyrrolidin-1-yl)pyrimidine-4-carboxylic acid (±)-44 according to a similar procedure described for the synthesis of (±)-99: ¹H NMR (400 MHz, CDCl₃) δ 7.71 (brs, 1H), 7.55 (d, J=8.0 Hz, 1H), 7.45 (t, J=7.6 Hz, 1H), 7.14 (s, 1H), 7.00-6.94 (m, 2H), 5.57 (brs, 1H), 4.06-4.02 (m, 2H), 3.88-3.83 (m, 1H), 3.79-3.73 (m, 1H), 3.63-3.57 (m, 1H), 3.51-3.47 (m, 1H), 2.89-2.84 (m, 1H), 2.40 (s, 3H), 2.26-2.19 (m, 1H), 1.99-1.94 (m, 1H); ESI MS m/z 381 [M+H]⁺; HPLC>99% (AUC), t_{R}=14.3 min.

### Example 31: (±)-N,6-dimethyl-2-(3-((2-(trifluoromethyl)phenoxy)methyl)pyrrolidin-1-yl)pyrimidine-4-carboxamide (±)-101

Step A: To a solution of 6-methyl-2-(3-((2-(trifluoromethyl)phenoxy)methyl)pyrrolidin-1-yl)pyrimidine-4-carboxylic acid (±)-44 (50.0 mg, 0.131 mmol), T₃P (50% w/w in CH₂Cl₂) (83.4 mg, 0.262 mmol), and i-Pr₂NEt (0.2 mL, 1.05 mmol) in CH₂Cl₂ (3 mL) was added methylamine hydrochloride (44.0 mg, 0.393 mmol). The mixture stirred at ambient temperature for 18 h and was then concentrated under reduced pressure. The resulting residue was chromatographed over silica gel (0% to 60% EtOAc in hexane) to give N,6-dimethyl-2-(3-((2-(trifluoromethyl)phenoxy)methyl)pyrrolidin-1-yl)pyrimidine-4-carboxamide (±)-101 as a white solid (40.0 mg, 77%): ¹H NMR (400 MHz, acetone-d₆) δ 8.25 (brs, 1H), 7.62-7.59 (m, 2H), 7.26 (d, J=8.0 Hz, 1H), 7.08-7.05 (m, 2H), 4.23-4.19 (m, 2H), 3.88-3.83 (m, 1H), 3.79-3.74 (m, 1H), 3.60-3.58 (m, 1H), 3.50-3.48 (m, 1H), 2.88 (s, 3H), 2.87-2.82 (m, 1H), 2.36 (s, 3H), 2.22-2.19 (m, 1H), 2.02-2.01 (m, 1H); ESI MS m/z 395 [M+H]⁺; HPLC>99% (AUC), t_{R}=14.7 min.

### Example 32: (±)-N-Cyclopropyl-6-methyl-2-(3-((2-(trifluoromethyl)phenoxy)methyl)pyrrolidin-1-yl)pyrimidine-4-carboxamide (±)-102

Compound (±)-102 was prepared from cyclopropylamine and 6-methyl-2-(3-((2-(trifluoromethyl)phenoxy)methyl)pyrrolidin-1-yl)pyrimidine-4-carboxylic acid ((±)-44 according to a similar procedure described for the synthesis of (±)-100: ¹H NMR (400 MHz, acetone-d₆) δ 8.17 (brs, 1H), 7.62-7.57 (m, 2H), 7.26 (d, J=8.4 Hz, 1H), 7.10-7.04 (m, 2H), 4.21 (brs, 2H), 3.87-3.82 (m, 1H), 3.76-3.72 (m, 1H), 3.58-3.55 (m, 1H), 3.49-3.44 (m, 1H), 2.88-2.78 (m, 2H), 2.36 (s, 3H), 2.23-2.13 (m, 1H), 2.02-1.95 (m, 1H), 0.75-0.73 (m, 2H), 0.58 (brs, 2H); ESI MS m/z 421 [M+H]⁺; HPLC>99% (AUC), t_{R}=15.3 min.

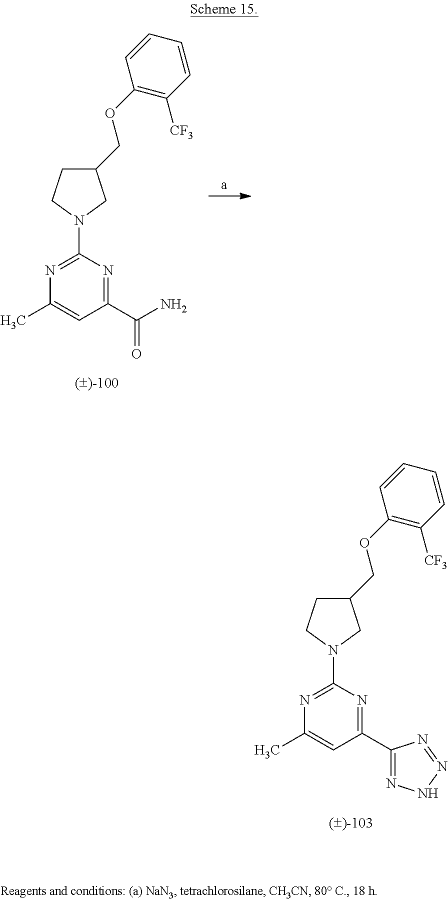 [SMILES: Cc1cc(C(N)=O)nc(N2CCC(COc3ccccc3C(F)(F)F)C2)n1 ; Cc1cc(-c2nn[nH]n2)nc(N2CCC(COc3ccccc3C(F)(F)F)C2)n1]

### Example 33: (±)-4-Methyl-6-(2H-tetrazol-5-yl)-2-(3-((2-(trifluoromethyl)phenoxy)methyl)pyrrolidin-1-yl)pyrimidine (±)-103

Step A: A mixture of 6-methyl-2-(3-((2-(trifluoromethyl)phenoxy)methyl)pyrrolidin-1-yl)pyrimidine-4-carboxamide (±)-100 (0.200 g, 0.526 mmol), NaN₃ (0.142 g, 0.375 mmol), and tetrachlorosilane (98.5 mg, 0.579 mmol) in CH₃CN (4 mL) stirred at 80° C. for 18 h in a sealed vessel. The reaction mixture was allowed to cool to rt and diluted with saturated NaHCO₃ (5 mL). The aqueous mixture was extracted with CHCl₃ (3×50 mL) and the combined organic extracts were washed with brine (50 mL), dried over Na₂SO₄, filtered, and concentrated under reduced pressure. The resulting residue was chromatographed over silica gel (0% to 10% CH₃OH in CH₂Cl₂) to give 4-methyl-6-(2H-tetrazol-5-yl)-2-(3-((2-(trifluoromethyl)phenoxy)methyl)pyrrolidin-1-yl)pyrimidine (±)-103 as a white solid (66.0 mg, 30%): ¹H NMR (400 MHz, acetone-d₆) δ 7.62-7.58 (m, 2H), 7.28-7.25 (m, 2H), 7.08 (t, J=7.6 Hz, 1H), 4.28-4.21 (m, 3H), 3.91-3.86 (m, 1H), 3.80-3.74 (m, 1H), 3.653.58 (m, 1H), 3.52-3.47 (m, 1H), 2.92-2.85 (m, 1H), 2.42 (s, 3H), 2.27-2.21 (m, 1H); ESI MS m/z 406 [M+H]⁺: HPLC 97.4% (AUC), t_{R}=14.6 min.

### Example 34: In Vitro Binding of Compounds to RBP4

Compound binding to RBP4 was assessed in the radiometric scintillation proximity (SPA) assay that was previously described (Cioffi, C. L. et al. 2014; Cioffi, C. L. et al. 2015; Cioffi, C. L. et al. 2019). The assay measured competitive displacement of radiolabeled retinol from native RBP4 purified from human urine (Fitzgerald, 30R-AR022L). The protein was biotinylated using the EZ-link Sulfo-NHS-LC-Biotinylation kit from ThermoFisher (Cat #21335) as recommended by the manufacturer. Binding assays were implemented in a final volume of 100 μL in SPA buffer (1×PBS, pH 7.4, 1 mM EDTA, 0.1% BSA, 0.5% CHAPS). The assay reaction included a radiligand, 10 nM ³H-retinol (48.7 Ci/mmol; PerkinElmer, Waltham, MA), along with the 0.3 mg/well Streptavidin-PVT beads (PerkinElmer, RPNQ0006) and 50 nM biotinylated human RBP4. Unlabeled retinol (Sigma, cat #95144) at 20 μM was added to control wells to assess a nonspecific binding. Radioactivity counts were measured using CHAMELEON plate reader (Hidex Oy, Turku, Finland) after 16 h of incubation at room temperature (rt) with mild shaking.

### Example 35: Assessment of Antagonistic Activity in the HTRF RBP4-TTR Interaction Assay

The ability of analogues to act as antagonists of all-trans-retinol-dependent RBP4-TTR interaction was measured in the HTRF (Homogenous Time-Resolved Fluorescence) assay as we described previously (Cioffi, C. L. et al. 2014; Cioffi, C. L. et al. 2015; Cioffi, C. L. et al. 2019). Untagged TTR (Calbiochem, cat #529577) and Maltose-Binding Protein-tagged RBP4 expressed in E. coli were used in this assay. HTRF Cryptate labeling kit from CisBio (Cisbio, cat #62EUSPEA, Bedfored, MA) was used to label TTR with Eu³⁺ Cryptate. The assay was performed in a final assay volume of 16 μl in the buffer that contained 10 mM Tris-HCl pH 7.5, 1 mM DTT, 0.05% NP-40, 0.05% Prionex, 6% glycerol, and 400 mM KF. Other components of the reaction mix included 60 nM MBP-RBP4, 5 nM TTR-Eu, 26.7 nM of anti-MBP antibody conjugated with d2 (Cisbio, cat #61MBPDAA), and 1 μM all-trans retinol (Sigma, cat #95144). All of the reactions were performed under dim red light in the dark. The plates were read in the SpectraMax M5e Multimode Plate Reader (Molecular Devices, Sunnyvale, CA) after the overnight incubation at 4° C. Fluorescence was excited at 337 nm; emission was measured at 668 and 620 nm with 75 μs counting delay. The HTRF signal was expressed as the ratio of fluorescence intensity: Flu₆₆₈/Flu₆₂₀×10,000.

### Example 36: Fluorescence Polarization TTR Tetramer Binding Assay

Compound binding to TTR was assessed in the fluorescence polarization assay. The assay measured competitive displacement of the fluorescent probe, FITC-diclofenac, from TTR isolated from human plasma (Clabiochem-Millipore, cat. No. 52957). FITC-diclofenac was synthesized at LeadGen Labs, LLC following the published procedure (Alhamadsheh, M. M. et al. 2011). Each well contained 200 nM TTR and 100 nM FITC-diclofenac in the FP buffer (10 mM Tris-HCl pH 7.5, 150 mM NaCl, 0.01% CHAPS, 0.01% Prionex) along with test compounds. Nonspecific binding was determined in the presence of 500 μM unlabeled diclofenac (Sigma-Aldrich). Reactions with test compounds were incubated overnight at 4° C. and FP was measured on SpectramaxM5e plate reader (Molecular Devices).

TABLE 1
RBP4 SPA binding affinity, RBP4-TTR HTRF, and TTR fluorescence
polarization data for select compounds.

| | RBP4 SPA^{a} | RBP4-TTR HTRF^{b} | TTR FP^{c} |
|---|---|---|---|
| Compound | IC₅₀ (μM)^{d} | IC₅₀ (μM)^{d} | IC₅₀ (μM)^{d} |
| 21 | 2.44 | 18.7 | 4.0 |
| (±)-27 | >3 | ND | 8.8 |
| 32 | >3 | ND | 13.5 |
| 38 | 0.8 | 6.5 | 26 |
| (±)-44 | 0.08 | 0.25 | 2.85 |
| (R)-50 | 0.065 | 0.24 | 3.2 |
| (S)-56 | 0.15 | 0.75 | 3.9 |
| (±)-62 | 0.48 | 15 | 1.1 |
| (±)-67 | 0.26 | ND | 1.6 |
| (±)-71 | 0.23 | 1.3 | 10.5 |
| (±)-76 | 0.9 | 7.5 | 3.5 |
| (±)-80 | >3 | ND | 13 |
| (±)-83 | 0.13 | 0.45 | >30 |
| (±)-84 | 0.16 | 0.54 | >30 |
| (±)-85 | 0.21 | ND | >30 |
| (±)-86 | 0.18 | ND | 3.9 |
| (±)-87 | 0.24 | 0.87 | 3.9 |
| (±)-88 | 0.13 | 0.3 | 13 |
| (±)-89 | 0.3 | 1 | 1.9 |
| (±)-90 | >3 | ND | >30 |
| (±)-91 | 1.6 | ND | >30 |
| (±)-92 | 0.52 | 3.7 | >30 |
| (±)-93 | 0.66 | ND | >30 |
| (±)-94 | 1.67 | ND | 11 |
| (±)-95 | 0.13 | 0.54 | 7.4 |
| (±)-96 | >3 | ND | 25 |
| (±)-97 | 0.68 | 1.3 | 4.9 |
| (±)-98 | 2.35 | ND | 1.88 |
| (±)-99 | 0.081 | 0.7 | 4.2 |
| (±)-100 | 0.062 | 2.3 | >30 |
| (±)-101 | 0.089 | >30 | >30 |
| (±)-102 | 0.062 | 19 | 4.1 |
| (±)-103 | 0.086 | 1.6 | 1.26 |

^{a}IC₅₀ values for the SPA assay obtained in the presence of a fixed, 10 nM concentration of ³H-retinol.
^{b}IC₅₀ values for the HTRF assay obtained in the presence of 1 □M concentration of retinol.
^{c}IC₅₀ values for the fluorescence polarization (FP) assay obtained in the presence of a fixed, 25 μM concentration of fluorescein isothiocyanate (FITC)-coupled TTR FP probe.
^{d}For compounds tested multiple times (more than twice) the IC₅₀ data is represented as the mean ± standard deviation. For those compounds that were only tested twice, the IC₅₀ data is shown as the mean of two independent experiments and not as the mean ± standard deviation.
ND = Not determined.

### Example 37: TTR Aggregation Assay

The ability of test compounds to prevent TTR aggregation was evaluated under the acidic conditions that favor TTR aggregation and fibril formation. A 2 μl solution of 167 μM human TTR (ACROBiosystems #H5223) was incubated with 7 μl 50 mM sodium acetate pH 4.0 (Sigma #S7545), 100 mM KCl (Sigma #S5405) in the presence or absence of 1 μl TTR inhibitor for 72 h at 37° C. At the end of the incubation, 3.5 μl 500 mM sodium phosphate (Sigma #S5136) buffer pH=8.0 was added to each sample for neutralization and 0.6 μl 5% CHAPS (Sigma #C5070) as a detergent to prevent reassociation of protein. The cross-linking was performed by adding 1.5 μl 5% glutaraldehyde solution (Sigma #G6257). After 4 min, the reaction was stopped by the addition of 2.5 μl freshly made 5% NaBH4. Samples were subjected to TTR western blotting with prealbumin antibodies (1:500; Dako #A0002). Band intensity for TTR monomer and TTR aggregates was quantified from scanned images of the blots.

### In Vitro ADME Assay Information

### Example 38: Kinetic Solubility Assay

Kinetic aqueous solubility determination for compound (±)-44 in PBS (pH 7.4) was conducted by Eurofins using UV detection (230 nm). Aqueous solubility (μM) was determined by comparing the peak area of the principal peak in a calibration standard (200 μM) containing organic solvent (methanol/water, 60/40, v/v) with the peak area of the corresponding peak in a buffer sample. In addition, chromatographic purity (%) was defined as the peak area of the principal peak relative to the total integrated peak area in the HPLC chromatogram of the calibration standard. A chromatogram of the calibration standard of each test compound, along with a UV/VIS spectrum with labeled absorbance maxima, was generated.

### Standards for the Kinetic Solubility Study:

Metoprolol—192.6 μM
Rifampicin—200 μM
Ketoconazole—152.8 μM
Phenytoin—101.8 1 μM
Simvastatin—14.2 μM
Diethylstilbesterol—7.0 μM
Tamoxifen—1.9 μM

### Example 39: CYP450 Inhibition Assay

Inhibition potential (IC₅₀ values) results for compound (±)-44 against the human cytochrome P450 (CYP) isoforms 2C9, 2C19, 2D6, and 3A4. Each recombinant human CYP isoform was tested with a standard positive and negative control, using fluorometric detection for measuring CYP activity. The measured IC₅₀ values for the respective standard inhibitors were all within expected ranges for each isoform (see below).

### IC₅₀ Concentrations of Standard CYP Inhibitors:

CYP Inhibitor IC₅₀ (μM):
2C9 Sulfaphenazole IC₅₀=3.4 μM
2C19 Tranylcypromine IC₅₀=2.8 μM
2D6 Quinidine IC₅₀=0.058 μM
3 Å4 Ketoconazole IC₅₀=0.0084 μM

Pre-formulated NADPH regenerating solutions, recombinant CYP isoforms 2C19 and 3 Å4 (Lot #3007790 and 2276593 respectively), 3-[2-(N,N-diethyl-N-methylamino)ethyl]-7-methoxy-4-methylcoumarin (AMMC), 3-cyano-7-ethoxycoumarin (CEC) and 7-benzyloxy-4-trifluoromethylcoumarin (BFC) were obtained from Corning Life Sciences (Bedford, MA). Recombinant CYP isoform 2D6 (Lot #49242) was obtained from Invitrogen (Carlsbad, CA). CYP isoform 2C9 (Lot #0446966-1) was obtained from Cayman Chemical (Ann Arbor, MI). 7-methoxy-4-trifluoromethylcoumarin (MFC), trans-2-phenylcyclopropylamine HCl (TCP), sulfaphenazole (SFZ), ketoconazole (KTZ) and quinidine (QDN) were obtained from Sigma (St. Louis, MO). All solvents and buffers were obtained from commercial sources and used without further purification.

### Methods:

Test compound was prepared as a 10 mM stock solution in acetonitrile. Four human P450 isoforms cDNA-expressed in insect cell microsomes (CYP2C9, CYP2C19, CYP2D6, and CYP3A4) were tested for inhibition by test compound using fluorescence-based assays. Nine serial dilutions (concentrations from 0-100 μM) using each test compound stock solution were prepared in black microtiter plates, in duplicate. This dilution series was incubated at 37° C. with the individual CYP isoforms and a standard fluorogenic probe substrate for each respective isoform. The concentration of the probe substrate added was at or near the Km value for each CYP isoform. Reaction mixtures contained potassium phosphate buffer, pH 7.4 and the NADPH-regenerating system. The final reaction volume was 0.20 mL and the reaction was terminated with 75 μL of stop solution (0.5 M Tris base in acetonitrile) after the appropriate incubation time (15-45 minutes). Fluorescence measurements were made at the appropriate excitation and emission wavelengths. Duplicate control wells with no test compound, duplicate blank wells containing stop solution prior to adding isoform, and a dilution series in duplicate containing a standard inhibitor for each isoform were also conducted. IC50 values were calculated using a non-linear regression of the data using the four-parameter logistic model (dose response equation) fit with XLFit 5.2 from IDBS Software (Emeryville, CA), supported by linear interpolation of data points at concentrations indicating inhibition levels approximately 50% of the uninhibited rate.

### Example 40: Plasma Protein Binding Assay

Plasma protein binding (PPB) for compounds determination for compound (±)-44 in PBS (pH 7.4) was conducted by Eurofins using equilibrium dialysis of plasma with HPLC-UV/Vis detection.

### Mean Plasma Protein Binding of Control Propranolol in Human, Rat (Sprague Dawley), Mouse (CD-1), and Dog (Beagle) Plasma

The peak areas of the test compound in the buffer and test samples were used to calculate percent binding and recovery according to the following formulas:

$$Protein binding ( \% ) = \frac{{Area}_{p} - {Area}_{c}}{{Area}_{p}} \times 100$$

$$Recovery ( \% ) = \frac{{Area}_{p} + {Area}_{b}}{{Area}_{c}} \times 100$$

### Where:

Areaₚ=Peak area of analyte in protein matrix
Area_{b}=Peak area of analyte in buffer
Area_{c}=Peak area of analyte in control sample

### Example 41: Metabolic Stability

### Metabolic Stability in Microsomes

The results of metabolic stability determinations for novel compounds and testosterone (positive control) were conducted in the presence of human, rat, mouse, and monkey liver microsomes. Values shown are percent of parent remaining after a 30 minute incubation. All measurements were done in duplicate. Assay results for testosterone were within an acceptable range.

### Metabolic Clearance in Microsomes

Mixed-gender human liver microsomes (Lot #1710084), male Sprague-Dawley rat liver microsomes (Lot #1610290), male CD-1 mouse liver microsomes (Lot #1710069), and male Cynomolgous liver microsomes (Lot #1510193) were purchased from XenoTech. The reaction mixture, minus NADPH, was prepared as described below. The test article was added into the reaction mixture at a final concentration of 1 μM. The control compound, testosterone, was run simultaneously with the test article in a separate reaction. An aliquot of the reaction mixture (without cofactor) was equilibrated in a shaking water bath at 37° C. for 3 minutes. The reaction was initiated by the addition of cofactor, and the mixture was incubated in a shaking water bath at 37° C. Aliquots (100 μL) were withdrawn at 0, 10, 20, 30, and 60. Test article and testosterone samples were immediately combined with 400 μL of ice-cold 50/50 acetonitrile (ACN)/H₂O containing 0.1% formic acid and internal standard to terminate the reaction. The samples were then mixed and centrifuged to precipitate proteins. All samples were assayed by LC-MS/MS using electrospray ionization. The peak area response ratio (PARR) to internal standard was compared to the PARR at time 0 to determine the percent remaining at each time point. Half-lives were calculated using GraphPad software, fitting to a single-phase exponential decay equation.

TABLE 2
In Vitro ADMET Profile for (±)-44.

| | | | | | Liver Microsomal Stability | | | | | | | | | |
|---|---|---|---|---|---|---|---|---|---|---|---|---|---|---|
| | Microsomal CLᵢₙₜ | | | | (% remaining at 30 min)^{c} | | | | CYP Inhibition | | | | | |
| | (μL/min/mg)^{b} | | | | | | | cyno | (% inhibition at 10 μM) | hERG^{d} | PPARγ | % PPB^{e} | | |
| Solubility^{a} | H | R | M | cyno | HLM | RLM | MLM | LM | 2C9, 2C19, 2D6, 3A4 | (IC₅₀) | (IC₅₀) | H | R | M |

| | | | | | | | | | | | |
|---|---|---|---|---|---|---|---|---|---|---|---|
| 187.4 μM | <0.0231 | 105 | 93 | 95 | 103 | 2C9 - 0% | >30 | >100 | 99.8 | 96 | 96 |

| | | |
|---|---|---|
| 2C19 - (−)8% | μM | μM |
| 2D6 - 18.1% | | |
| 3A4 - (−)12.2% | | |

^{a}Kinetic solubility measured in PBS (pH = 7.4).
^{b}Microsomal intrinsic clearance (CLᵢₙₜ); H = human; R = rat; M = mouse; cyno = cynomolgus monkey.
^{c}Liver microsomal metabolic stability, % of parent drug remaining after a 30 minute incubation in the presence of the microsomes; HLM = human liver microsomes; RLM = rat liver microsomes; MLM = mouse liver microsomes; cyno LM = cynomolgus monkey liver microsomes.
^{d}CiPA hERG QPatch Assay; compounds were tested (n = 2) in a five-point concentration-response study.
^{e}% PPB = plasma protein binding; H = human, R = rat, M = mouse.

### Example 42: In Vivo PK Assay

### Mouse PK Study Information and Data

Drug naïve adult male CD-1 mice were administered a single dose administration of the test article by intravenous (IV) or oral gavage (PO) dose routes.
Testing Facility and Test Site: Absorption Systems, LLC, 436 Creamery Way, Suite 600, Exton, PA 19341-2556

### Test Article and Vehicle Information:

IV dosing vehicles: 3% DMA/45% PEG300/12% ethanol/40% sterile water
PO dosing vehicle: 2% Tween 80 in 0.9% saline
Dose formulation: The dose formulation was prepared by the step-wise addition (in the order listed) of the individual components of the vehicle to a weighed quantity of test compound in a volume that yielded the desired final concentration. Each formulation was prepared by mixing a weighed quantity of test compound with the appropriate volume of vehicle.
Dosing Solution Analysis: The dosing solutions were analyzed by LC-MS/MS. The dosing solutions were diluted into mouse blood and analyzed in triplicate. All concentrations are expressed as mg/mL of the free base. The nominal dosing level was used in all calculations for Group 1.

### Test System:

Species and strain: mouse; male CD-1
Mean weight: 0.034 kg for the IV arm; 0.027 kg for PO arm
Number: 6 animals total (three animals per dosing group (Group 1 (IV) and Group 2 (PO)), per study)
COMPLIANCE: This non-clinical study followed established practices and standard operating procedures of Absorption Systems as well as the study protocol. This study was exploratory in nature and was not conducted in accordance with the principles set forth in the United States Food and Drug Administration (FDA) Good Laboratory Practice (GLP) Regulations, 21 Code of Federal regulations (CFR) Part 58. The report is archived in a validated scientific data management system. Electronic signatures comply with the regulation 21 CFR Part 11.

### Experimental Design:

Blood was collected from mice at pre-dose and at 5, 15 and 30 min, and 1, 2, 4, 8, 24 and 48 h post-dose. Hemolyzed blood samples were extracted by protein precipitation using acetonitrile. Following protein extraction with acetonitrile, compound levels were measured by LC-MS/MS. Pharmacokinetic parameters were calculated from the time course of the blood concentrations. Pharmacokinetic parameters were determined with Phoenix WinNonlin (v8.0) software using a non-compartmental model. The maximum blood concentrations (CO) after IV dosing were estimated by extrapolation of the first two time points back to t=0. The maximum blood concentration (Cmax) and the time to reach maximum blood concentration (tmax) after PO dosing were observed from the data. The area under the time concentration curve (AUC) was calculated using the linear trapezoidal rule with calculation to the last quantifiable data point, and with extrapolation to infinity if applicable. Blood half-life (t ½) was calculated from 0.693/slope of the terminal elimination phase. Mean residence time, MRT, was calculated by dividing the area under the moment curve (AUMC) by the AUC. Clearance (CL) was calculated from dose/AUC. Steady-state volume of distribution (Vss) was calculated from CL*MRT. Bioavailability was determined by dividing the individual dose normalized PO AUC∞ values by the average dose-normalized IV AUC∞ value. Any samples below the limit of quantitation (1.00 ng/mL) were treated as zero for pharmacokinetic data analysis.

TABLE 3
In Vivo PK Data for Analogue (±)-44 Following IV and PO Administration in mice.

| | | C₀^{a} | CL^{b} | t_{1/2}^{c} | Vₛₛ^{d} | AUCₗₐₛₜ^{e} | AUC_{INF}^{f} | |
|---|---|---|---|---|---|---|---|---|
| Route | Dose | (ng/ml) | (L/hr/kg) | (h) | (L/kg) | (hr · ng/mL) | (hr · ng/mL) | % Fⁱ |
| IV | 2 mg/kg | 9129 | 0.0499 | 8.6 | 0.518 | 39523 | 40336 | NA |
| | | (661) | (0.005) | (1.15) | (0.069) | (3665) | (4021) | |
| | | Cₘₐₓ^{g} | Tₘₐₓ^{h} | t_{1/2}^{c} | Vₛₛ^{d} | AUCₗₐₛₜ^{e} | AUC_{INF}^{f} | |
| Route | Dose | (ng/ml) | (h) | (h) | (mL/kg) | (hr · ng/mL) | (hr · ng/mL) | % Fⁱ |
| PO | 5 mg/kg | 3033 | 0.83 | 9.9 | NA | 50400 | 52439 | 52.0 |
| | | (692) | (0.29) | (2.0) | | (5898) | (7214) | (7.15) |

Data are represented as the mean with standard deviation in parentheses (mean (SD)). Dosing groups consisted of three drug naïve adult male CD-1 mice. IV administration: Test article was administered at the 2 mg/kg dose; test article vehicle = 3% DMA/45% PEG300/12% Ethanol/40% Sterile water; PO administration: test article was administered at the 5 mg/kg dose, vehicle = 2% Tween 80 in 0.9% saline.
^{a}Observed initial concentration of compound in blood at time zero.
^{b}Total body clearance.
^{c}Apparent half-life of the terminal phase of elimination of compound from blood.
^{d}Volume of distribution at steady state.
^{e}Area under the blood concentration versus time curve from 0 to the last time point that compound was quantifiable in blood.
^{f}Area under the blood concentration versus time curve from 0 to infinity.
^{g}Maximum observed concentration of compound in blood.
ⁱTime of maximum observed concentration compound in blood.
^{f}Bioavailability; F = (AUC_{INFpo} × Doseᵢᵥ) ÷ AUC_{INFiv} × Doseₚₒ).

### Example 43: Serum RBP4 Measurement Assay

Blood samples were collected from a tail vein. Whole blood was drawn into a centrifuge tube and was allowed to clot at room temperature for 30 minutes followed by centrifugation at 2000 g for 15 minutes at 48° C. to collect serum. Aliquots of plasma samples collected in the mouse pharmacokinetic study were analyzed for the RBP4 concentration using the RBP4 (mouse/rat) dual ELISA kit (AdipoGen, San Diego, CA) following the manufacturer's instructions. In adi-hRBP4 transgenic mouse experiments, blood samples were collected from a tail vein. Whole blood was drawn into a centrifuge tube and was allowed to clot at room temperature for 30 min followed by centrifugation at 2000 g for 15 min at +4° C. to collect serum. Mouse serum RBP4 (produced predominantly in the liver) was measured using the RBP4 (mouse/rat) dual ELISA kit (AdipoGen, San Diego, CA; catalog number AG-45A-0012YTP-KI01)

### Animal Care and Use Statement:

All procedures in this protocol are in compliance with the U.S. Department of Agriculture's (USDA) Animal Welfare Act (9 CFR Parts 1, 2, and 3); the Guide for the Care and Use of Laboratory Animals, Institute of Laboratory Animal Resources, National Academy Press, Washington, D.C., 1996; and the National Institutes of Health, Office of Laboratory Animal Welfare. Whenever possible, procedures in this study are designed to avoid or minimize discomfort, distress, and pain to animals.

### Example 44. Effect of (±)-44 on Accumulation of N-Retinylidene-N-Retinylethanolamine (A2E) in Eyes of the Abca4^{-/-} Mice

(±)-44 was formulated into a Picolab 5053 chow to ensure a daily dosing of 25 mg/kg of (±)-44. Long-term 12-week dosing of the compound formulated into a chow was conducted in Abca4^{-/-} mice. The age-matched control group of wild-type 12951/SvLmJ mice was kept on a standard Picolab 5053 chow. The age-matched reference group of mice was used for defining the basal level of A2E in mice in the absence of the Abca4 ablation. Blood samples for assessing the serum levels of RBP4 were collected from (±)-44-treated and control chow-treated Abca4^{-/-} mice at pre-dose and after 12 weeks of treatment.

Following the 12 weeks of dosing, the eyecups of treated and untreated Abca4^{-/-} mice as well as the eyecups of the reference wild type mice were collected for the quantitative A2E analysis.

### Example 45. Effect of (±)-44 on Accumulation of N-Retinylidene-N-Retinylethanolamine (A2E) in Eyes of the Double Knock-Out Abca4^{-/-}/Rdh8^{-/-} Mice

(±)-44 was formulated into a Picolab 5053 chow to ensure a daily dosing of 25 mg/kg of (±)-44. Long-term 10-week dosing of the compound formulated into a chow was conducted in Abca4^{-/-}/Rdh8^{-/-} mice. The age-matched control group of wild-type C57BL/6J mice was kept on a standard Picolab 5053 chow. The age-matched reference group of mice was used for defining the basal level of A2E in mice in the absence of the Abca4 and Rdh8 ablation. Blood samples for assessing the serum levels of RBP4 were collected from (±)-44-treated and control chow-treated Abca4^{-/-} mice at pre-dose and after 10 weeks of treatment. Following the 10 weeks of dosing, the eyecups of treated and untreated Abca4^{-/-}/Rdh8^{-/-} mice as well as the eyecups of the reference wild type mice were collected for the quantitative A2E analysis.

### Example 46. (±)-44 Conferred Partial Preservation of Photoreceptor Cells in the Double Knock-Out Abca4^{-/-}/Rdh8^{-/-} Mouse Model

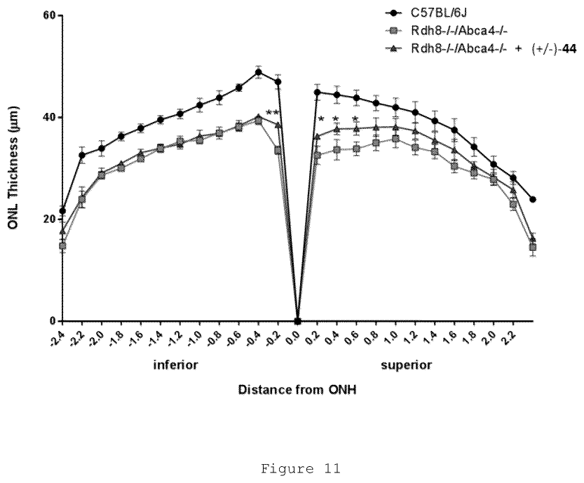

(±)-44 was formulated into a Picolab 5053 chow to ensure a daily dosing of 25 mg/kg of (±)-44. Long-term 10-week dosing of the compound formulated into a chow was conducted in Abca4^{-/-}/Rdh8-mice. The age-matched control group of wild-type C57BL/6J mice was kept on a standard Picolab 5053 chow. After 10 weeks of dosing, whole eyes were collected and fixed in the 2% glutaraldehyde/4% paraformaldehyde. Eyes were embedded in paraffin and sectioned at a thickness of 8 μm. Sections were counterstained using hematoxylin and eosin (H&E). Morphologic observations and light microscopy was performed. Outer nuclear layer (ONL) thickness was measured at 200-μm intervals superior and inferior to the edge of the optic nerve head along the vertical meridian using a digital imaging system. The ONL area was calculated as a sum of the ONL thicknesses in superior and inferior retina and multiplied by the measurement interval. Photoreceptor protection is evident at multiple points in the superior retina (FIG. 11).

### Discussion

We here describe a novel class of non-retinoid bispecific compounds capable of exhibiting dual RBP4 antagonist and TTR tetramer kinetic stabilization activity. Compounds were evaluated in three assays designed to measure (1) compound binding affinity to non-TTR associated RBP4 (scintillation proximity assay, SPA), (2) compound binding affinity to unliganded TTR tetramers (fluorescence polarization assay, FP), and (3) compound functional antagonist potency for disruption of the holo-RBP4-TTR complex (homogenous time-resolved fluorescence assay, HTRF). The results are shown in Table 1. Compound (±)-44 was less potent in RBP4 SPA binding affinity and RBP4-TTR HTRF functional antagonist activity relative to benchmark 8 ((±)-44 RBP4 SPA IC₅₀=80.0 nM; RBP4-TTR HTRF IC₅₀=0.25 μM), however, (±)-44 did present an attractive balance of dual activity for both targets ((±)-44 TTR FP IC₅₀=2.85 μM). An approximate 2-fold enantiopreference in RBP4 SPA binding affinity was observed for the R-enantiomer of (±)-44 ((R)-50 RBP4 SPA IC₅₀=65.0 nM; (S)-56 RBP4 SPA IC₅₀=150.0 nM), however there was no delineation between the enantiomers with regard to RBP4-TTR HTRF or TTR FP activity.

Compound (±)-44 exhibited excellent kinetic solubility in phosphate buffered saline (PBS) (pH 7.4) and the observed microsomal stability and CLᵢₙₜ values suggest very low predicted hepatic clearance (Table 2). The % plasma protein binding (PPB) data indicates low fraction unbound (Table 2). In addition, (±)-44 lacked limiting inhibitory activity in a standard CYP panel (Table 2). Importantly, unlike previously reported analogue 8, which exhibited ancillary PPARγ agonist activity, (±)-44 was found to be devoid of PPARγ agonist activity (Table 2).

Compound (±)-44 showed very low plasma clearance (0.0499 L/hr/kg) and a half-life of 9.9 h following administration of a single dose (2 mg/kg IV and 5 mg/kg PO) to CD-1 male mice (Table 3). The compound was well absorbed and slowly eliminated from plasma after oral administration with an observed Cₘₐₓ of 3033 ng/ml and corresponding Tₘₐₓ at 0.83 h (Table 3). Very high exposures were observed (AUC_{INF} was 52439 hr-ng/mL) and the estimated % F was 52%.

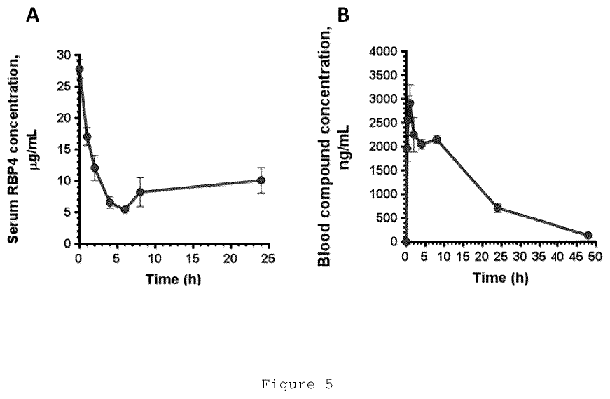

After a single 25 mg/kg oral dose of (±)-44, a maximum of the 81% reduction in serum RBP4 was observed 6 h after the dose administration (FIG. 5, A). There was no effect of compound dosing on serum TTR levels (data not shown). The dynamics of the in vivo serum RBP4 reduction demonstrated a general correlation between the presence of (±)-44 in circulation after oral dosing (FIG. 5, B) and a reduction in serum RBP4 (FIG. 5, A). The magnitude of the maximal RBP4 reduction (81%) and the duration of the RBP4 lowering effect (64% reduction at the 24 h time point) correlate well with pharmacokinetic properties of the compound such as high Cₘₐₓ, long exposure and slow clearance (Table 3).

A very good correlation has been previously established between the ability of RBP4 antagonists from different classes to induce serum RBP4 lowering and preclinical efficacy in the Abca4^{-/-} mouse model of enhanced retinal lipofuscinogenesis (Radu, R. A. et al. 2005; Dobri, N. et al. 2013; Racz, B. et al. 2018). Based on its very good RBP4 lowering activity, it seems reasonable to expect (±)-44 to be efficacious in reducing the formation of cytotoxic lipofuscin retinoids in the retina.

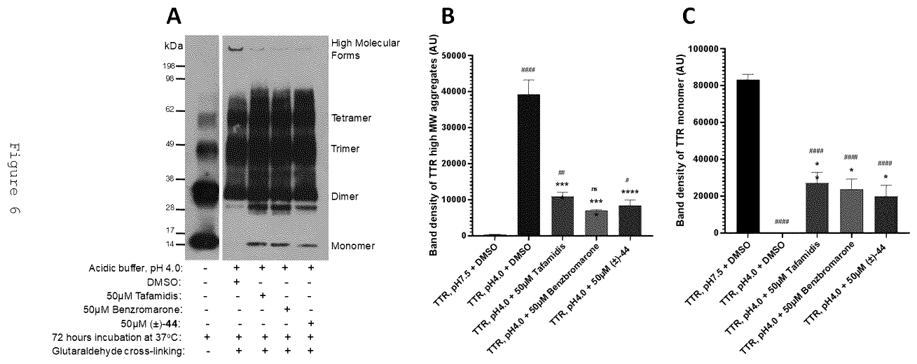

Inhibition of the acid-induced TTR aggregation in vitro is a well-established approach to characterization of TTR kinetic stabilizers (Petrassi, H. M. et al. 2005; Green, N. S. et al. 2005). Long-term 72 h incubation of TTR at 37° C. in acidic conditions leads to tetramer destabilization and dissociation followed by partial monomer denaturation and its misassembly into amyloid fibrils and other high molecular weight aggregates (Hurshman, A. R. et al. 2004). Compound (±)-44 was evaluated as a kinetic TTR stabilizer by its ability to prevent acid-mediated TTR aggregate formation using modifications of the previously published protocol and using tafamidis and benzbromarone as positive controls (Klabunde, T. et al. 2000; Niemietz, C. et al. 2018). Tafamidis is a potent TTR kinetic stabilizer approved as a therapy for familial amyloid polyneuropathy while benzbromarone, a uricosuric drug, was found in our prior experiments to be a potent TTR ligand with IC₅₀ of 293 nM in the FP TTR binding assay which is on par with the reported potency of tafamidis in this assay (Penchala, S. C. et al. 2013). Following 72 h of incubation with DMSO at pH 4.0 the high molecular forms of TTR were significantly increased while no such forms were observed after a similar incubation period at neutral pH (FIG. 6, A). Similar to the activity of two potent TTR ligands, tafamidis and benzbromarone (structure not shown), (±)-44 significantly reduced the formation of high molecular weight TTR species (FIG. 6, A) indicating that it can act as a TTR kinetic stabilizer. Higher intensities of the TTR monomer bands in samples treated with tafamidis, benzbromarone, and (±)-44 in comparison to DMSO reflected a corresponding decrease in TTR aggregation conferred by tafamidis, benzbromarone, and (±)-44. Quantitative analysis of band intensity revealed a 3.6-, 5.6-, and 4.7-fold reduction in formation of high molecular weight aggregates induced by tafamidis, benzbromarone and (±)-44, respectively (FIG. 6, B). Significant increase in the TTR monomer band intensities associated with the decrease in aggregate formations were evident in samples treated with tafamidis, benzbromarone, and (±)-44 (FIG. 6, C). Overall, the results of the aggregation experiments established that bispecific analogue (±)-44 can act as a TTR kinetic stabilizer.

Abca4^{-/-} mouse model serves as an established model for assessing preclinical efficacy of compounds inhibiting the rate of bisretinoid formation (Petrukhin, K. 2013). Long-term (12-week) daily dosing of 25 mg/kg of (±)-44 in Abca4^{-/-} mice resulted in 79% serum RBP4 reduction at the 12-week time point compared to the baseline wild type mice and 82% serum RBP4 reduction when compared to untreated Abca4^{-/-} mice (FIG. 7). Following the 12 weeks of dosing, the eyecups of treated and untreated Abca4^{-/-} mice as well as the eyecups of the reference wild type mice were collected for the quantitative A2E analysis. This analysis revealed approximately 50% A2E statistically significant reduction (p<0.0001) in the (±)-44-treated Abca4^{-/-} mice as compared to the control chow-treated Abca4^{-/-} mice (FIG. 8).

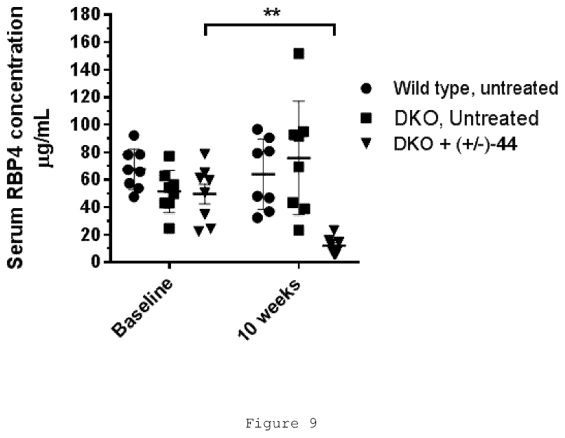

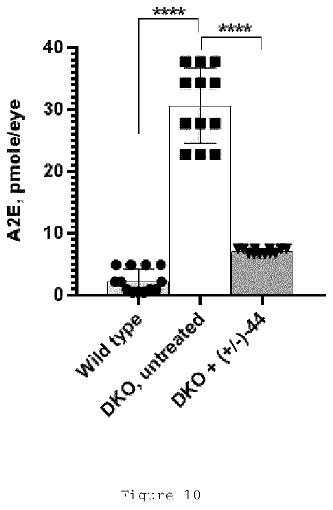

Mice lacking both the Abca4 transporter and retinol dehydrogenase 8 (Rdh8) combine the insult induced by enhanced accumulation of bisretinoids with increased exposure to retinaldehyde (Maeda, A. et al. 2008). Long-term (10-week) daily dosing of 25 mg/kg of (±)-44 in Abca4^{-/-}/Rdh8^{-/-} mice resulted in 76% serum RBP4 reduction at the 10-week time point compared to the baseline (FIG. 9). The reduction of serum RBP4 at 10 weeks was 84% in the (±)-44-treated Abca4^{-/-}/Rdh8^{-/-} mice compared to untreated Abca4^{-/-}/Rdh8^{-/-} mice. Following the 10 weeks of dosing, analysis of the eyecups of treated and untreated Abca4^{-/-}/Rdh8^{-/-} mice as well as the eyecups of the reference wild type mice revealed a 77% A2E statistically significant reduction (p<0.0001) in the (±)-44-treated Abca4^{-/-}/Rdh8^{-/-} mice as compared to the control chow-treated Abca4^{-/-}/Rdh8^{-/-} animals (FIG. 10).

The Abca4^{-/-} mouse model does not mimic certain significant aspects of Stargardt disease and dry AMD such as photoreceptor degeneration. In contrast, mice lacking both the Abca4 transporter and enzyme retinol dehydrogenase 8 (Rdh8) develop severe photoreceptor degeneration, in addition to enhanced accumulation of lipofuscin bisretinoids. (±)-44 was characterized for the ability to reduce photoreceptor degeneration in the Abca4^{-/-}/Rdh8^{-/-} mouse model. Long-term (10-week) daily dosing of 25 mg/kg of (±)-44 in Abca4^{-/-}/Rdh8^{-/-} mice resulted in photoreceptor protection at multiple points in the superior retina (FIG. 11).

The compounds described herein are shown to possess properties useful for the treatment of indications described above.

### REFERENCES

Adams, D. et al. Expert Opin. Pharmacother. 17, 791-802 (2016).
Adams, W. R. et al. Proc. Soc. Exp. Biol. Med. 208, 178-185 (1995).
Aeberli, I. et al. J. Clin. Endocrinol. Metab. 92, 4359-4365 (2007)
Alexander, K. M. & Falk, R. H. Circ.: Heart Failure 9, e003489 (2016)
Alhamadsheh, M. M. et al. Sci. Transl. Med. 3, 97ra81 (2011).
Almeida, M. R. et al. Biochem. J. 381, 351-356 (2004).
Almeida, M. R. et al. Curr. Drug Targets CNS Neurol. Disord. 4, 587-596 (2005).
Ben-Shabat, S. et al. J. Biol. Chem. 277, 7183-7190 (2002).
Bergmann, M. et al. FASEB J. 18, 562-564 (2004).
Berk, J. L. et al. Amyloid 19, 37-38 (2012).
Berni, R. & Formelli, F. FEBS Lett. 308, 43-45 (1992).
Bulawa, C. E. et al. Proc. Natl. Acad. Sci. U.S.A. 109, 9629-9634 (2012).
Brunjes, D. L. et al. J. Card. Failure 22, 996-1003 (2016).
Buxbaum, J. N. et al. Genet. Med., 19, 733-742 (2017).
Cioffi, C. L. et al. J. Med. Chem. 57, 7731-7757 (2014).
Cioffi, C. L. et al. J. Med. Chem. 58, 5863-5888 (2015).
Cioffi, C. L. et al. J. Med. Chem. 62, 5470-5500 (2019).
Clagett-Dame, M. & DeLuca, H. F. Annu. Rev. Nutr. 22, 347-381 (2002).
Coelho, T. et al. Neurol. Ther. 5, 1-25 (2016).
Connors, L. H. et al. Amyloid 18, 157-159 (2011).
De, S. & Sakmar, T. P. J. Gen. Physiol. 120, 147-157 (2002).
Delori, F. C. In Retinal Pigment Epithelium and Macular Disease (Documenta Ophthalmologica), G. Coscas, F. C. P., Ed. Kluwer Academic Publishers: Dordrecht, The Netherlands 1995; Vol. 62, pp 37-45.
Dobri, N. et al. Invest. Ophth. Vis. Sci. 54, 85-95 (2013).
Dontsov, A. E. et al. Dokl. Biochem. Biophys. 425, 98-101 (2009).
Dorey, C. K. et al. Invest. Ophth. Vis. Sci. 30, 1691-1699 (1989).
Falk, R. H. et al. N. Engl. J. Med. 337, 898-909 (1997).
Finnemann, S. C. et al. Proc. Natl. Acad. Sci. U.S.A. 99, 3842-3847 (2002).
Foss, T. R. et al. Biochemistry 44, 15525-15533 (2005).
Graham, T. E. et al. N. Engl. J. Med. 354, 2552-2563 (2006).
Green, N. S. et al. Proc. Nat. Acad. Sci. U.S.A. 102, 14545-14550 (2005).
Holz, F. G. et al. Graefes Arch. Clin. Exp. Ophthalmol. 237, 145-152 (1999).
Holz, F. G. et al. Invest. Ophth. Vis. Sci. 42, 1051-1056 (2001).
Holz, F. G. et al. Am. J. Ophthalmol. 143, 463-472 (2007).
Hurshman, A. R. et al. Biochemistry 43, 7365-7381 (2004).
Hyung, S. J. et al. ACS. Chem. Biol. 5, 1137-1146 (2010).
Ingelsson, E. et al. Atherosclerosis 206, 239-244 (2009).
Johnson, S. M. et al. Acc. Chem. Res. 38, 911-921 (2005).
Judge, D. P. et al. J. Am. Coll. Cardiol. 74, 285-295 (2019).
Kerschen, P. & Plante-Bordeneuve, V. Curr. Treat. Options Neurol. 18, 53 (2016).
Klabunde, T. et al. Nat. Struct. Biol. 7, 312-321 (2000).
Kiser, P. D. et al. Chem. Rev. 114, 194-232 (2014).
Kowalska, I. et al. J. Clin. Endocrinol. Metab. 93, 2786-2789 (2008).
Lamb, Y. N. & Deeks, E. D. Drugs 79, 863-874 (2019).
Lee, S. A. et al. Hepatology 64, 1534-1546 (2016).
Maeda, A. et al. J. Biol. Chem. 283, 26684-26693 (2008).
Mata, N. L. et al. Retina 33, 498-507 (2013).
Miller, M. et al. J. Med. Chem. 61, 7862-7876 (2018).
Motani, A. et al. J. Biol. Chem. 284, 7673-7680 (2009).
Nencetti, S. & Orlandini, E. Curr. Med. Chem. 19, 2356-2379 (2012).
Nencetti, S. et al. ChemMedChem 8, 1617-1619 (2013).
Niemietz, C. et al. J. Cell Sci. 131, pii: jcs219824 (2018).
Norseen, J. et al. Mol. Cell. Biol. 32, 2010-2019 (2012).
Palczewski, K. Trends Pharmacol. Sci. 31, 284-295 (2010).
Penchala, S. C. et al. Proc. Natl. Acad. Sci. U.S.A 110, 9992-9997 (2013).
Petrassi, H. M. et al. J. Am. Chem. Soc. 127, 6662-6671 (2005).
Petrukhin, K., Expert Opin. Ther. Tar. 11, 625-639 (2007).
Petrukhin, K., Drug Discov. Today Ther. Strateg. 10, e11-e20 (2013).
Qi, Q. et al. J. Clin. Endocrinol. Metab. 92, 4827-4834 (2007).
Racz, B. et al. J. Biol. Chem. 293, 11574-11588 (2018).
Racz, B. et al. PLoS One 15, e0228291 (2020).
Radu, R. A. et al. Invest. Ophth. Vis. Sci., 46, 4393-4401 (2005).
Radu, R. A. et al. J. Biol. Chem. 286, 18593-18601 (2011).
Rodrigues, D. A. Drug-Drug Interactions, 2nd ed.; CRC Press: New York, 2008.
Rozanowska, M. et al. J. Biol. Chem. 270, 18825-18830 (1995).
Ruberg, F. L. & Berk, J. L. Circulation 126, 1286-1300 (2012).
Sant'Anna, R. et al. Nat. Commun. 7, 10787 (2016).
Schmitz-Valckenberg, S. et al. Surv. Ophthalmol. 54, 96-117 (2009).
Sekijima, Y. et al. Lab. Investig. 83, 409-417 (2003).
Sparrow, J. R. & Cai, B. Invest. Ophth. Vis. Sci. 42, 1356-1362 (2001).
Sparrow, J. R. et al. Invest. Ophth. Vis. Sci. 40, 2988-2995 (1999).
Sparrow, J. R. et al. Invest. Ophth. Vis. Sci. 43, 1222-1227 (2002).
Sparrow, J. R. et al. Vis. Res. 43, 2983-2990 (2003).
Sparrow, J. R. et al. Prog. Retin. Eye Res. 31, 121-135 (2012).
Steinmetz, A. C. et al. Annu. Rev. Biophys. Biomol. Struct. 30, 329-359 (2001).
Suter, M. et al. J. Biol. Chem. 275, 39625-39630 (2000).
Ton, V. K. et al. Clin. Med. Insights: Cardiol. 8s1, 39-44 (2014).
Tsin, A. et al. J. Biol. Chem. 293, 13016-13021 (2018).
Vives-Bauza, C. et al. J. Biol. Chem. 283, 24770-24780 (2008).
Weng, J. et al. Cell 98, 13-23 (1999).
Westermark, P. et al. Amyloid. 10, 48-54 (2003).
White, J. T. & Kelly, J. W. Proc. Natl. Acad. Sci. U.S.A. 98, 13019-13024 (2001).
Wolf, G. Physiological Rev. 64, 873-937 (1984).
Yang, Q. et al. Nature 436, 356-362 (2005).
Young, R. W. Surv Ophthalmol. 31, 291-306 (1987).
Zhou, J. et al. Proc. Natl. Acad. Sci. U.S.A. 103, 16182-16187 (2006).

1. A compound having the structure: 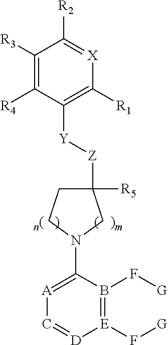
wherein
X is CR₆ or N;
R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), -O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇,
—SO₂R₇, —NHSO₂R₇, —OC(O)R₇, —SC(O)R₇, —NHC(O)R₈ or —NHC(S) Re, wherein R₇ is, H, -(alkyl), —OH, —O(alkyl), —NH₂, —NH(alkyl) or —N(alkyl)₂, and
wherein R₈ is, -(alkyl), —O-(alkyl), —NH₂, —NH(alkyl) or —N(alkyl)₂;
Y is O, S, N, NH, or a bond;
Z is O, S, N, NH, (CH₂)ₒ, or a bond;
R₅ is H, OH, halogen, alkyl, or R₅ is (CH₂) p and is bound to Y when Y is N to form a ring together with Z;
and p are independently 0, 1, 2, or 3;
m and n are independently 0, 1, 2, 3, or 4;
A, C, and D are each independently N or CR₉;
R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, -CO(O-alkyl);
B and E are N, CR₉, or CFG wherein at least one of B or E is CFG;
F is absent or present, and when present is 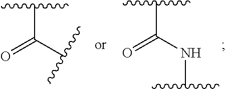
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, S02 (alkyl), SO₂(cycloalkyl), SO₂(cycloalkylalkyl), CH₂NHR₁₀, CH₂NR₁₀R₁₁, or CH₂COOR₁₀, wherein each R₁₀ and R₁₁ are each independently H, alkyl, cycloalkyl, —C(O)-alkyl, —C(O)-cycloalkyl, —C(O)OH, —C(O)-O-alkyl, —C(O)—O-cycloalkyl, —C(O)NH₂, —C(O)NH(alkyl), -C(O)NH(cycloalkyl), —C(O)N(alkyl)₂, —CH₂NH(alkyl), —CH₂COOH, —SO₂CH₃, —OH, -O (alkyl), —NH₂, —NH(alkyl), —N(alkyl)₂,
or a pharmaceutically acceptable salt thereof.

2. The compound of claim 1, wherein m is 1 or 2 and n is 0, 1, or 2; or
wherein m is 1 and n is 1.

3. (canceled)

4. The compound of claim 2, wherein Y and Z are each independently CH₂, O, S, or NH; or
wherein Y is O and Z is CH₂.

5. (canceled)

6. The compound of claim 4, wherein A and B are N, C and D are CR₉, and E is CFG; or
wherein A, B, C and D are CR₉, and E is CFG; or
wherein A is N, B is CFG, C, D and E are each CR₉; or
wherein A is N, B, C, and D are each CR₉ and E is CFG; or
wherein A, C, and D are each CR₉, B is N, and E is CFG;
or
wherein
X is CR₆ or N;
R₁, R₂, R₃, R₄, and R₆ are each independently H, t-butyl, cyclopentyl, cyclohexyl, CF₃, F, Cl, CN, or —OCH₃;
or
wherein
R₁ or R₄ is CF₃;
or
wherein
X is CR₆;
R₁ is CF₃ and R₂, R₃, R₄ and R₆ are each independently H, t-butyl, cyclopentyl, cyclohexyl, CF₃, F, Cl, CN, or —OCH₃.

7-9. (canceled)

10. The compound of claim 2, having the structure: 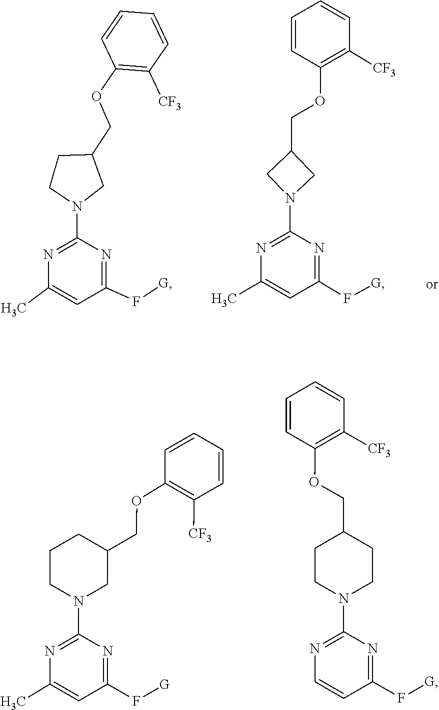
wherein
C is CR₉;
R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, —CO(O-alkyl);
F is absent or present, and when present is 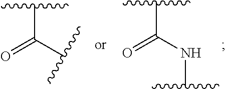
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein each R₁₀ and R₁₁ are each independently H, alkyl, cycloalkyl, —C(O)-alkyl, —C(O)-cycloalkyl, —C(O)OH, —C(O)—O— alkyl, —C(O)-O-cycloalkyl, —C(O) NH₂, —C(O) NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl), —N(alkyl)₂,
or a pharmaceutically acceptable salt thereof.

11. The compound of claim 10, wherein C is CR₉ and R₉ is H, -alkyl, —O(alkyl) or —NH(alkyl); or
wherein R₉ is -alkyl.

12. (canceled)

13. The compound of any one of claim 10 having the structure: 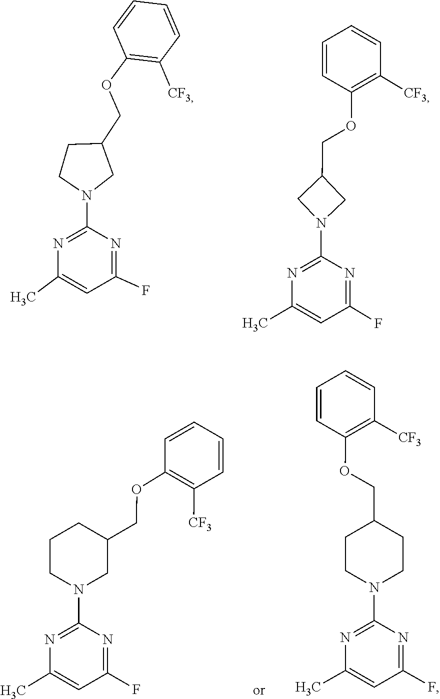
wherein F is substituted or unsubstituted heteroaryl group, or
wherein F has the structure: 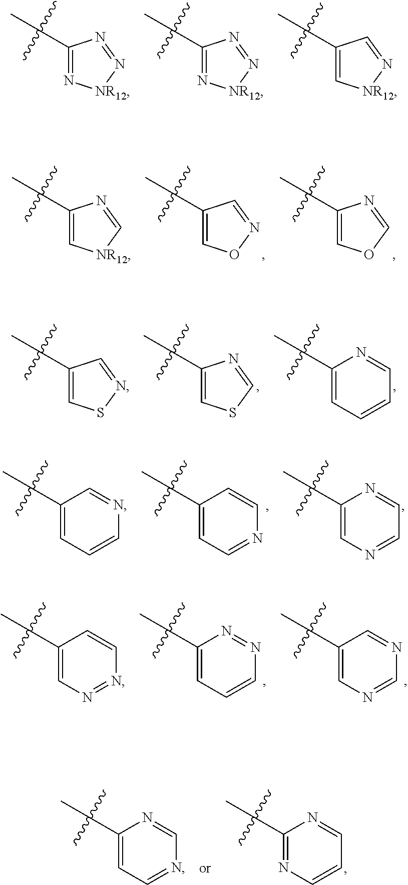
and R₁₂ is H, -(alkyl), -(alkenyl) or -(alkynyl).

14. (canceled)

15. The compound of claim 1 having the structure: 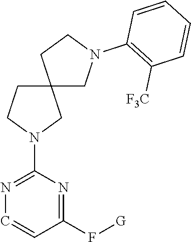
wherein
C is CR₉;
R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, —CO(O-alkyl);
F is absent or present, and when present is 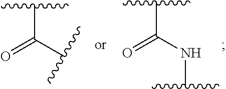
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein each R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O) NH₂, —C(O)NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl), —N(alkyl)₂,
or a pharmaceutically acceptable salt thereof.

16. The compound of claim 15, wherein C is CR9 and R9 is H, -alkyl, —O(alkyl) or —NH(alkyl); or
wherein R9 is -alkyl.

17. The compound of claim 1 having the structure: 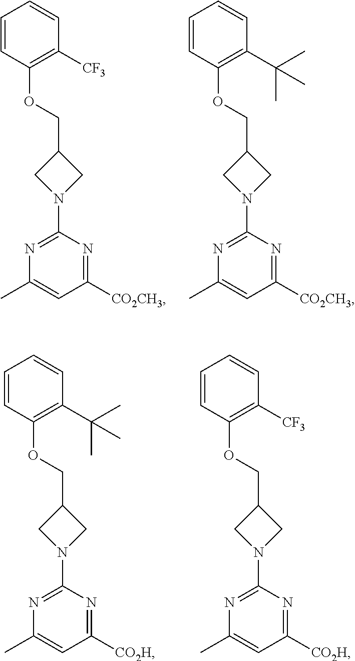 [SMILES: COC(=O)c1cc(C)nc(N2CC(COc3ccccc3C(F)(F)F)C2)n1 ; COC(=O)c1cc(C)nc(N2CC(COc3ccccc3C(C)(C)C)C2)n1 ; Cc1cc(C(=O)O)nc(N2CC(COc3ccccc3C(C)(C)C)C2)n1 ; Cc1cc(C(=O)O)nc(N2CC(COc3ccccc3C(F)(F)F)C2)n1] 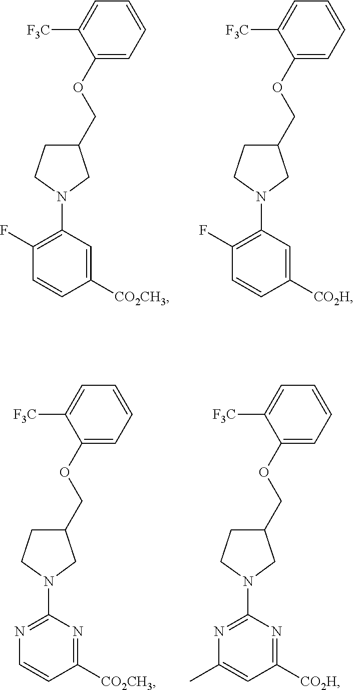 [SMILES: COC(=O)c1ccc(F)c(N2CCC(COc3ccccc3C(F)(F)F)C2)c1 ; O=C(O)c1ccc(F)c(N2CCC(COc3ccccc3C(F)(F)F)C2)c1 ; COC(=O)c1ccnc(N2CCC(COc3ccccc3C(F)(F)F)C2)n1 ; Cc1cc(C(=O)O)nc(N2CCC(COc3ccccc3C(F)(F)F)C2)n1] 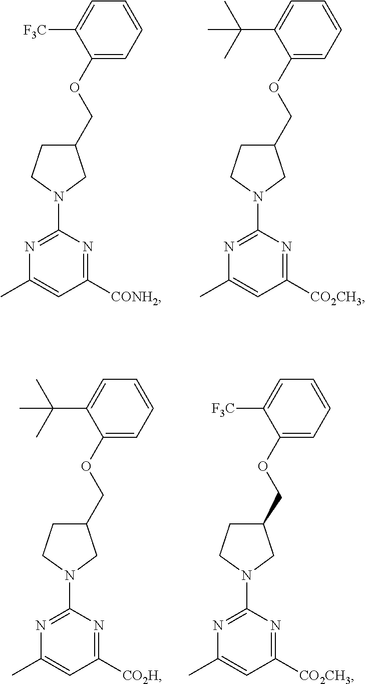 [SMILES: Cc1cc(C(N)=O)nc(N2CCC(COc3ccccc3C(F)(F)F)C2)n1 ; COC(=O)c1cc(C)nc(N2CCC(COc3ccccc3C(C)(C)C)C2)n1 ; Cc1cc(C(=O)O)nc(N2CCC(COc3ccccc3C(C)(C)C)C2)n1 ; COC(=O)c1cc(C)nc(N2CC[C@@H](COc3ccccc3C(F)(F)F)C2)n1] 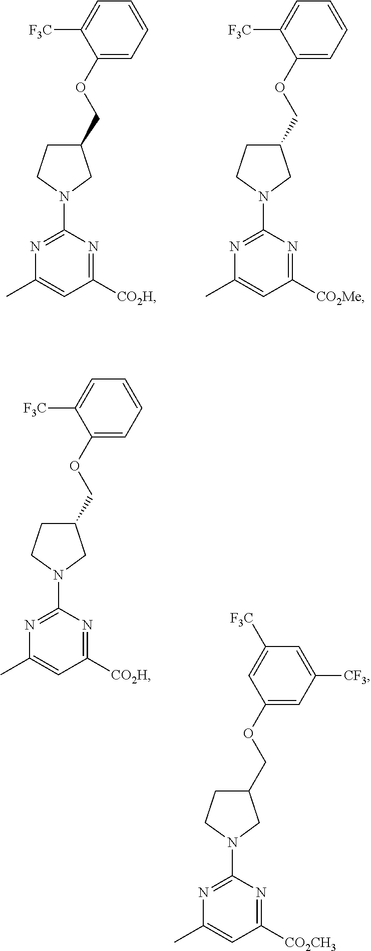 [SMILES: Cc1cc(C(=O)O)nc(N2CC[C@@H](COc3ccccc3C(F)(F)F)C2)n1 ; COC(=O)c1cc(C)nc(N2CC[C@H](COc3ccccc3C(F)(F)F)C2)n1 ; Cc1cc(C(=O)O)nc(N2CC[C@H](COc3ccccc3C(F)(F)F)C2)n1 ; COC(=O)c1cc(C)nc(N2CCC(COc3cc(C(F)(F)F)cc(C(F)(F)F)c3)C2)n1] 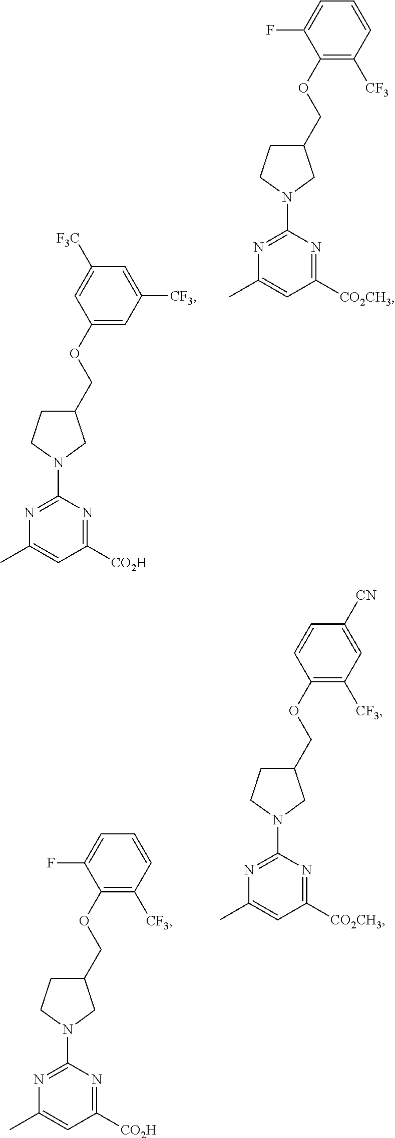 [SMILES: Cc1cc(C(=O)O)nc(N2CCC(COc3cc(C(F)(F)F)cc(C(F)(F)F)c3)C2)n1 ; COC(=O)c1cc(C)nc(N2CCC(COc3c(F)cccc3C(F)(F)F)C2)n1 ; Cc1cc(C(=O)O)nc(N2CCC(COc3c(F)cccc3C(F)(F)F)C2)n1 ; COC(=O)c1cc(C)nc(N2CCC(COc3ccc(C#N)cc3C(F)(F)F)C2)n1] 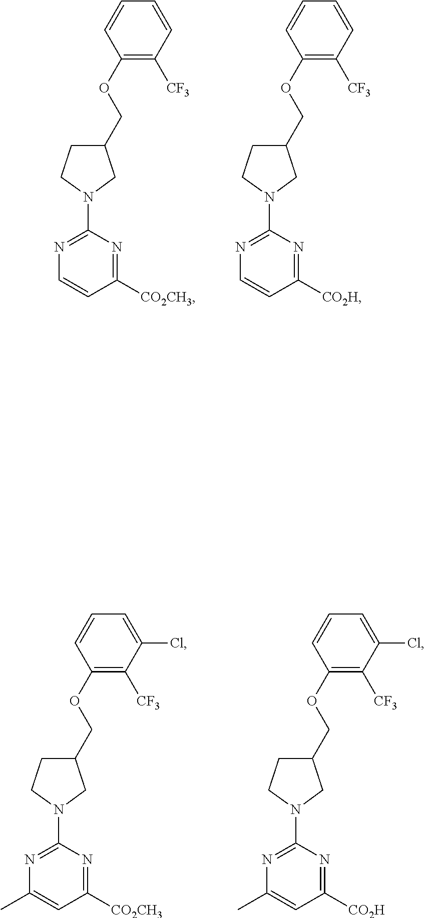 [SMILES: COC(=O)c1ccnc(N2CCC(COc3ccccc3C(F)(F)F)C2)n1 ; O=C(O)c1ccnc(N2CCC(COc3ccccc3C(F)(F)F)C2)n1 ; COC(=O)c1cc(C)nc(N2CCC(COc3cccc(Cl)c3C(F)(F)F)C2)n1 ; Cc1cc(C(=O)O)nc(N2CCC(COc3cccc(Cl)c3C(F)(F)F)C2)n1] 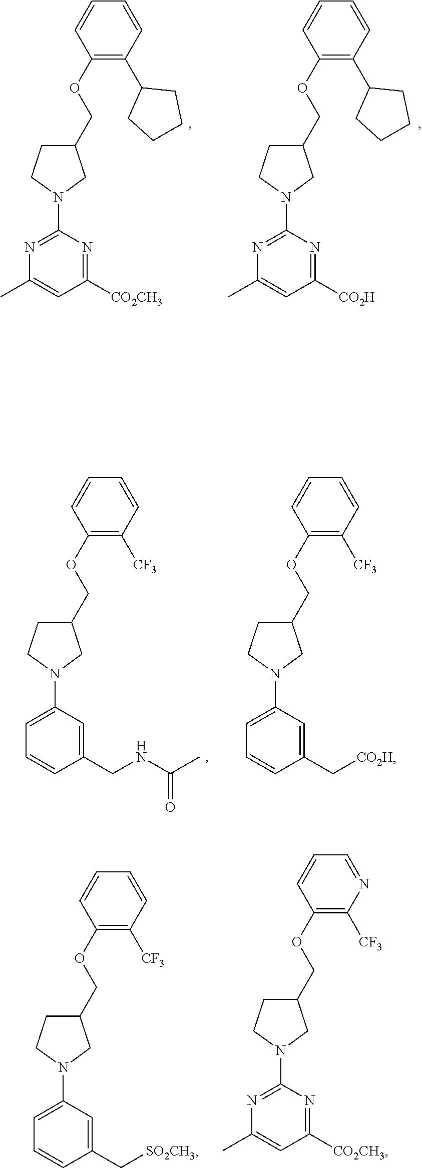 [SMILES: COC(=O)c1cc(C)nc(N2CCC(COc3ccccc3C3CCCC3)C2)n1 ; Cc1cc(C(=O)O)nc(N2CCC(COc3ccccc3C3CCCC3)C2)n1 ; CC(=O)NCc1cccc(N2CCC(COc3ccccc3C(F)(F)F)C2)c1 ; O=C(O)Cc1cccc(N2CCC(COc3ccccc3C(F)(F)F)C2)c1 ; CS(=O)(=O)Cc1cccc(N2CCC(COc3ccccc3C(F)(F)F)C2)c1 ; COC(=O)c1cc(C)nc(N2CCC(COc3cccnc3C(F)(F)F)C2)n1] 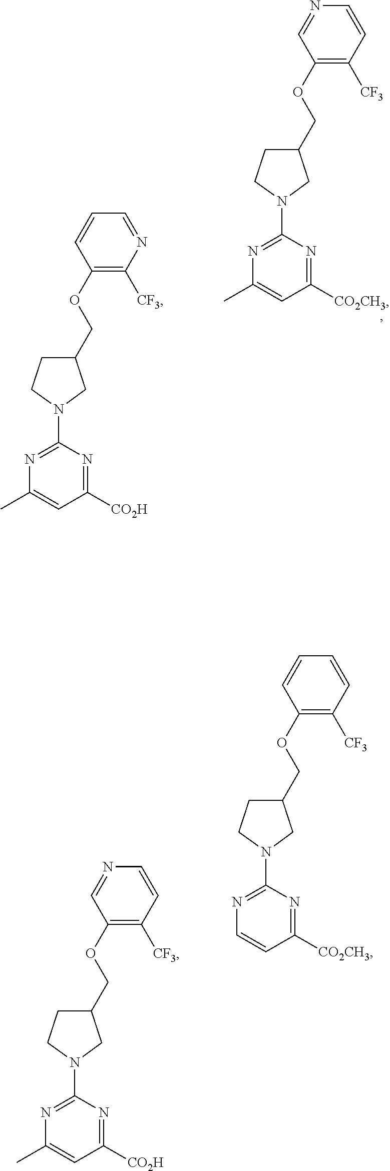 [SMILES: Cc1cc(C(=O)O)nc(N2CCC(COc3cccnc3C(F)(F)F)C2)n1 ; COC(=O)c1cc(C)nc(N2CCC(COc3cnccc3C(F)(F)F)C2)n1 ; Cc1cc(C(=O)O)nc(N2CCC(COc3cnccc3C(F)(F)F)C2)n1 ; COC(=O)c1ccnc(N2CCC(COc3ccccc3C(F)(F)F)C2)n1] 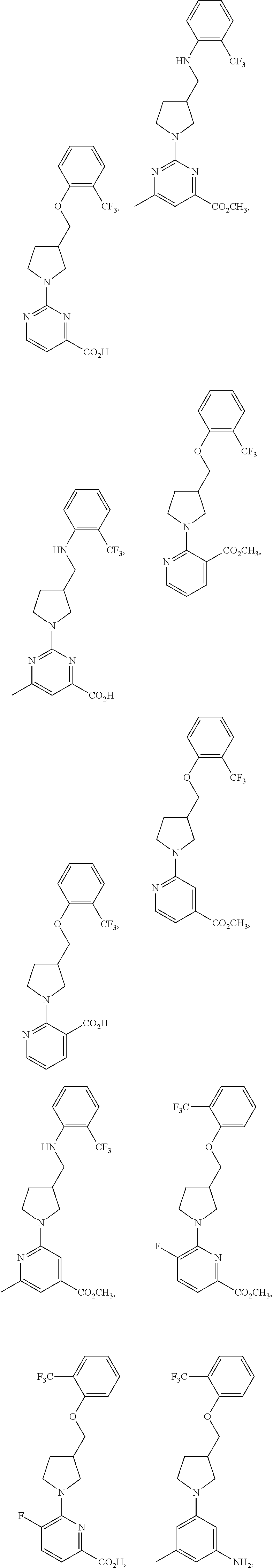 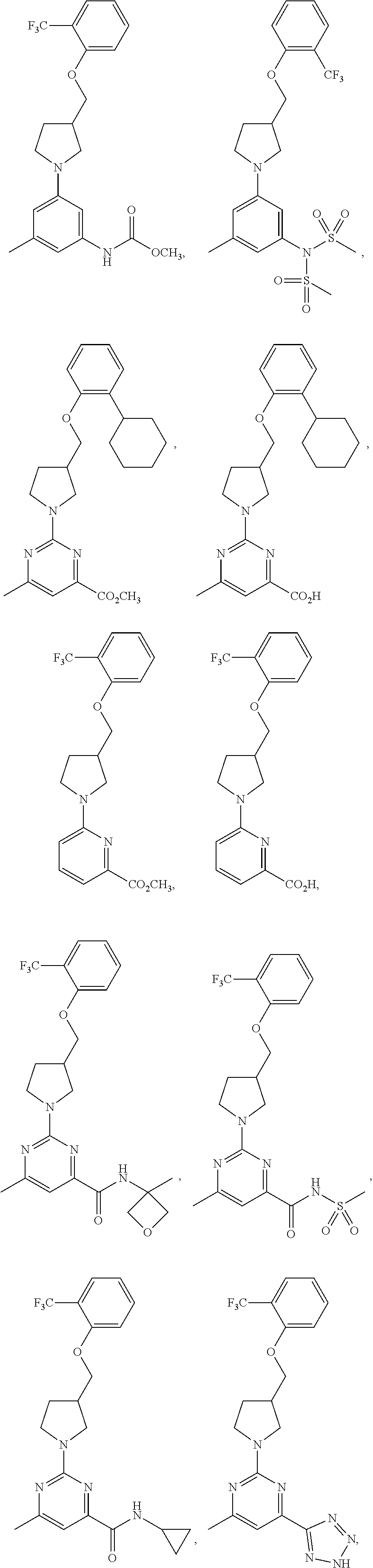 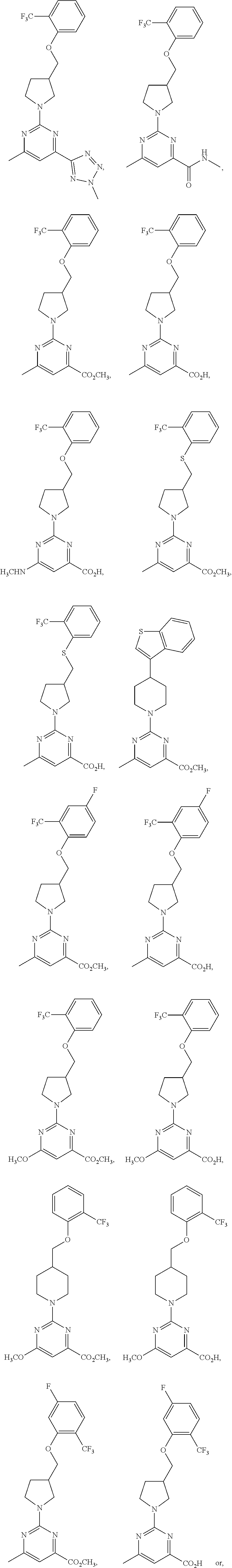
or a pharmaceutically acceptable salt of the compound.

18. The compound of claim 1 having the structure: 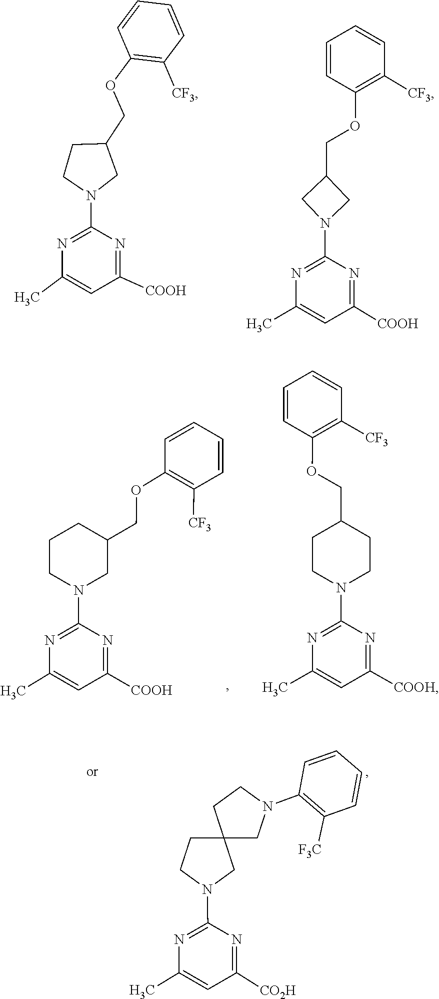 [SMILES: Cc1cc(C(=O)O)nc(N2CCC(COc3ccccc3C(F)(F)F)C2)n1 ; Cc1cc(C(=O)O)nc(N2CC(COc3ccccc3C(F)(F)F)C2)n1 ; Cc1cc(C(=O)O)nc(N2CCCC(COc3ccccc3C(F)(F)F)C2)n1 ; Cc1cc(C(=O)O)nc(N2CCC(COc3ccccc3C(F)(F)F)CC2)n1 ; Cc1cc(C(=O)O)nc(N2CCC3(CCN(c4ccccc4C(F)(F)F)C3)C2)n1]
or a pharmaceutically acceptable salt thereof.

19-22. (canceled)

23. A pharmaceutical composition comprising the compound of claim 1 and a pharmaceutically acceptable carrier.

24. A method for stabilizing TTR tetramers in a mammal comprising administering to the mammal an amount of a composition of claim 23 effective to stabilizing TTR tetramers.

25. A method for treating a disease characterized by excessive lipofuscin accumulation in the retina, or a TTR amyloidosis (ATTR) disease, or both a disease characterized by excessive lipofuscin and a TTR amyloidosis (ATTR) disease, in a mammal afflicted therewith comprising administering to the mammal an effective amount of a composition of claim 23.

26. The method of claim 25, wherein the disease is further characterized by bisretinoid-mediated macular degeneration.

27. The method of claim 26, wherein the amount of the compound is effective to lower the serum concentration of RBP4 in the mammal, or wherein the amount of the compound is effective to lower the retinal concentration of a bisretinoid in lipofuscin in the mammal; or
the method wherein the amount of the compound is effective to stabilize TTR tetramers in the mammal.

28. (canceled)

29. The method of claim 27, wherein the bisretinoid is A2E; or
wherein the bisretinoid is isoA2E; or
wherein the bisretinoid is A2-DHP-PE; or
wherein the bisretinoid is atRAL di-PE.

30-32. (canceled)

33. The method of claim 25, wherein the disease characterized by excessive lipofuscin accumulation in the retina is Age-Related Macular Degeneration; or
wherein the disease characterized by excessive lipofuscin accumulation in the retina is dry (atrophic) Age-Related Macular Degeneration; or
wherein the disease characterized by excessive lipofuscin accumulation in the retina is Stargardt Disease; or
wherein the disease characterized by excessive lipofuscin accumulation in the retina is Best disease; or
wherein the disease characterized by excessive lipofuscin accumulation in the retina is adult vitelliform maculopathy; or
wherein the disease characterized by excessive lipofuscin accumulation in the retina is Stargardt-like macular dystrophy.

34-38. (canceled)

39. The method of claim 1, wherein the administration is effective to reduce photoreceptor degeneration; or
wherein the method is further effective to stabilize TTR tetramers in the mammal; or
wherein the mammal is further afflicted with a TTR amyloidosis (ATTR) disease and the method is effective for treating the TTR amyloidosis (ATTR) disease in the mammal.

40-41. (canceled)

42. The method of claim 39, wherein the TTR amyloidosis (ATTR) disease is senile systemic amyloidosis (SSA); or
wherein the TTR amyloidosis (ATTR) disease is peripheral polyneuropathy (ATTR-PN); or
wherein the TTR amyloidosis (ATTR) disease is cardiomyopathy (ATTR-CM); or
wherein the TTR amyloidosis (ATTR) disease is characterized by deposition of amyloid aggregates.

43-45. (canceled)
